(19) United States
(12) Patent Application Publication
(10) Pub. No.: US 2018/0057580 A1
(43) Pub. Date: Mar. 1, 2018

(54) ANTI-GREMLIN-1 (GREM1) ANTIBODIES AND METHODS OF USE THEREOF FOR TREATING PULMONARY ARTERIAL HYPERTENSION

(71) Applicant: Regeneron Pharmaceuticals, Inc., Tarrytown, NY (US)

(72) Inventors: Dan Chalothorn, New York, NY (US); Lori C. Morton, Chappaqua, NY (US)

(21) Appl. No.: 15/684,073

(22) Filed: Aug. 23, 2017

Related U.S. Application Data
(60) Provisional application No. 62/380,562, filed on Aug. 29, 2016.

(51) Int. Cl. C07K 16/24; A61K 39/395; A61K 45/06

(52) U.S. Cl. CPC C07K 16/24; A61K 2039/505; A61K 45/06; A61K 39/3955

(57) ABSTRACT

The present invention provides anti-Gremlin-1 (GREM1) antibodies, and antigen-binding fragments thereof, as well as methods of use of such antibodies, or antigen-binding fragments thereof, for treating a subject having pulmonary arterial hypertension (PAH).

### RELATED APPLICATIONS

This application claims the benefit of priority to U.S. Provisional Patent Application No.: 62/380,562, filed on Aug. 29, 2016, the entire contents of which are hereby incorporated herein by reference.

### SEQUENCE LISTING

The instant application contains a Sequence Listing which has been submitted electronically in ASCII format and is hereby incorporated by reference in its entirety. Said ASCII copy, created on Aug. 17, 2017, is named 118003_28802_SL.txt and is 195,927 bytes in size.

### BACKGROUND OF THE INVENTION

Pulmonary arterial hypertension (PAH) is a progressive disorder characterized by a sustained increase in pulmonary artery pressure that damages both the large and small pulmonary arteries. PAH is defined hemodynamically as a systolic pulmonary artery pressure greater than 30 mm Hg or evaluation of mean pulmonary artery pressure greater than 25 mm Hg with a pulmonary capillary or left atrial pressure equal to or less than 15 mm Hg. See, e.g., Zaiman et al., Am. J. Respir. Cell Mol. Biol. 33:425-31 (2005). The persistent vasoconstriction in PAH leads to structural remodeling during which pulmonary vascular smooth muscle cells and endothelial cells undergo a phenotypic switch from a contractile normal phenotype to a synthetic phenotype leading to cell growth and matrix deposition. As the walls of the smallest blood vessels thicken, they are less able to transfer oxygen and carbon dioxide normally between the blood and the lungs and, in time, pulmonary hypertension leads to thickening of the pulmonary arteries and narrowing of the passageways through which blood flows. Eventually, the proliferation of vascular smooth muscle and endothelial cells leads to remodeling of the vessels with obliteration of the lumen of the pulmonary vasculature. Histological examination of tissue samples from patients with pulmonary hypertension shows intimal thickening, as well as smooth muscle cell hypertrophy, especially for those vessels <100 μm diameter. This causes a progressive rise in pulmonary pressures as blood is pumped through decreased lumen area. As a consequence, the right side of the heart works harder to compensate and the increased effort causes the right ventricle to become enlarged and thickened. The enlarged right ventricle places a person at risk for pulmonary embolism because blood tends to pool in the ventricle and in the legs. If clots form in the pooled blood, they may eventually travel and lodge in the lungs. Eventually, the additional workload placed on the right ventricle causes the heart to fail and leads to premature death in these patients.

Standard therapies for treatment of subjects having PAH are primarily hemodynamic, influencing vessel tone and include, e.g., prostacyclin analogs, endothelin receptor antagonists, phosphodiesterase inhibitors and soluble guanylate cyclases activators/stimulators, which provide symptomatic relief and improve prognosis. However, these therapies fall short and do not re-establish the structural and functional integrity of the lung vasculature to provide a patient having PAH with handicap-free long-term survival.

There are many cellular pathways that could lead to the development of PAH and the structural remodeling in PAH such as, for example, the transforming growth factor-beta (TGF-β) pathway and/or bone morphogenic protein (BMP) pathway. A pathogenic role for members of the TGF-β superfamily in PAH has been suggested by the discovery that mutations in genes encoding the TGF-β receptor superfamily proteins BMPR2, ACVRL1, or ENG, or the signal transducer, SMAD9, which increase a person's susceptibility to heritable forms of PAH. It has also been shown that PAH patients have reduced BMPR2 expression/signaling (Atkinson et al. Circulation. 105(14):1672-1678, 2002; Alastalo et al. J. Clin. Invest. 121:3735-3746, 2011), that TGF-β activation of pulmonary artery smooth muscle cells is insensitive to growth inhibition with loss of BMPR2 (Morrell et al. Circulation. 104(7):790-7952001; Yang et al. Circ. Res. 102, 1212-1221, 2008), and that BMP9 activation of BMPR2 reverses preclinical PAH (Long et al. Nat Med. 21: 777-785, 2015). Biological responses to BMPs are negatively regulated by BMP antagonists that can directly associate with BMPs and inhibit receptor binding.

One such antagonist of that can directly associate with BMPs and inhibit receptor binding and BMP signaling is human gremlin-1 (GREM1), a member of the cysteine knot superfamily, (Hsu, D. R., et al 1998, Mol. Cell 1: 673-683) that binds with high affinity to BMP2, BMP4 and BMP7 (Yanagita, et al. (2005) Cytokine Growth Factor Rev 16:309-317). GREM1 has been found to be elevated in the wall of small intrapulmonary vessels of mice during hypoxia. Haploinsufficiencey of gremlin 1 augments BMP signaling and has been associated with reduce vascular resistance by inhibiting vascular remodeling (Cahill, et al. (2012) Circulation 125(7):920-30). In addition, GREM1 expression increases in human pulmonary endothelial cells under hypoxia (Costello, et al. (2008) Am J Physiol Lung Cell Mol Physiol 295(2):L272-84) and GREM1 is expressed in remodeled vessels in lungs of idiopathic and hereditary PAH patients (Cahill, et al. (2012) Circulation 125(7):920-30).

However, despite all the advances in the therapy of PAH there is as yet no prospect of cure of this deadly disease and the majority of patients continue to progress to right ventricular failure. Thus, there is a need in the art for clinically beneficial methods and compositions that target vascular remodeling regulated by the TGFβ and BMP pathways to decrease TGFβ signaling and increase BMP signaling by inhibiting GREM1.

### SUMMARY OF THE INVENTION

The present invention is based, at least in part, on the discovery that anti-gremlin-1 (GREM1) antibodies, or antigen-binding fragments thereof, are effective for ameliorating the effects of vascular remodeling in animal models of pulmonary arterial hypertension.

Accordingly, in one aspect, the present invention provides methods for treating a subject having pulmonary arterial hypertension (PAH). The methods include administering to the subject a therapeutically effective amount of an anti-GREM1 antibody, or antigen-binding fragment thereof, wherein administration of the anti-GREM11 antibody, or antigen-binding fragment thereof, to the subject inhibits thickening of the pulmonary artery in the subject, thereby treating the subject having PAH.

In another aspect, the present invention provides methods of treating a subject having pulmonary arterial hypertension (PAH). The methods include administering to the subject a therapeutically effective amount of an anti-GREM1 antibody, or antigen-binding fragment thereof, wherein administration of the anti-GREM1 antibody, or antigen-binding fragment thereof, to the subject increases stroke volume in the subject, thereby treating the subject having PAH.

In yet another aspect, the present invention provides methods of treating a subject having pulmonary arterial hypertension (PAH). The methods include administering to the subject a therapeutically effective amount of an anti-GREM1 antibody, or antigen-binding fragment thereof, wherein administration of the anti-GREM1 antibody, or antigen-binding fragment thereof, to the subject increases right ventricle cardiac output in the subject, thereby treating the subject having PAH.

In another aspect, the present invention provides methods of treating a subject having pulmonary arterial hypertension (PAH). The methods include administering to the subject a therapeutically effective amount of an anti-GREM1 antibody, or antigen-binding fragment thereof, wherein administration of the anti-GREM1 antibody, or antigen-binding fragment thereof, to the subject extends survival time of the subject, thereby treating the subject having PAH.

In one embodiment, the subject is human.

In one embodiment, the subject has Group I (WHO) PAH.

The methods of the invention may further include administering to the subject at least one additional therapeutic agent, such as an anticoagulant, a diuretic, a cardiac glycoside, a calcium channel blocker, a vasodilator, a prostacyclin analogue, an endothelium antagonist, a phosphodiesterase inhibitor, an endopeptidase inhibitor, a lipid lowering agent, and/or a thromboxane inhibitor.

Antibodies, or antigen-binding fragments thereof, for use in the present invention may block GREM1 binding to one of bone morphogenetic protein-2 (BMP2), BMP4, BMP7 or heparin.

In one embodiment, the antibody, or antigen-binding fragment thereof, exhibits one or more properties selected from the group consisting of:

(a) binds GREM1 at 37° C. with a binding dissociation equilibrium constant (K_{D}) of less than about 275 nM as measured by surface plasmon resonance;

(b) binds to GREM1 at 37° C. with a dissociative half-life (t½) of greater than about 3 minutes as measured by surface plasmon resonance;

(c) binds GREM1 at 25° C. with a K_{D} of less than about 280 nM as measured by surface plasmon resonance;

(d) binds to GREM1 at 25° C. with a t½ of greater than about 2 minutes as measured by surface plasmon resonance;

(e) blocks GREM1 binding to BMP4 with an IC₅₀ of less than about 1.9 nM as measured in a competition ELISA assay at 25° C.;

(f) blocks GREM1-mediated inhibition of BMP signaling and promotes cell differentiation; and

(g) blocks GREM1 binding to heparin.

In another embodiment, the antibody, or antigen-binding fragment thereof, competes for specific binding to GREM1 with an antibody, or antigen-binding fragment thereof, comprising the complementarity determining regions (CDRs) of a heavy chain variable region (HCVR), wherein the HCVR has an amino acid sequence selected from the group consisting of SEQ ID NOs: 2, 18, 34, 50, 66, 82, 98, 1 14, 130, 146, 162, 178, 194, 210, 226, 242, 258, 274, 290, 306, 322, 338, 354, 370, 386, 402, 418, 434, 450, 466, 482, 498, 514, 530, 546, 562, and 578. In yet another embodiment, the antibody, or antigen-binding fragment thereof, competes for specific binding to GREM1 with an antibody, or antigen-binding fragment thereof, comprising the CDRs of a light chain variable region (LCVR), wherein the LCVR has an amino acid sequence selected from the group consisting of SEQ ID NOs: 10, 26, 42, 58, 74, 90, 106, 122, 138, 154, 170, 186, 202, 218, 234, 250, 266, 282, 298, 314, 330, 346, 362, 378, 394, 410, 426, 442, 458, 474, 490, 506, 522, 538, 554, 570, and 586.

In one embodiment, the antibody, or antigen-binding fragment thereof, comprises three heavy chain complementarity determining regions (CDRs) (HCDR1, HCDR2 and HCDR3) contained within any one of the heavy chain variable region (HCVR) sequences selected from the group consisting of SEQ ID NOs: 2, 18, 34, 50, 66, 82, 98, 1 14, 130, 146, 162, 178, 194, 210, 226, 242, 258, 274, 290, 306, 322, 338, 354, 370, 386, 402, 418, 434, 450, 466, 482, 498, 514, 530, 546, 562, and 578; and three light chain CDRs (LCDR1, LCDR2 and LCDR3) contained within any one of the light chain variable region (LCVR) sequences selected from the group consisting of SEQ ID NOs: 10, 26, 42, 58, 74, 90, 106, 122, 138, 154, 170, 186, 202, 218, 234, 250, 266, 282, 298, 314, 330, 346, 362, 378, 394, 410, 426, 442, 458, 474, 490, 506, 522, 538, 554, 570, and 586, e.g., the antibody, or antigen-binding fragment thereof, comprises a HCVR having an amino acid sequence selected from the group consisting of SEQ ID NOs: 2, 18, 34, 50, 66, 82, 98, 1 14, 130, 146, 162, 178, 194, 210, 226, 242, 258, 274, 290, 306, 322, 338, 354, 370, 386, 402, 418, 434, 450, 466, 482, 498, 514, 530, 546, 562, and 578; and/or the antibody, or antigen-binding fragment thereof, comprises a LCVR having an amino acid sequence selected from the group consisting of SEQ ID NOs: 10, 26, 42, 58, 74, 90, 106, 122, 138, 154, 170, 186, 202, 218, 234, 250, 266, 282, 298, 314, 330, 346, 362, 378, 394, 410, 426, 442, 458, 474, 490, 506, 522, 538, 554, 570, and 586; and/or the antibody, or antigen-binding fragment thereof, comprises: (a) a HCVR having an amino acid sequence selected from the group consisting of SEQ ID NOs: 2, 18, 34, 50, 66, 82, 98, 1 14, 130, 146, 162, 178, 194, 210, 226, 242, 258, 274, 290, 306, 322, 338, 354, 370, 386, 402, 418, 434, 450, 466, 482, 498, 514, 530, 546, 562, and 578; and (b) a LCVR having an amino acid sequence selected from the group consisting of SEQ ID NO: 10, 26, 42, 58, 74, 90, 106, 122, 138, 154, 170, 186, 202, 218, 234, 250, 266, 282, 298, 314, 330, 346, 362, 378, 394, 410, 426, 442, 458, 474, 490, 506, 522, 538, 554, 570, and 586.

In another embodiment, the antibody, or antigen-binding fragment thereof, comprises

(a) a HCDR1 domain having an amino acid sequence selected from the group consisting of SEQ ID NOs: 4, 20, 36, 52, 68, 84, 100, 1 16, 132, 148, 164, 180, 196, 212, 228, 244, 260, 276, 292, 308, 324, 340, 356, 372, 388, 404, 420, 436, 452, 468, 484, 500, 516, 532, 548, 564, and 580;

(b) a HCDR2 domain having an amino acid sequence selected from the group consisting of SEQ ID NOs: 6, 22, 38, 54, 70, 86, 102, 1 18, 134, 150, 166, 182, 198, 214, 230, 246, 262, 278, 294, 310, 326, 342, 358, 374, 390, 406, 422, 438, 454, 470, 486, 502, 518, 534, 550, 566, and 582;

(c) a HCDR3 domain having an amino acid sequence selected from the group consisting of SEQ ID NOs: 8, 24, 40, 56, 72, 88, 104, 120, 136, 152, 168, 184, 200, 216, 232, 248, 264, 280, 296, 312, 328, 344, 360, 376, 392, 408, 424, 440, 456, 472, 488, 504, 520, 536, 552, 568, and 584;

(d) a LCDR1 domain having an amino acid sequence selected from the group consisting of SEQ ID NOs: 12, 28, 44, 60, 76, 92, 108, 124, 140, 156, 172, 188, 204, 220, 236, 252, 268, 284, 300, 316, 332, 348, 364, 380, 396, 412, 428, 444, 460, 476, 492, 508, 524, 540, 556, 572, and 588;

(e) a LCDR2 domain having an amino acid sequence selected from the group consisting of SEQ ID NOs: 14, 30, 46, 62, 78, 94, 1 10, 126, 142, 158, 174, 190, 206, 222, 238, 254, 270, 286, 302, 318, 334, 350, 366, 382, 398, 414, 430, 446, 462, 478, 494, 510, 526, 542, 558, 574, and 590; and/or

(f) a LCDR3 domain having an amino acid sequence selected from the group consisting of SEQ ID NOs: 16, 32, 48, 64, 80, 96, 1 12, 128, 144, 160, 176, 192, 208, 224, 240, 256, 272, 288, 304, 320, 336, 352, 368, 384, 400, 416, 432, 448, 464, 480, 496, 512, 528, 544, 560, 576, and 592.

In yet another embodiment, the antibody, or antigen-binding fragment thereof, comprises a HCVR/LCVR amino acid sequence pair selected from the group consisting of SEQ ID NOs: 2/10, 18/26, 34/42, 50/58, 66/74, 82/90, 98/106, 1 14/122, 130/138, 146/154, 162/170, 178/186, 194/202, 210/218, 226/234, 242/250, 258/266, 274/282, 290/298, 306/314, 322/330, 338/346, 354/362, 370/378, 386/394, 402/410, 418/426, 434/442, 450/458, 466/474, 482/490, 498/506, 514/522, 530/538, 546/554, 562/570, and 578/586.

In yet another embodiment, the antibody, or antigen-binding fragment thereof, binds the same epitope on GREM1 as an antibody or antigen-binding fragment comprising the complementarity determining regions (CDRs) of a heavy chain variable region (HCVR), wherein the HCVR has an amino acid sequence selected from the group consisting of SEQ ID NOs: 2, 18, 34, 50, 66, 82, 98, 1 14, 130, 146, 162, 178, 194, 210, 226, 242, 258, 274, 290, 306, 322, 338, 354, 370, 386, 402, 418, 434, 450, 466, 482, 498, 514, 530, 546, 562, and 578; and the CDRs of a light chain variable region (LCVR), wherein the LCVR has an amino acid sequence selected from the group consisting of SEQ ID NOs: 10, 26, 42, 58, 74, 90, 106, 122, 138, 154, 170, 186, 202, 218, 234, 250, 266, 282, 298, 314, 330, 346, 362, 378, 394, 410, 426, 442, 458, 474, 490, 506, 522, 538, 554, 570, and 586.

In other embodiments, the antibodies, or antigen-binding fragments thereof, suitable for use in the present invention are fully human monoclonal antibodies, or antigen-binding fragments thereof, that bind to human GREM1, wherein the antibodies, or fragments thereof exhibit one or more of the following characteristics: (i) comprises a HCVR having an amino acid sequence selected from the group consisting of SEQ ID NO: 2, 18, 34, 50, 66, 82, 98, 1 14, 130, 146, 162, 178, 194, 210, 226, 242, 258, 274, 290, 306, 322, 338, 354, 370, 386, 402, 418, 434, 450, 466, 482, 498, 514, 530, 546, 562, and 578, or a substantially similar sequence thereof having at least 90%, at least 95%, at least 98% or at least 99% sequence identity; (ii) comprises a LCVR having an amino acid sequence selected from the group consisting of SEQ ID NO: 10, 26, 42, 58, 74, 90, 106, 122, 138, 154, 170, 186, 202, 218, 234, 250, 266, 282, 298, 314, 330, 346, 362, 378, 394, 410, 426, 442, 458, 474, 490, 506, 522, 538, 554, 570, and 586, or a substantially similar sequence thereof having at least 90%, at least 95%, at least 98% or at least 99% sequence identity; (iii) comprises a HCDR3 domain having an amino acid sequence selected from the group consisting of SEQ ID NO: 8, 24, 40, 56, 72, 88, 104, 120, 136, 152, 168, 184, 200, 216, 232, 248, 264, 280, 296, 312, 328, 344, 360, 376, 392, 408, 424, 440, 456, 472, 488, 504, 520, 536, 552, 568, and 584, or a substantially similar sequence thereof having at least 90%, at least 95%, at least 98% or at least 99% sequence identity; and a LCDR3 domain having an amino acid sequence selected from the group consisting of SEQ ID NO: 16, 32, 48, 64, 80, 96, 1 12, 128, 144, 160, 176, 192, 208, 224, 240, 256, 272, 288, 304, 320, 336, 352, 368, 384, 400, 416, 432, 448, 464, 480, 496, 512, 528, 544, 560, 576, and 592, or a substantially similar sequence thereof having at least 90%, at least 95%, at least 98% or at least 99% sequence identity; (iv) comprises a HCDR1 domain having an amino acid sequence selected from the group consisting of SEQ ID NO: 4, 20, 36, 52, 68, 84, 100, 1 16, 132, 148, 164, 180, 196, 212, 228, 244, 260, 276, 292, 308, 324, 340, 356, 372, 388, 404, 420, 436, 452, 468, 484, 500, 516, 532, 548, 564, and 580, or a substantially similar sequence thereof having at least 90%, at least 95%, at least 98% or at least 99% sequence identity; a HCDR2 domain having an amino acid sequence selected from the group consisting of SEQ ID NO: 6, 22, 38, 54, 70, 86, 102, 1 18, 134, 150, 166, 182, 198, 214, 230, 246, 262, 278, 294, 310, 326, 342, 358, 374, 390, 406, 422, 438, 454, 470, 486, 502, 518, 534, 550, 566, and 582, or a substantially similar sequence thereof having at least 90%, at least 95%, at least 98% or at least 99% sequence identity; a LCDR1 domain having an amino acid sequence selected from the group consisting of SEQ ID NO: 12, 28, 44, 60, 76, 92, 108, 124, 140, 156, 172, 188, 204, 220, 236, 252, 268, 284, 300, 316, 332, 348, 364, 380, 396, 412, 428, 444, 460, 476, 492, 508, 524, 540, 556, 572, and 588, or a substantially similar sequence thereof having at least 90%, at least 95%, at least 98% or at least 99% sequence identity; and a LCDR2 domain having an amino acid sequence selected from the group consisting of SEQ ID NO: 14, 30, 46, 62, 78, 94, 1 10, 126, 142, 158, 174, 190, 206, 222, 238, 254, 270, 286, 302, 318, 334, 350, 366, 382, 398, 414, 430, 446, 462, 478, 494, 510, 526, 542, 558, 574, and 590, or a substantially similar sequence thereof having at least 90%, at least 95%, at least 98% or at least 99% sequence identity; (v) binds to GREM1 with a KD equal to or less than 10⁻⁷; (vi) blocks GREM1 binding to one of BMP2, BMP4 or BMP7; (vii) blocks GREM1 inhibition of BMP signaling and promotes cell differentiation; and (viii) blocks GREM1 binding to heparin.

In one embodiment, an isolated human antibody or antigen-binding fragment thereof suitable for use in the methods of the invention binds to GREM1 with a KD equal to or less than 10⁻⁷ M as measured by surface plasmon resonance.

In one embodiment, the isolated human antibody or antigen-binding fragment thereof which binds to GREM1 for use in the methods of the invention comprises three heavy chain complementarity determining regions (CDRs) (HCDR1, HCDR2 and HCDR3) contained within any one of the heavy chain variable region (HCVR) sequences selected from the group consisting of SEQ ID NOs: 2, 18, 34, 50, 66, 82, 98, 1 14, 130, 146, 162, 178, 194, 210, 226, 242, 258, 274, 290, 306, 322, 338, 354, 370, 386, 402, 418, 434, 450, 466, 482, 498, 514, 530, 546, 562, and 578; and three light chain CDRs (LCDR1, LCDR2 and LCDR3) contained within any one of the light chain variable region (LCVR) sequences selected from the group consisting of SEQ ID NOs: 10, 26, 42, 58, 74, 90, 106, 122, 138, 154, 170, 186, 202, 218, 234, 250, 266, 282, 298, 314, 330, 346, 362, 378, 394, 410, 426, 442, 458, 474, 490, 506, 522, 538, 554, 570, and 586.

In one embodiment, the methods of the present invention include the use of an isolated human antibody or antigen-binding fragment thereof which binds to GREM1 and comprises a HCVR/LCVR amino acid sequence pair selected from the group consisting of SEQ ID NOs: 2/10, 18/26, 34/42, 50/58, 66/74, 82/90, 98/106, 114/122, 130/138, 146/154, 162/170, 178/186, 194/202, 210/218, 226/234, 242/250, 258/266, 274/282, 290/298, 306/314, 322/330, 338/346, 354/362, 370/378, 386/394, 402/410, 418/426, 434/442, 450/458, 466/474, 482/490, 498/506, 514/52, 530/538, 546/554, 562/570, and 578/586.

In another embodiment, the methods of the present invention include the use of an isolated human antibody or antigen-binding fragment thereof which binds to GREM1, wherein the antibody or fragment thereof exhibits one or more of the following characteristics: (i) comprises a HCVR having an amino acid sequence selected from the group consisting of SEQ ID NO: 2, 18, 34, 50, 66, 82, 98, 1 14, 130, 146, 162, 178, 194, 210, 226, 242, 258, 274, 290, 306, 322, 338, 354, 370, 386, 402, 418, 434, 450, 466, 482, 498, 514, 530, 546, 562, and 578, or a substantially similar sequence thereof having at least 90%, at least 95%, at least 98% or at least 99% sequence identity; (ii) comprises a LCVR having an amino acid sequence selected from the group consisting of SEQ ID NO: 10, 26, 42, 58, 74, 90, 106, 122, 138, 154, 170, 186, 202, 218, 234, 250, 266, 282, 298, 314, 330, 346, 362, 378, 394, 410, 426, 442, 458, 474, 490, 506, 522, 538, 554, 570, and 586, or a substantially similar sequence thereof having at least 90%, at least 95%, at least 98% or at least 99% sequence identity; (iii) comprises a HCDR3 domain having an amino acid sequence selected from the group consisting of SEQ ID NO: 8, 24, 40, 56, 72, 88, 104, 120, 136, 152, 168, 184, 200, 216, 232, 248, 264, 280, 296, 312, 328, 344, 360, 376, 392, 408, 424, 440, 456, 472, 488, 504, 520, 536, 552, 568, and 584, or a substantially similar sequence thereof having at least 90%, at least 95%, at least 98% or at least 99% sequence identity; and a LCDR3 domain having an amino acid sequence selected from the group consisting of SEQ ID NO: 16, 32, 48, 64, 80, 96, 1 12, 128, 144, 160, 176, 192, 208, 224, 240, 256, 272, 288, 304, 320, 336, 352, 368, 384, 400, 416, 432, 448, 464, 480, 496, 512, 528, 544, 560, 576, and 592, or a substantially similar sequence thereof having at least 90%, at least 95%, at least 98% or at least 99% sequence identity; (iv) comprises a HCDR1 domain having an amino acid sequence selected from the group consisting of SEQ ID NO: 4, 20, 36, 52, 68, 84, 100, 1 16, 132, 148, 164, 180, 196, 212, 228, 244, 260, 276, 292, 308, 324, 340, 356, 372, 388, 404, 420, 436, 452, 468, 484, 500, 516, 532, 548, 564, and 580, or a substantially similar sequence thereof having at least 90%, at least 95%, at least 98% or at least 99% sequence identity; a HCDR2 domain having an amino acid sequence selected from the group consisting of SEQ ID NO: 6, 22, 38, 54, 70, 86, 102, 1 18, 134, 150, 166, 182, 198, 214, 230, 246, 262, 278, 294, 310, 326, 342, 358, 374, 390, 406, 422, 438, 454, 470, 486, 502, 518, 534, 550, 566, and 582, or a substantially similar sequence thereof having at least 90%, at least 95%, at least 98% or at least 99% sequence identity; a LCDR1 domain having an amino acid sequence selected from the group consisting of SEQ ID NO: 12, 28, 44, 60, 76, 92, 108, 124, 140, 156, 172, 188, 204, 220, 236, 252, 268, 284, 300, 316, 332, 348, 364, 380, 396, 412, 428, 444, 460, 476, 492, 508, 524, 540, 556, 572, and 588, or a substantially similar sequence thereof having at least 90%, at least 95%, at least 98% or at least 99% sequence identity; and a LCDR2 domain having an amino acid sequence selected from the group consisting of SEQ ID NO: 14, 30, 46, 62, 78, 94, 1 10, 126, 142, 158, 174, 190, 206, 222, 238, 254, 270, 286, 302, 318, 334, 350, 366, 382, 398, 414, 430, 446, 462, 478, 494, 510, 526, 542, 558, 574, and 590, or a substantially similar sequence thereof having at least 90%, at least 95%, at least 98% or at least 99% sequence identity; (v) binds to GREM1 with a KD equal to or less than 10⁻⁷ M as measured by surface plasmon resonance.

In yet another embodiment, the methods of the present invention include the use of an isolated human antibody or antigen-binding fragment thereof which binds to GREM1 and comprises the complementarity determining regions (CDRs) of a heavy chain variable region (HCVR), wherein the HCVR has an amino acid sequence selected from the group consisting of SEQ ID NOs: 2, 18, 34, 50, 66, 82, 98, 1 14, 130, 146, 162, 178, 194, 210, 226, 242, 258, 274, 290, 306, 322, 338, 354, 370, 386, 402, 418, 434, 450, 466, 482, 498, 514, 530, 546, 562, and 578; and the CDRs of a light chain variable region (LCVR), wherein the LCVR has an amino acid sequence selected from the group consisting of SEQ ID NOs: 10, 26, 42, 58, 74, 90, 106, 122, 138, 154, 170, 186, 202, 218, 234, 250, 266, 282, 298, 314, 330, 346, 362, 378, 394, 410, 426, 442, 458, 474, 490, 506, 522, 538, 554, 570, and 586.

In one embodiment, the invention provides methods which include the use of an isolated antibody or antigen-binding fragment thereof that binds the same epitope on human GREM1 as an antibody or antigen-binding fragment comprising the CDRs of a heavy chain variable region (HCVR), wherein the HCVR has an amino acid sequence selected from the group consisting of SEQ ID NOs: 2, 18, 34, 50, 66, 82, 98, 1 14, 130, 146, 162, 178, 194, 210, 226, 242, 258, 274, 290, 306, 322, 338, 354, 370, 386, 402, 418, 434, 450, 466, 482, 498, 514, 530, 546, 562, and 578; and the CDRs of a light chain variable region (LCVR), wherein the LCVR has an amino acid sequence selected from the group consisting of SEQ ID NOs: 10, 26, 42, 58, 74, 90, 106, 122, 138, 154, 170, 186, 202, 218, 234, 250, 266, 282, 298, 314, 330, 346, 362, 378, 394, 410, 426, 442, 458, 474, 490, 506, 522, 538, 554, 570, and 586.

In one embodiment, the methods of the present invention include the use of an isolated human antibody or antigen-binding fragment thereof which blocks binding of human GREM1 to any one of BMP2, BMP4, BMP7 or heparin, the antibody comprising the complementarity determining regions (CDRs) of a heavy chain variable region (HCVR), wherein the HCVR has an amino acid sequence selected from the group consisting of SEQ ID NOs: 2, 18, 34, 50, 66, 82, 98, 1 14, 130, 146, 162, 178, 194, 210, 226, 242, 258, 274, 290, 306, 322, 338, 354, 370, 386, 402, 418, 434, 450, 466, 482, 498, 514, 530, 546, 562, and 578; and the CDRs of a light chain variable region (LCVR), wherein the LCVR has an amino acid sequence selected from the group consisting of SEQ ID NOs: 10, 26, 42, 58, 74, 90, 106, 122, 138, 154, 170, 186, 202, 218, 234, 250, 266, 282, 298, 314, 330, 346, 362, 378, 394, 410, 426, 442, 458, 474, 490, 506, 522, 538, 554, 570, and 586.

In another embodiment, the invention includes the use of a fully human monoclonal antibody or antigen-binding fragment thereof that binds to GREM1, wherein the antibody or fragment thereof exhibits one or more of the following characteristics: (i) comprises a HCVR having an amino acid sequence selected from the group consisting of SEQ ID NO: 2, 18, 34, 50, 66, 82, 98, 1 14, 130, 146, 162, 178, 194, 210, 226, 242, 258, 274, 290, 306, 322, 338, 354, 370, 386, 402, 418, 434, 450, 466, 482, 498, 514, 530, 546, 562, and 578, or a substantially similar sequence thereof having at least 90%, at least 95%, at least 98% or at least 99% sequence identity; (ii) comprises a LCVR having an amino acid sequence selected from the group consisting of SEQ ID NO: 10, 26, 42, 58, 74, 90, 106, 122, 138, 154, 170, 186, 202, 218, 234, 250, 266, 282, 298, 314, 330, 346, 362, 378, 394, 410, 426, 442, 458, 474, 490, 506, 522, 538, 554, 570, and 586, or a substantially similar sequence thereof having at least 90%, at least 95%, at least 98% or at least 99% sequence identity; (iii) comprises a HCDR3 domain having an amino acid sequence selected from the group consisting of SEQ ID NO: 8, 24, 40, 56, 72, 88, 104, 120, 136, 152, 168, 184, 200, 216, 232, 248, 264, 280, 296, 312, 328, 344, 360, 376, 392, 408, 424, 440, 456, 472, 488, 504, 520, 536, 552, 568, and 584, or a substantially similar sequence thereof having at least 90%, at least 95%, at least 98% or at least 99% sequence identity; and a LCDR3 domain having an amino acid sequence selected from the group consisting of SEQ ID NO: 16, 32, 48, 64, 80, 96, 1 12, 128, 144, 160, 176, 192, 208, 224, 240, 256, 272, 288, 304, 320, 336, 352, 368, 384, 400, 416, 432, 448, 464, 480, 496, 512, 528, 544, 560, 576, and 592, or a substantially similar sequence thereof having at least 90%, at least 95%, at least 98% or at least 99% sequence identity; (iv) comprises a HCDR1 domain having an amino acid sequence selected from the group consisting of SEQ ID NO: 4, 20, 36, 52, 68, 84, 100, 1 16, 132, 148, 164, 180, 196, 212, 228, 244, 260, 276, 292, 308, 324, 340, 356, 372, 388, 404, 420, 436, 452, 468, 484, 500, 516, 532, 548, 564, and 580, or a substantially similar sequence thereof having at least 90%, at least 95%, at least 98% or at least 99% sequence identity; a HCDR2 domain having an amino acid sequence selected from the group consisting of SEQ ID NO: 6, 22, 38, 54, 70, 86, 102, 1 18, 134, 150, 166, 182, 198, 214, 230, 246, 262, 278, 294, 310, 326, 342, 358, 374, 390, 406, 422, 438, 454, 470, 486, 502, 518, 534, 550, 566, and 582, or a substantially similar sequence thereof having at least 90%, at least 95%, at least 98% or at least 99% sequence identity; a LCDR1 domain having an amino acid sequence selected from the group consisting of SEQ ID NO: 12, 28, 44, 60, 76, 92, 108, 124, 140, 156, 172, 188, 204, 220, 236, 252, 268, 284, 300, 316, 332, 348, 364, 380, 396, 412, 428, 444, 460, 476, 492, 508, 524, 540, 556, 572, and 588, or a substantially similar sequence thereof having at least 90%, at least 95%, at least 98% or at least 99% sequence identity; and a LCDR2 domain having an amino acid sequence selected from the group consisting of SEQ ID NO: 14, 30, 46, 62, 78, 94, 1 10, 126, 142, 158, 174, 190, 206, 222, 238, 254, 270, 286, 302, 318, 334, 350, 366, 382, 398, 414, 430, 446, 462, 478, 494, 510, 526, 542, 558, 574, and 590, or a substantially similar sequence thereof having at least 90%, at least 95%, at least 98% or at least 99% sequence identity; (v) binds to GREM1 with a KD equal to or less than 10⁻⁷ M as measured by surface plasmon resonance; (vi) blocks GREM1 binding to one of BMP2, BMP4 or BMP7; (vii) blocks GREM1-inhibition of BMP signaling and promotes cell differentiation; and (viii) blocks GREM1 binding to heparin.

In another embodiment, the invention provides methods which include the use of an antibody or fragment thereof comprising a HCVR encoded by a nucleic acid sequence selected from the group consisting of SEQ ID NO: 1, 17, 33, 49, 65, 81, 97, 1 13, 129, 145, 161, 177, 193, 209, 225, 241, 257, 273, 289, 305, 321, 337, 353, 369, 385, 401, 417, 433, 449, 465, 481, 497, 513, 529, 545, 561, and 577, or a substantially identical sequence having at least 90%, at least 95%, at least 98%, or at least 99% homology thereof.

In one embodiment, the antibody or fragment thereof further comprises a LCVR encoded by a nucleic acid sequence selected from the group consisting of SEQ ID NO: 9, 25, 41, 57, 73, 89, 105, 121, 137, 153, 169, 185, 201, 217, 233, 249, 265, 281, 297, 313, 329, 345, 361, 377, 393, 409, 425, 441, 457, 473, 489, 505, 521, 537, 553, 569, and 585, or a substantially identical sequence having at least 90%, at least 95%, at least 98%, or at least 99% homology thereof.

In one embodiment, the methods of the invention include the use of an antibody or antigen-binding fragment of an antibody comprising a HCDR3 domain encoded by a nucleotide sequence selected from the group consisting of SEQ ID NO: 7, 23, 39, 55, 71, 87, 103, 119, 135, 151, 167, 183, 199,215, 231, 247, 263, 279, 295, 311, 327, 343, 359, 375, 391, 407, 423, 439, 455, 471, 487, 503, 519, 535, 551, 567, and 583, or a substantially similar sequence thereof having at least 90%, at least 95%, at least 98% or at least 99% sequence identity; and a LCDR3 domain encoded by a nucleotide sequence selected from the group consisting of SEQ ID NO: 15, 31, 47, 63, 79, 95, 111, 127, 143, 159, 175, 191,207, 223, 239, 255, 271,287, 303,319,335, 351,367, 383, 399, 415, 431, 447, 463, 479, 495, 511, 527, 543, 559, 575, and 591, or a substantially similar sequence thereof having at least 90%, at least 95%, at least 98% or at least 99% sequence identity.

In another embodiment, the methods of the invention include the use of an antibody or fragment thereof further comprising a HCDR1 domain encoded by a nucleotide sequence selected from the group consisting of SEQ ID NO: 3, 19, 35, 51, 67, 83, 99, 115, 131, 147, 163, 179, 195,211,227, 243, 259, 275, 291, 307, 323, 339, 355, 371, 387, 403, 419, 435, 451, 467, 483, 499, 515, 531, 547, 563, and 579, or a substantially similar sequence thereof having at least 90%, at least 95%, at least 98% or at least 99% sequence identity; a HCDR2 domain encoded by a nucleotide sequence selected from the group consisting of SEQ ID NO: 5, 21, 37, 53, 69, 85, 101, 117, 133, 149, 165, 181, 197, 213, 229, 245, 261, 277, 293, 309, 325, 341, 357, 373, 389, 405, 421, 437, 453, 469, 485, 501, 517, 533, 549, 565, and 581, or a substantially similar sequence thereof having at least 90%, at least 95%, at least 98% or at least 99% sequence identity; a LCDR1 domain encoded by a nucleotide sequence selected from the group consisting of SEQ ID NO: 11, 27, 43, 59, 75, 91, 107, 123, 139, 155, 171, 187, 203,219, 235, 251,267, 283, 299, 315, 331, 347, 363, 379, 395, 411, 427, 443, 459, 475, 491, 507, 523, 539, 555, 571, and 587, or a substantially similar sequence thereof having at least 90%, at least 95%, at least 98% or at least 99% sequence identity; and a LCDR2 domain encoded by a nucleotide sequence selected from the group consisting of SEQ ID NO: 13, 29, 45, 61, 77, 93, 109, 125, 141, 157, 173, 189, 205, 221, 237, 253, 269, 285, 301, 317, 333, 349, 365, 381, 397, 413, 429, 445, 461, 477, 493, 509, 525, 541, 557, 573, and 589, or a substantially similar sequence thereof having at least 90%, at least 95%, at least 98% or at least 99% sequence identity.

### BRIEF DESCRIPTION OF THE DRAWINGS

FIGS. 1A and 1B are graphs demonstrating that administration of H4H6245P restores pulmonary artery size (cross-sectional area) and right ventricular stroke volumes to near normoxic levels in a chronic hypoxia mouse model of pulmonary arterial hypertension.

FIG. 1A is a graph depicting the effect of administration of REGN2477 on pulmonary artery (PA) cross-sectional area (CSA) in a chronic hypoxia mouse model of pulmonary arterial hypertension.

FIG. 1B is a graph depicting the effect of administration of REGN2477 on right ventricular stroke volume in a chronic hypoxia mouse model of pulmonary arterial hypertension.

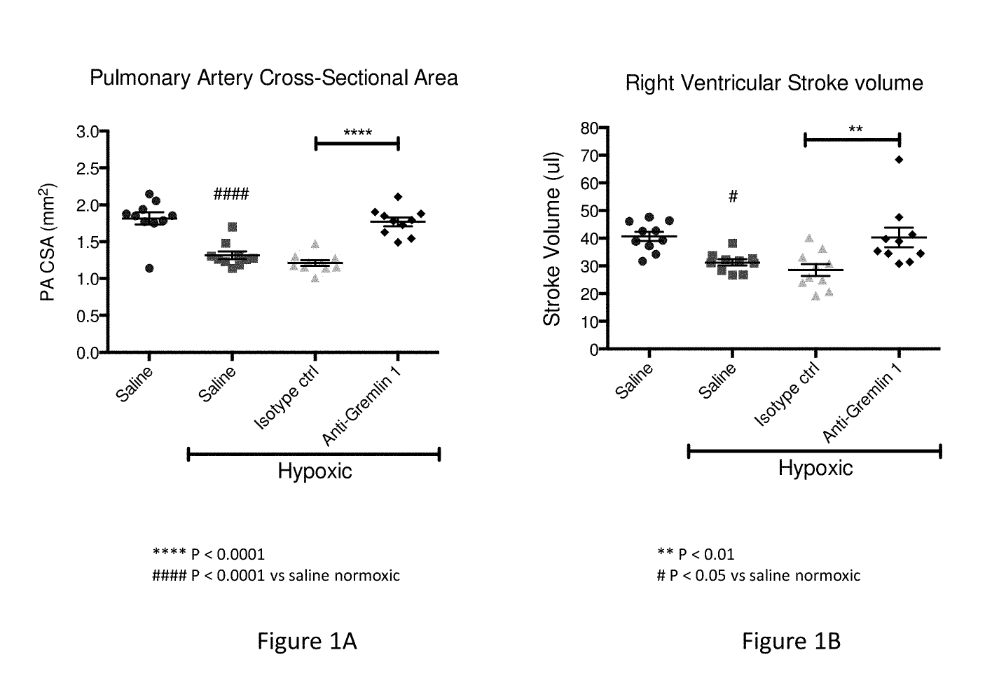

### DETAILED DESCRIPTION OF THE INVENTION

The present invention is based, at least in part, on the discovery that anti-GREM1 antibodies, or antigen-binding fragments thereof, are effective for ameliorating the effects of vascular remodeling in animal models of pulmonary arterial hypertension. The following detailed description discloses how to make and use compositions containing anti-GREM1 antibodies, or antigen-binding fragments thereof, to selectively inhibit the activity of GREM1 as well as compositions, uses, and methods for treating subjects having pulmonary arterial hypertension (PAH).

### I. Definitions

In order that the present invention may be more readily understood, certain terms are first defined. In addition, it should be noted that whenever a value or range of values of a parameter are recited, it is intended that values and ranges intermediate to the recited values are also intended to be part of this invention.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”.

The term “or” is used herein to mean, and is used interchangeably with, the term “and/or,” unless context clearly indicates otherwise.

The term “at least” prior to a number or series of numbers is understood to include the number adjacent to the term “at least”, and all subsequent numbers or integers that could logically be included, as clear from context. When at least is present before a series of numbers or a range, it is understood that “at least” can modify each of the numbers in the series or range.

As used herein, ranges include both the upper and lower limit.

The term “bone morphogenetic protein” or “BMP” refers to the group of growth factors which function as pivotal morphogenetic signals, orchestrating tissue architecture throughout the body. Originally discovered by their ability to induce the formation of bone and cartilage, BMPs are now known to have a variety of different functions during embryonic development, to be involved in body patterning and morphogenesis cascades, and to be essential in organ homeostasis. To date, twenty BMPs have been discovered, of which BMP2 to BMP7 belong to the transforming growth factor beta superfamily.

The term “GREM1” refers to human gremlin-1, a member of the cysteine knot superfamily. The amino acid sequence of human GREM1 is provided in GenBank as accession number NP_037504 and is also referred to herein as SEQ ID NO: 594. GREM1 is encoded by the nucleic acid provided herein as SEQ ID NO: 593, and is also found in GenBank as accession number NM_013372. GREM1 is a highly conserved 184 aa protein which has been mapped to chromosome 15q13-q15. The protein contains a signal peptide (aa 1-24), a predicted glycosylation site (at aa 42), a cysteine-rich region, and a cysteine knot motif (aa 94-184) whose structure is shared by members of the transforming growth factor-beta (TGF-β) superfamily. GREM1 exists in both secreted and cell-associated (e.g. membrane associated) forms. GREM1 is also known as gremlin 1, cysteine knot superfamily 1-BMP antagonist 1 (CKTSF1 B1), DAN domain family member 2 (DAND2), Down-regulated in Mos-transformed cells protein (DRM), gremlin, GREMLIN, Gremlin-1 precursor, Increased in high glucose protein 2 (IHG-2), MGC126660, Proliferation-inducing gene 2 protein (PIG2), or Gremlin 1-like protein. GREM1 is an antagonist of bone morphogenetic proteins (BMPs). It binds to BMPs and inhibits their binding to their receptors. The interplay between GREM1 and BMPs fine-tunes the level of available BMPs and affects developmental and disease processes. GREM1 can bind to and inhibit BMP-2, BMP-4 and BMP-7.

The term “pulmonary hypertension” (“PH”) is a term used to describe high blood pressure in the lungs from any cause. The terms “hypertension” or “high blood pressure,” on the other hand, refer to high blood pressure in the arteries throughout the body.

The term “pulmonary arterial hypertension” (“PAH”) refers to a progressive lung disorder which is characterized by sustained elevation of pulmonary artery pressure. Those patients with PAH typically have pulmonary artery pressure that is equal to or greater than 25 mm Hg with a pulmonary capillary or left atrial pressure equal to or less than 15 mm Hg. These pressures are typically measured in a subject at rest using right-heart catheterization. PAH, when untreated, leads to death (on average) within 2.8 years after being diagnosed.

The World Health Organization (WHO) has provided a clinical classification of PAH of five groups (Simonneau, et al. J Am Coll Cardiol. 2013; 62(25_S), the entire contents of which are incorporated herein by reference):

1. Pulmonary arterial hypertension (PAH)
1.1. Idiopathic
1.2. Heritable
   1.2.1. BMPR2
   1.2.2. ALK1, ENG, SMAD9, CAV1, KCNK3
   1.2.3. Unknown
1.3. Drug- and toxin-induced
1.4. Associated with:
   1.4.1. Connective tissue diseases
   1.4.2. HIV infection
   1.4.3. Portal Hypertension
   1.4.4. Congenital heart diseases
   1.4.5. Schistosomiasis
      1′. Pulmonary veno-occlusive disease (PVOD) and/or pulmonary capillary hemangiomatosis (PCH)
      1″. Persistent pulmonary hypertension of the newborn (PPHN)
      2. Pulmonary hypertension due to left heart disease
2.1. Left ventricular systolic dysfunction
2.2. Left ventricular diastolic dysfunction
2.3. Valvular disease
2.4. Congenital/acquired left heart inflow/outflow tract obstruction and congenital cardiomyopathies
   3. Pulmonary hypertension due to lung disease and/or hypoxia
3.1. Chronic obstructive pulmonary disease
3.2. Interstitial lung disease
3.3. Other pulmonary diseases with mixed restrictive and obstructive pattern
3.4. Sleep-disordered breathing
3.5. Alveolar hypoventilation disorders
3.6. Chronic exposure to high altitude
3.7. Developmental abnormalities
   4. Chronic thromboembolic pulmonary hypertension (CTEPH)
   5. Pulmonary hypertension with unclear multifactorial mechanisms
5.1. Hematologic disorders: chronic hemolytic anemia, myeloproliferative disorders, splenectomy
5.2. Systemic disorders: sarcoidosis, pulmonary histiocytosis, lymphangioleimoyomatosis
5.3. Metabolic disorders: glycogen storage disease, Gaucher disease, thyroid disorders
5.4. Others: tumoral obstruction, fibrosing mediastinitis, chronic renal failure on dialysis, segmental PH.

In one embodiment, a subject that would benefit from the methods of the present invention is a subject having Group I (WHO) PAH.

PAH at baseline (e.g., when diagnosed) can be mild, moderate or severe, as measured, for example, by the WHO functional class, which is a measure of disease severity in patients with PAH. The WHO functional classification is an adaptation of the New York Heart Association (NYHA) system and is routinely used to qualitatively assess activity tolerance, for example, in monitoring disease progression and response to treatment (Rubin (2004) Chest 126:7-10). There are four functional classes recognized in the WHO system:

Class I: pulmonary hypertension without resulting limitation of physical activity; ordinary physical activity does not cause undue dyspnea or fatigue, chest pain or near syncope;

Class II: pulmonary hypertension resulting in slight limitation of physical activity; patient comfortable at rest; ordinary physical activity causes undue dyspnea or fatigue, chest pain or near syncope;

Class III: pulmonary hypertension resulting in marked limitation of physical activity; patient comfortable at rest; less than ordinary activity causes undue dyspnea or fatigue, chest pain or near syncope; and Class IV: pulmonary hypertension resulting in inability to carry out any physical activity without symptoms; patient manifests signs of right-heart failure; dyspnea and/or fatigue may be present even at rest; discomfort is increased by any physical activity.

In one embodiment, a subject that would benefit from the methods of the present invention is a subject having, at baseline, PAH e.g., Group I (WHO) PAH) of WHO Class I. In another embodiment, a subject that would benefit from the methods of the present invention is a subject having, at baseline, PAH (e.g., Group I (WHO) PAH) of WHO Class II. In another embodiment, a subject that would benefit from the methods of the present invention is a subject having, at baseline, PAH e.g., Group I (WHO) PAH) of WHO Class III.

As used herein, a “subject” is an animal, such as a mammal, including a primate (such as a human, a non-human primate, e.g., a monkey, and a chimpanzee), a non-primate (such as a cow, a pig, a camel, a llama, a horse, a goat, a rabbit, a sheep, a hamster, a guinea pig, a cat, a dog, a rat, a mouse, a horse, and a whale), or a bird (e.g., a duck or a goose).

In one embodiment, the subject is a human, such as a human being treated or assessed for PAH e.g., Group I (WHO) PAH; a human at risk for PAH e.g., Group I (WHO) PAH; a human having PAH e.g., Group I (WHO) PAH; and/or human being treated for PAH e.g., Group I

(WHO) PA), as described herein.

As used herein, the terms “treating” or “treatment” refer to a beneficial or desired result including, but not limited to, alleviation or amelioration of one or more symptoms associated with PAH e.g., Group I (WHO) PAH). “Treatment” can also mean slowing the course of the disease or reducing the development of a symptom of disease, reducing the severity of later-developing disease, or prolonging survival as compared to expected survival in the absence of treatment. For example, the reduction in the development of a symptom associated with such a disease, disorder or condition (e.g., by at least about 10% on a clinically accepted scale for that disease or disorder), or the exhibition of delayed symptoms delayed (e.g., by days, weeks, months or years) is considered effective treatment.

“Therapeutically effective amount,” as used herein, is intended to include the amount of an anti-GREM1 antibody, or antigen-binding fragment thereof, that, when administered to a subject having PAH e.g., Group I (WHO) PAH, is sufficient to effect treatment of the disease (e.g., by diminishing, ameliorating or maintaining the existing disease or one or more symptoms of disease) or manage the disease. The “therapeutically effective amount” may vary depending on the anti-GREM1 antibody, or antigen-binding fragment thereof, how the anti-GREM1 antibody, or antigen-binding fragment thereof, is administered, the disease and its severity and the history, age, weight, family history, genetic makeup, stage of PAH, the types of preceding or concomitant treatments, if any, and other individual characteristics of the patient to be treated.

A “therapeutically effective amount” is also intended to include the amount of an anti-GREM1 antibody, or antigen-binding fragment thereof, that, when administered to a subject is sufficient to ameliorate the disease or one or more symptoms of the disease. Ameliorating the disease includes slowing the course of the disease or reducing the severity of later-developing disease.

A “therapeutically-effective amount” also includes an amount of an anti-GREM1 antibody, or antigen-binding fragment thereof, that produces some desired local or systemic effect at a reasonable benefit/risk ratio applicable to any treatment. Anti-GREM1 antibodies, or antigen-binding fragments thereof, employed in the methods of the present invention may be administered in a sufficient amount to produce a reasonable benefit/risk ratio applicable to such treatment.

### II. Methods of the Invention

The present invention provides methods for treating a subject having pulmonary arterial hypertension. The methods generally include administering to the subject a therapeutically effective amount of an anti-GREM1 antibody, or antigen-binding fragment thereof.

In some aspects of the present invention, administration of the anti-GREM1 antibody, or antigen-binding fragment thereof, inhibits thickening of the pulmonary artery in the subject, e.g., inhibit further thickening of the pulmonary artery in the subject from baseline, e.g., at diagnosis. The thickening of the pulmonary artery may be determined by, for example, chest CT (such as, unenhanced axial 10 mm CT sections), and used to calculate main pulmonary artery diameter (mPA). The main pulmonary artery diameter in normal subjects is about 2.4 cm to about 3.0 cm. Main pulmonary artery diameter in subjects with pulmonary arterial hypertension is about 3.1 cm to about 3.8 cm, or greater. See, e.g., Edwards, et al. (1998) Br J Radiol 71(850):1018-20.

In other aspects of the present invention, administration of the anti-GREM1 antibody, or antigen-binding fragment thereof, increases stroke volume and/or stroke volume to end systolic volume ratio (“SV/ESV”) in the subject. “Stroke volume” (“SV”) is the volume of blood pumped from the right or left ventricle per single contraction. Stroke volume may be calculated using measurements of ventricle volumes from an echocardiogram and calculated by subtracting the volume of the blood in the ventricle at the end of a beat (called “end-systolic volume,” “EDV”) from the volume of blood just prior to the beat (called “end-diastolic volume,” “ESV”). Stroke volume may also be calculated, e.g., as cardiac out put measured by thermodilution during right heart catheterization divided by heart rate or as EDV minus ESV and indexed for body surface area. The term stroke volume can apply to each of the two ventricles of the heart. The stroke volumes for each ventricle are generally equal, both being approximately 70 mL in a healthy subjects. The SV/ESV for healthy subjects is about 0.9 to about 2.2 and the SV/ESV for subjects having PAH is about 0.2 to about 0.9. See, e.g. Brewis, et al. (2016) Int J Cardiol 218:206-211.

In yet other aspects of the present invention, administration of the anti-GREM1 antibody, or antigen-binding fragment thereof, increases right ventricle cardiac output and/or cardiac index (CI) in the subject. “Cardiac output” (“CO”) is defined as the amount of blood pumped by a ventricle in unit time. “Cardiac index” (“CI”) is a haemodynamic parameter that relates the cardiac output (CO) from left ventricle in one minute to “body surface area” (“BSA”), thus relating heart performance to the size of the individual. Echocardiographic techniques and radionuclide imaging techniques can be used to estimate real-time changes in ventricular dimensions, thus computing stroke volume, which when multiplied by heart rate, gives cardiac output, and BSA may be calculated using any one of the formuals known to one of ordinary skill in the art including, for example, the Du Bois formula (Verbraecken, J, et al. (2006) Metabolism—Clin Exper 55(4):515-24) or the Mosteller formula (Mosteller (1987) N Engl J Med 317:1098). Subjects that do not have PAH have a cardiac output in the range of about 4.0-8.0 L/min and a cardiac index of about 2.6 toabout 4.2 L/minute per square meter. Subjects that have PAH have a cardiac index of about 1.9 to about 2.3 L/minute per square meter (Ryan and Archer (2016) Circ Res 115:176-188).

Administration of the anti-GREM1 antibody, or antigen-binding fragment thereof, to a subject having PAH in the methods of the present invention may improve other hemodynamic measurements in a subject having PAH, such as, for example, right atrium pressure, pulmonary artery pressure, pulmonary capillary wedge pressure in the presence of end expiratory pressure, systemic artery pressure, heart beat, pulmonary vascular resistance, and/or systemic vascular resistance. Methods and devices for measuring right atrium pressure, pulmonary artery pressure, pulmonary capillary wedge pressure in the presence of end expiratory pressure, systemic artery pressure, heart beat, pulmonary vascular resistance, and/or systemic vascular resistance are known to one of ordinary skill in the art.

Subjects that do not have PAH have a right atrium pressure of about 1 mm Hg to about 5 mm Hg; subjects that have PAH have a right atrium pressure of about 11 mm Hg to about 13 mm Hg.

Subjects that do not have PAH have a pulmonary artery pressure of about 9 mm Hg to about 20 mm Hg; subjects that have PAH have a pulmonary artery pressure of about 57 mm Hg to about 61 mm Hg.

Subjects that do not have PAH have a pulmonary capillary wedge pressure in the presence of end expiratory pressure of about 4 mm Hg to about 12 mm Hg; subjects that have PAH have a pulmonary capillary wedge pressure in the presence of end expiratory pressure of about 9 mm Hg to about 11 mm Hg.

Subjects that do not have PAH have a systemic artery pressure of about 90 mm Hg to about 96 mm Hg; subjects that have PAH have a systemic artery pressure of about 87 mm Hg to about 91 mm Hg.

Subjects that do not have PAH have a heart beat of about 60 beats per minute (bpm) to about 90 bpm; subjects that have PAH have a systemic artery pressure of about 84 bpm 88 bpm.

Subjects that do not have PAH have a pulmonary vascular resistance of about 20 dynes s/cm⁵ to about 130 dynes s/cm⁵ (or about 0.25 to about 1.625 wood units) subjects that have PAH have a pulmonary vascular resistance of about 1200 dynes s/cm⁵ to about 1360 dynes s/cm⁵ (or about 15 to about 17 wood units).

Subjects that do not have PAH have a systemic vascular resistance of about 700 dynes s/cm⁵ to about 1600 dynes s/cm⁵ (or about 9 to about 20 wood units) subjects that have PAH have a systemic vascular resistance of about 1840 dynes s/cm⁵ to about 2000 dynes s/cm⁵ (or about 23 to about 25 wood units).

The methods of the present invention may also improve other clinical parameters, such as pulmonary function, in the subject being treated. For example, during or following a treatment period a subject may have an increased exercise capacity or activity, as measured by, for example, a test of 6-minute walking distance (6 MWD) or measure of activity, or lowering Borg dyspnea index (BDI).

The methods of the present invention may also improve one or more quality of life parameters versus baseline, for example an increase in score on at least one of the SF-36® health survey functional scales; an improvement versus baseline in the severity of the condition, for example by movement to a lower WHO functional class; and/or an increased longevity.

Any suitable measure of exercise capacity can be used to determine whether a subject has an increased exercise capacity or activity. One suitable measure is a 6-minute walk test (6MWT), which measures how far the subject can walk in 6 minutes, i.e., the 6-minute walk distance (6MWD). Another suitable measure is the Borg dyspnea index (BDI), which is a numerical scale for assessing perceived dyspnea (breathing discomfort). It measures the degree of breathlessness after completion of the 6-minute walk test (6MWT), where a BDI of 0 indicates no breathlessness and 10 indicates maximum breathlessness. In one embodiment, the methods of the invention provide to the subject an increase from baseline in the 6MWD by at least about 10 minutes, e.g., about 10, 15, 20, or about 30 minutes. In another embodiment, following a 6MWT the methods of the invention provide to the subject a lower from baseline BDI by at least about 0.5 to about 1.0 index points.

Any suitable measure quality of life may be used. For example, the SF-36® health survey provides a self-reporting, multi-item scale measuring eight health parameters: physical functioning, role limitations due to physical health problems, bodily pain, general health, vitality (energy and fatigue), social functioning, role limitations due to emotional problems, and mental health (psychological distress and psychological well-being). The survey also provides a physical component summary and a mental component summary. In one embodiment, the methods of the invention provide to the subject an improvement versus baseline in at least one of the SF-36 physical health related parameters (physical health, role-physical, bodily pain and/or general health) and/or in at least one of the SF-36 mental health related parameters (vitality, social functioning, role-emotional and/or mental health). Such an improvement can take the form of an increase of at least 1, for example at least 2 or at least 3 points, on the scale for any one or more parameters.

The methods of the present invention may also improve the prognosis of the subject being treated. For example, the methods of the invention may provide to the subject a reduction in probability of a clinical worsening event during the treatment period, and/or a reduction from baseline in serum brain natriuretic peptide (BNP) or NT pro-BNP or its N-terminal prohormone, NT-pro-BNP concentration, wherein, at baseline, time from first diagnosis of the condition in the subject is not greater than about 2 years.

Time from first diagnosis, in various aspects, can be, for example, not greater than about 1.5 years, not greater than about 1 year, not greater than about 0.75 year, or not greater than about 0.5 year. A clinical worsening event (CWE) includes death, lung transplantation, hospitalization for the PAH, atrial septostomy, initiation of additional pulmonary hypertension therapy or a combination thereof. Time to clinical worsening of PAH is defined as the time from initiation of treatment to the first occurrence of a CWE.

In one embodiment, the methods of the invention provide a reduction from baseline of at least about 15%, for example at least about 25%, at least about 50% or at least about 75%, in BNP or NT-pro-BNP concentration.

In one embodiment, the methods of the invention provide a reduction of at least about 25%, for example at least about 50%, at least about 75%> or at least about 80%, in probability of death, lung transplantation, hospitalization for pulmonary arterial hypertension, atrial septostomy and/or initiation of additional pulmonary hypertension therapy during the treatment period.

The methods of the present invention may also prolong the life (extend survival time) of a subject having PAH, from a time of initiation of treatment by, for example, at least about 30 days.

The therapeutically effective amount of an anti-GREM1 antibody, or antigen-binding fragment thereof, for use in the methods of the invention may be from about 0.05 mg to about 600 mg; e.g., about 0.05 mg, about 0.1 mg, about 1.0 mg, about 1.5 mg, about 2.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, or about 1000 mg, of the respective antibody.

The amount of anti-GREM1 antibody, or antigen-binding fragment thereof, contained within an individual dose may be expressed in terms of milligrams of antibody per kilogram of patient body weight (i.e., mg/kg). For example, an anti-GREM1 antibody, or antigen-binding fragment thereof, may be administered to a patient at a dose of about 0.0001 to about 50 mg/kg of patient body weight (e.g. 0.1 mg/kg, 0.5 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0 mg/kg, 6.5 mg/kg, 7.0 mg/kg, 7.5 mg/kg, 8.0 mg/kg, 8.5 mg/kg, 9.0 mg/kg, 9.5 mg/kg, 10.0 mg/kg, 10.5 mg/kg, 11.0 mg/kg, 11.5 mg/kg, 12.0 mg/kg, 12.5 mg/kg, 13.0 mg/kg, 13.5 mg/kg, 14.0 mg/kg, 14.5 mg/kg, 15.0 mg/kg, 15.5 mg/kg, 16.0 mg/kg, 16.5 mg/kg, 17.0 mg/kg, 17.5 mg/kg, 18.0 mg/kg, 18.5 mg/kg, 19.0 mg/kg, 19.5 mg/kg, 20.0 mg/kg, etc.).

Multiple doses of an anti-GREM1 antibody, or antigen-binding fragment thereof, or a pharmaceutical composition comprising an anti-GREM1 antibody, or antigen-binding fragment thereof, may be administered to a subject over a defined time course. The methods according to this aspect of the invention comprise sequentially administering to a subject multiple doses of an active ingredient of the invention. As used herein, “sequentially administering” means that each dose of an active ingredient is administered to the subject at a different point in time, e.g., on different days separated by a predetermined interval (e.g., hours, days, weeks or months). The present invention includes methods which comprise sequentially administering to the patient a single initial dose of an active ingredient, followed by one or more secondary doses of the active ingredient, and optionally followed by one or more tertiary doses of the active ingredient.

The terms “initial dose,” “secondary doses,” and “tertiary doses,” refer to the temporal sequence of administration of an anti-GREM1 antibody, or antigen-binding fragment thereof, or of a combination therapy of the invention. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of anti-GREM1 antibody, or antigen-binding fragment thereof, but may differ from one another in terms of frequency of administration. In certain embodiments, however, the amount of anti-GREM1 antibody, or antigen-binding fragment thereof, contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In certain exemplary embodiments of the present invention, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose of an anti-GREM1 antibody, or antigen-binding fragment thereof, which is administered to a patient prior to the administration of the very next dose in the sequence with no intervening doses.

The methods according to this aspect of the invention may comprise administering to a patient any number of secondary and/or tertiary doses. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks or 1 to 2 months after the immediately preceding dose. Similarly, in embodiments involving multiple tertiary doses, each tertiary dose may be administered at the same frequency as the other tertiary doses. For example, each tertiary dose may be administered to the patient 2 to 12 weeks after the immediately preceding dose. In certain embodiments of the invention, the frequency at which the secondary and/or tertiary doses are administered to a patient can vary over the course of the treatment regimen. The frequency of administration may also be adjusted during the course of treatment by a physician, depending on the needs of the individual patient following clinical examination.

In some embodiment of the present invention, an anti-GREM1 antibody, or antigen-binding fragment thereof, may be administered as a monotherapy (i.e., as the only therapeutic agent). In other embodiments of the present invention, an anti-GREM1 antibody, or antigen-binding fragment thereof, may be administered in combination with one or more additional therapeutic agents.

In the combination methods of the invention which comprise administering an anti-GREM1 antibody, or antigen-binding fragment thereof, and at least one additional therapeutic agent to the subject, the antibody and the additional therapeutic agent may be administered to the subject at the same or substantially the same time, e.g., in a single therapeutic dosage, or in two separate dosages which are administered simultaneously or within less than about 5 minutes of one another. Alternatively, the antibody and the additional therapeutic agent may be administered to the subject sequentially, e.g., in separate therapeutic dosages separated in time from one another by more than about 5 minutes.

Accordingly, in one embodiment, the methods of the invention further comprise administering a therapeutically effective amount of at least one therapeutic agent selected from the group consisting of an anticoagulant, a diuretic, a cardiac glycoside, a calcium channel blocker, a vasodilator, a prostacyclin analogue, an endothelium antagonist, a phosphodiesterase inhibitor, an endopeptidase inhibitor, a lipid lowering agent, and a thromboxane inhibitor. In one embodiment, the methods of the invention further comprise administering a therapeutically effective amount of at least one or more additional therapeutic antibody or antibodies, or antigen-binding fragment or fragments thereof. In one embodiment, the one or more additional antibody or antibodies are selected from the group consisting of an anti-Grem 1 antibody or antibodies, an anti-PDGFRβ antibody or antibodies, an anti-TLR4 antibody or antibodies, an anti-TLR2 antibody or antibodies, an anti-EDN1 antibody or antibodies, and an anti-ASIC1 antibody orantibodies.

Examples of suitable anticoagulants include, but are not limited to, e.g. warfarin useful in the treatment of patients with pulmonary hypertension having an increased risk of thrombosis and thromboembolism.

Examples of suitable calcium channel blockers include, but are not limited to, diltiazem, felodipine, amlodipine and nifedipine.

Suitable vasodilators include, but are not limited to, e.g. prostacyclin, epoprostenol, treprostinil and nitric oxide (NO).

Suitable exemplary phosphodiesterase inhibitors include, but are not limited to, particularly phospho-diesterase V inhibitors such as e.g. tadalafil, sildenafil and vardenafil.

Examples of suitable endothelin antagonists include, but are not limited to, e.g. bosentan and sitaxentan.

Suitable prostacyclin analogues include, but are not limited to, e.g. ilomedin, treprostinil and epoprostenol.

Suitable lipid lowering agents include, but are not limited to, e.g. HMG CoA reductase inhibitors such as simvastatin, pravastatin, atorvastatin, lovastatin, itavastatin, fluvastatin, pitavastatin, rosuvastatin, ZD-4522 and cerivastatin

Diuretics suitable for use in the combination therapies of the invention include, but are not limited to, e.g. chlorthalidon, indapamid, bendro-flumethiazid, metolazon, cyclopenthiazid, polythiazid, mefrusid, ximapid, chlorothiazid and hydrochlorothiazid.

Examples of other therapeutics agents include, but are not limited to, e.g. ACE inhibitors such as enalapril, ramipril, captopril, cilazapril, trandolapril, fosinopril, quinapril, moexipril, lisinopril and perindopril, or ATII inhibitors such as losartan, candesartan, irbesartan, embusartan, valsartan and telmisartan, or iloprost, betaprost, L-arginine, omapatrilat, oxygen, and/or digoxin.

The methods of the invention may also include the combined use of kinase inhibitors (e.g., BMS-354825, canertinib, erlotinib, gefitinib, imatinib, lapatinib, lestaurtinib, lonafarnib, pegaptanib, pelitinib, semaxanib, tandutinib, tipifarnib, vatalanib, lonidamine, fasudil, leflunomide, bortezomib, imatinib, erlotinib and glivec) and/or elastase inhibitors.

The additional therapeutically active component(s) may be administered to a subject prior to administration of an anti-GREM1 antibody of the present invention. For example, a first component may be deemed to be administered “prior to” a second component if the first component is administered 1 week before, 72 hours before, 60 hours before, 48 hours before, 36 hours before, 24 hours before, 12 hours before, 6 hours before, 5 hours before, 4 hours before, 3 hours before, 2 hours before, 1 hour before, 30 minutes before, 15 minutes before, 10 minutes before, 5 minutes before, or less than 1 minute before administration of the second component. In other embodiments, the additional therapeutically active component(s) may be administered to a subject after administration of an anti-GREM1 antibody, or antigen-binding fragment thereof. For example, a first component may be deemed to be administered “after” a second component if the first component is administered 1 minute after, 5 minutes after, 10 minutes after, 15 minutes after, 30 minutes after, 1 hour after, 2 hours after, 3 hours after, 4 hours after, 5 hours after, 6 hours after, 12 hours after, 24 hours after, 36 hours after, 48 hours after, 60 hours after, 72 hours after administration of the second component.

In yet other embodiments, the additional therapeutically active component(s) may be administered to a subject concurrent with administration of anti-GREM1 antibody, or antigen-binding fragment thereof, of the present invention. “Concurrent” administration, for purposes of the present invention, includes, e.g., administration of an anti-GREM1 antibody and an additional therapeutically active component to a subject in a single dosage form, or in separate dosage forms administered to the subject within about 30 minutes or less of each other. If administered in separate dosage forms, each dosage form may be administered via the same route (e.g., both the anti-GREM1 antibody and the additional therapeutically active component may be administered intravenously, subcutaneously, intravitreally, etc.); alternatively, each dosage form may be administered via a different route (e.g., the anti-GREM1 antibody may be administered locally (e.g., intravitreally) and the additional therapeutically active component may be administered systemically). In any event, administering the components in a single dosage from, in separate dosage forms by the same route, or in separate dosage forms by different routes are all considered “concurrent administration,” for purposes of the present disclosure. For purposes of the present disclosure, administration of an anti-GREM1 antibody “prior to,” “concurrent with,” or “after” (as those terms are defined herein above) administration of an additional therapeutically active component is considered administration of an anti-GREM1 antibody, or antigen-binding fragment thereof, “in combination with” an additional therapeutically active component).

### III. Binding Proteins Suitable for Use in the Methods of the Invention

Suitable anti-gremlin-1 (GREM1) binding proteins for use in the methods of the present invention are described in, for example, U.S. Patent Publication No. 2016/0024195, the entire contents of which are incorporated herein by reference.

In one embodiment, a GREM1 binding protein suitable for use in the present invention is an antigen-specific binding protein.

As used herein, the expression “antigen-specific binding protein” means a protein comprising at least one domain which specifically binds a particular antigen. Exemplary categories of antigen-specific binding proteins include antibodies, antigen-binding portions of antibodies, peptides that specifically interact with a particular antigen (e.g., peptibodies), receptor molecules that specifically interact with a particular antigen, and proteins comprising a ligand-binding portion of a receptor that specifically binds a particular antigen.

Thus, the present invention includes the use of antigen-specific binding proteins that specifically bind GREM1, i.e., “GREM1-specific binding proteins.”

In one embodiment, an antigen-specific binding protein for use in the methods of the present invention may comprise or consist of an antibody or antigen-binding fragment of an antibody.

In one embodiment, a GREM1-specific binding protein for use in the present invention is a human monoclonal antibody that specifically binds to GREM1 of SEQ ID NO: 594 or SEQ ID NO: 595.

The term “antibody”, as used herein, is intended to refer to immunoglobulin molecules comprised of four polypeptide chains, two heavy (H) chains and two light (L) chains interconnected by disulfide bonds (i.e., “full antibody molecules”), as well as multimers thereof (e.g. IgM) or antigen-binding fragments thereof. Each heavy chain is comprised of a heavy chain variable region (“HCVR” or “V_{H}”) and a heavy chain constant region (comprised of domains C_{H}1, C_{H}2 and C_{H}3). Each light chain is comprised of a light chain variable region (“LCVR or “V_{L}”) and a light chain constant region (C_{L}). The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In certain embodiments of the invention, the FRs of the antibody (or antigen binding fragment thereof) may be identical to the human germline sequences, or may be naturally or artificially modified. An amino acid consensus sequence may be defined based on a side-by-side analysis of two or more CDRs.

Methods and techniques for identifying CDRs within HCVR and LCVR amino acid sequences are well known in the art and can be used to identify CDRs within the specified heavy chain variable region(s) (HCVR) and/or light chain variable region(s) (LCVR) amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., (1997), J. Mol. Biol. 273:927-948; and Martin et al., (1989), Proc. Natl. Acad. Sci. USA 86:9268-9272. Public databases are also available for identifying CDR sequences within an antibody.

Substitution of one or more CDR residues or omission of one or more CDRs is also possible. Antibodies have been described in the scientific literature in which one or two CDRs can be dispensed with for binding. Padlan et al. (FASEB J. 1995, 9:133-139) analyzed the contact regions between antibodies and their antigens, based on published crystal structures, and concluded that only about one fifth to one third of CDR residues actually contact the antigen. Padlan also found many antibodies in which one or two CDRs had no amino acids in contact with an antigen (see also, Vajdos et al. 2002 J Mol Biol 320:415-428).

CDR residues not contacting antigen can be identified based on previous studies (for example residues H60-H65 in CDRH2 are often not required), from regions of Kabat CDRs lying outside Chothia CDRs, by molecular modeling and/or empirically. If a CDR or residue(s) thereof is omitted, it is usually substituted with an amino acid occupying the corresponding position in another human antibody sequence or a consensus of such sequences. Positions for substitution within CDRs and amino acids to substitute can also be selected empirically. Empirical substitutions can be conservative or non-conservative substitutions.

The fully human anti-GREM1 monoclonal antibodies for use in the methods disclosed herein may comprise one or more amino acid substitutions, insertions and/or deletions in the framework and/or CDR regions of the heavy and light chain variable domains as compared to the corresponding germline sequences. Such mutations can be readily ascertained by comparing the amino acid sequences disclosed herein to germline sequences available from, for example, public antibody sequence databases. The present invention includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from whifch the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present invention may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present invention.

The present invention also includes use of fully human anti-GREM1 monoclonal antibodies comprising variants of any of the HCVR, LCVR, and/or CDR amino acid sequences disclosed herein having one or more conservative substitutions. For example, the present invention includes anti-GREM1 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

The term “human antibody,” as used herein, is intended to include antibodies having variable and constant regions derived from human germline immunoglobulin sequences. The human mAbs of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include mAbs in which CDR sequences derived from the germline of another mammalian species (e.g., mouse), have been grafted onto human FR sequences.

The term “specifically binds,” or “binds specifically to,” or the like, means that an antibody or antigen-binding fragment thereof, forms a complex with an antigen that is relatively stable under physiologic conditions. Specific binding can be characterized by an equilibrium dissociation constant of at least about 1×10⁻⁶ M or less (e.g., a smaller K_{D} denotes a tighter binding). Methods for determining whether two molecules specifically bind are well known in the art and include, for example, equilibrium dialysis, surface plasmon resonance, and the like. Suitable antibodies that bind specifically to human GREM1 for use herein have been identified by surface plasmon resonance, e.g., BIACORE™. Moreover, multi-specific antibodies that bind to one domain in GREM1 and one or more additional antigens or a bi-specific that binds to two different regions of GREM1 are nonetheless considered antibodies that “specifically bind,” as used herein.

The term “high affinity antibody” refers to those mAbs having a binding affinity to GREM1, expressed as K_{D}, of at least 10⁻⁷ M; preferably 10⁻⁸ M; more preferably 10⁻⁹M, even more preferably 10⁻¹⁰ M, even more preferably 16′¹¹ M, as measured by surface plasmon resonance, e.g., BIACORE™ or solution-affinity ELISA.

By the term “slow off rate,” “Koff,” or “kd” is meant an antibody that dissociates from GREM1, with a rate constant of 1×10⁻³ s⁻¹ or less, preferably 1×10⁻⁴ s⁻¹ or less, as determined by surface plasmon resonance, e.g., BIACORE™

The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. The terms “antigen-binding fragment” of an antibody, or “antibody fragment,” as used herein, refers to one or more fragments of an antibody that retain the ability to bind to GREM1.

In specific embodiments of the methods of the invention, antibody or antibody fragments may be conjugated to a therapeutic moiety (“immunoconjugate”), such as an antibiotic, a second anti-GREM1 antibody, or an antibody to a cytokine such as IL-1, IL-6, or TGF-β, or any other therapeutic moiety for treating PAH.

An “isolated antibody,” as used herein, is intended to refer to an antibody that is substantially free of other antibodies (Abs) having different antigenic specificities, e.g., an isolated antibody that specifically binds human GREM1, or a fragment thereof, is substantially free of Abs that specifically bind antigens other than GREM1.

A “blocking antibody” or a “neutralizing antibody,” as used herein (or an “antibody that neutralizes GREM1 activity”), is intended to refer to an antibody whose binding to GREM1 results in inhibition of at least one biological activity of GREM1. This inhibition of the biological activity of GREM1 can be assessed by measuring one or more indicators of GREM1 biological activity by one or more of several standard in vitro assays (such as a neutralization assay, as described herein) or in vivo assays known in the art (for example, animal models to look at protection from GREM1 activity following administration of one or more of the antibodies described herein).

The term “surface plasmon resonance,” as used herein, refers to an optical phenomenon that allows for the analysis of real-time biomolecular interactions by detection of alterations in protein concentrations within a biosensor matrix, for example using the BIACORE™ system (Pharmacia Biosensor AB, Uppsala, Sweden and Piscataway, N.J.).

The term “K_{D},” as used herein, is intended to refer to the equilibrium dissociation constant of a particular antibody-antigen interaction.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of nonlinear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

The term “substantial identity” or “substantially identical” when referring to a nucleic acid or fragment thereof, indicates that, when optimally aligned with appropriate nucleotide insertions or deletions with another nucleic acid (or its complementary strand), there is nucleotide sequence identity in at least about 90%, and more preferably at least about 95%, 96%, 97%, 98% or 99% of the nucleotide bases, as measured by any well-known algorithm of sequence identity, such as FASTA, BLAST or GAP, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 90% sequence identity, even more preferably at least 95%, 98% or 99% sequence identity. Preferably, residue positions, which are not identical, differ by conservative amino acid substitutions.

A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24: 307-331, which is herein incorporated by reference. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256:1443 45, herein incorporated by reference. A “moderately conservative” replacement is any change having a nonnegative value in the PAM250 log-likelihood matrix.

Sequence similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215: 403-410 and (1997) Nucleic Acids Res. 25: 3389-3402, each of which is herein incorporated by reference.

In specific embodiments, the antibody or antibody fragment for use in the methods of the invention may be mono-specific, bi-specific, or multi-specific. Multi-specific antibodies may be specific for different epitopes of one target polypeptide or may contain antigen-binding domains specific for epitopes of more than one target polypeptide. An exemplary bi-specific antibody format that can be used in the context of the present invention involves the use of a first immunoglobulin (Ig) C_{H}3 domain and a second Ig C_{H}3 domain, wherein the first and second Ig C_{H}3 domains differ from one another by at least one amino acid, and wherein at least one amino acid difference reduces binding of the bi-specific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise an Y96F modification (by IMGT; Y436F by EU). Further modifications that may be found within the second C_{H}3 include: D16E, L18M, N44S, K52N, V57M, and V82I (by IMGT; D356E, L358M, N384S, K392N, V397M, and V422I by EU) in the case of IgG1 mAbs; N44S, K52N, and V82I (IMGT; N384S, K392N, and V422I by EU) in the case of IgG2 mAbs; and Q15R, N44S, K52N, V57M, R69K, E79Q, and V82I (by IMGT; Q355R, N384S, K392N, V397M, R409K, E419Q, and V422I by EU) in the case of IgG4 mAbs. Variations on the bi-specific antibody format described above are contemplated within the scope of the present invention.

It is to be understood that, unless specifically indicated otherwise, the term “antibody,” as used herein, encompasses antibody molecules comprising two immunoglobulin heavy chains and two immunoglobulin light chains (i.e., “full antibody molecules”) as well as antigen-binding fragments thereof. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. The terms “antigen-binding fragment” of an antibody, or “antibody fragment”, as used herein, refers to one or more fragments of an antibody that retain the ability to specifically bind to human GREM1. An antibody fragment may include a Fab fragment, a F(ab′)₂ fragment, a Fv fragment, a dAb fragment, a fragment containing a CDR, or an isolated CDR. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and (optionally) constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}-C_{H}2; (iii) V_{H}-C_{H}3; (iv) V_{H}-CH1-Cₕ2; (V) V_{H}-Cₕ1-Cₕ2-Cₕ3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be mono-specific or multi-specific (e.g., bi-specific). A multi-specific antigen-binding fragment of an antibody will typically comprise at least two different variable domains, wherein each variable domain is capable of specifically binding to a separate antigen or to a different epitope on the same antigen. Any multi-specific antibody format, including the exemplary bi-specific antibody formats disclosed herein, may be adapted for use in the context of an antigen-binding fragment of an antibody of the present invention using routine techniques available in the art.

The anti-human GREM1 antibodies and antibody fragments for use in the present invention encompass proteins having amino acid sequences that vary from those of the described antibodies, but that retain the ability to bind human GREM1. Such variant antibodies and antibody fragments comprise one or more additions, deletions, or substitutions of amino acids when compared to parent sequence, but exhibit biological activity that is essentially equivalent to that of the described antibodies. Likewise, the antibody-encoding DNA sequences of the present invention encompass sequences that comprise one or more additions, deletions, or substitutions of nucleotides when compared to the disclosed sequence, but that encode an antibody or antibody fragment that is essentially bioequivalent to an antibody or antibody fragment of the invention.

Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single dose or multiple doses. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, and potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and/or in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of the antibodies of the invention may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antibodies may include antibody variants comprising amino acid changes, which modify the glycosylation characteristics of the antibodies, e.g., mutations that eliminate or remove glycosylation.

According to certain embodiments of the present invention, anti-GREM1 antibodies for use in the methods of the present invention comprise an Fc domain comprising one or more mutations that enhance or diminish antibody binding to the FcRn receptor, e.g., at acidic pH as compared to neutral pH. For example, the present invention includes anti-GREM1 antibodies comprising a mutation in the C_{H}2 or a C_{H}3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., H/L/R/S/P/Q or K) and/or 434 (e.g., A, W, H, F or Y [N434A, N434W, N434H, N434F or N434Y]); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 259I (e.g., V259I), and 308F {e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P). In yet another embodiment, the modification comprises a 265A (e.g., D265A) and/or a 297A (e.g., N297A) modification.

For example, the present invention includes anti-GREM1 antibodies comprising an Fc domain comprising one or more pairs or groups of mutations selected from the group consisting of: 250Q and 248L (e.g., T250Q and M248L); 252Y, 254T and 256E (e.g., M252Y, S254T and T256E); 428L and 434S (e.g., M428L and N434S); 257I and 31 11 (e.g., P257I and Q31 11); 257I and 434H (e.g., P257I and N434H); 376V and 434H (e.g., D376V and N434H); 307A, 380A and 434A (e.g., T307A, E380A and N434A); and 433K and 434F (e.g., H433K and N434F). All possible combinations of the foregoing Fc domain mutations, and other mutations within the antibody variable domains disclosed herein, are contemplated within the scope of the present invention.

The present invention also includes anti-GREM1 antibodies comprising a chimeric heavy chain constant (C_{H}) region, wherein the chimeric C_{H} region comprises segments derived from the CH regions of more than one immunoglobulin isotype. For example, the antibodies of the invention may comprise a chimeric C_{H} region comprising part or all of a C_{H}2 domain derived from a human IgG1, human IgG2 or human IgG4 molecule, combined with part or all of a C_{H}3 domain derived from a human IgG1, human IgG2 or human IgG4 molecule. According to certain embodiments, the antibodies of the invention comprise a chimeric C_{H} region having a chimeric hinge region. For example, a chimeric hinge may comprise an “upper hinge” amino acid sequence (amino acid residues from positions 216 to 227 according to EU numbering) derived from a human IgG1, a human IgG2 or a human IgG4 hinge region, combined with a “lower hinge” sequence (amino acid residues from positions 228 to 236 according to EU numbering) derived from a human IgG1, a human IgG2 or a human IgG4 hinge region.

According to certain embodiments, the chimeric hinge region comprises amino acid residues derived from a human IgG1 or a human IgG4 upper hinge and amino acid residues derived from a human IgG2 lower hinge. An antibody comprising a chimeric C_{H} region as described herein may, in certain embodiments, exhibit modified Fc effector functions without adversely affecting the therapeutic or pharmacokinetic properties of the antibody. (See, e.g., U.S. Provisional Appl. No. 61/759,578, filed Feb. 1, 2013, the disclosure of which is hereby incorporated by reference in its entirety).

In general, the antibodies for use in the methods of the present invention may function by binding to human GREM1. In some embodiments, the antibodies of the present invention may bind to the catalytic domain of human GREM1, or to a fragment thereof. In some embodiments, the antibodies of the invention may bind to the secreted form of human GREM1 or to the membrane-associated form of human GREM1. In some embodiments, the antibodies of the present invention may bind to more than one domain (cross-reactive antibodies).

In certain embodiments of the invention, the antibodies may bind to an epitope located in the region between amino acid residues 25-184 of SEQ ID NO: 594 or SEQ ID NO: 595.

In certain embodiments, the antibodies for use in the methods of the present invention may function by blocking or inhibiting BMP signaling by binding to any other region or fragment of the full length native protein, the amino acid sequence of which is shown in SEQ ID NO: 594, which is encoded by the nucleic acid sequence shown in SEQ ID NO: 593. In one embodiment, the antibodies of the present invention may function by reversing the inhibition of BMP2, BMP4 or BMP7 by binding to full-length GREM1 or a fragment thereof. In some embodiments, the antibodies of the present invention may function by promoting BMP signaling or may block the binding between GREM1 and BMPs including BMP2, BMP4 or BMP7.

In certain embodiments, the antibodies for use in the methods of the present invention may function by blocking GREM1 binding to heparin and/or by inhibiting heparin-mediated VEGFR-2 activation.

In certain embodiments, the antibodies for use in the methods of the present invention may be bi-specific antibodies. The bi-specific antibodies of the invention may bind one epitope in one domain and may also bind one epitope in a second domain of human GREM1. In certain embodiments, the bi-specific antibodies of the invention may bind two different epitopes in the same domain.

In one embodiment, a fully human monoclonal antibody or antigen-binding fragment thereof that binds to human GREM1 may be used in the methods of the invention, wherein the antibody or fragment thereof exhibits one or more of the following characteristics: (i) comprises a HCVR having an amino acid sequence selected from the group consisting of SEQ ID NO: 2, 18, 34, 50, 66, 82, 98, 1 14, 130, 146, 162, 178, 194, 210, 226, 242, 258, 274, 290, 306, 322, 338, 354, 370, 386, 402, 418, 434, 450, 466, 482, 498, 514, 530, 546, 562, and 578, or a substantially similar sequence thereof having at least 90%, at least 95%, at least 98% or at least 99% sequence identity; (ii) comprises a LCVR having an amino acid sequence selected from the group consisting of SEQ ID NO: 10, 26, 42, 58, 74, 90, 106, 122, 138, 154, 170, 186, 202, 218, 234, 250, 266, 282, 298, 314, 330, 346, 362, 378, 394, 410, 426, 442, 458, 474, 490, 506, 522, 538, 554, 570, and 586, or a substantially similar sequence thereof having at least 90%, at least 95%, at least 98% or at least 99% sequence identity; (iii) comprises a HCDR3 domain having an amino acid sequence selected from the group consisting of SEQ ID NO: 8, 24, 40, 56, 72, 88, 104, 120, 136, 152, 168, 184, 200, 216, 232, 248, 264, 280, 296, 312, 328, 344, 360, 376, 392, 408, 424, 440, 456, 472, 488, 504, 520, 536, 552, 568, and 584, or a substantially similar sequence thereof having at least 90%, at least 95%, at least 98% or at least 99% sequence identity; and a LCDR3 domain having an amino acid sequence selected from the group consisting of SEQ ID NO: 16, 32, 48, 64, 80, 96, 1 12, 128, 144, 160, 176, 192, 208, 224, 240, 256, 272, 288, 304, 320, 336, 352, 368, 384, 400, 416, 432, 448, 464, 480, 496, 512, 528, 544, 560, 576, and 592, or a substantially similar sequence thereof having at least 90%, at least 95%, at least 98% or at least 99% sequence identity; (iv) comprises a HCDR1 domain having an amino acid sequence selected from the group consisting of SEQ ID NO: 4, 20, 36, 52, 68, 84, 100, 1 16, 132, 148, 164, 180, 196, 212, 228, 244, 260, 276, 292, 308, 324, 340, 356, 372, 388, 404, 420, 436, 452, 468, 484, 500, 516, 532, 548, 564, and 580, or a substantially similar sequence thereof having at least 90%, at least 95%, at least 98% or at least 99% sequence identity; a HCDR2 domain having an amino acid sequence selected from the group consisting of SEQ ID NO: 6, 22, 38, 54, 70, 86, 102, 1 18, 134, 150, 166, 182, 198, 214, 230, 246, 262, 278, 294, 310, 326, 342, 358, 374, 390, 406, 422, 438, 454, 470, 486, 502, 518, 534, 550, 566, and 582, or a substantially similar sequence thereof having at least 90%, at least 95%, at least 98% or at least 99% sequence identity; a LCDR1 domain having an amino acid sequence selected from the group consisting of SEQ ID NO: 12, 28, 44, 60, 76, 92, 108, 124, 140, 156, 172, 188, 204, 220, 236, 252, 268, 284, 300, 316, 332, 348, 364, 380, 396, 412, 428, 444, 460, 476, 492, 508, 524, 540, 556, 572, and 588, or a substantially similar sequence thereof having at least 90%, at least 95%, at least 98% or at least 99% sequence identity; and a LCDR2 domain having an amino acid sequence selected from the group consisting of SEQ ID NO: 14, 30, 46, 62, 78, 94, 1 10, 126, 142, 158, 174, 190, 206, 222, 238, 254, 270, 286, 302, 318, 334, 350, 366, 382, 398, 414, 430, 446, 462, 478, 494, 510, 526, 542, 558, 574, and 590, or a substantially similar sequence thereof having at least 90%, at least 95%, at least 98% or at least 99% sequence identity; (v) binds to GREM1 with a K_{D} equal to or less than 10⁻⁷; (vi) blocks GREM1 binding to one of BMP2, BMP4 or BMP7; (vii) blocks GREM1 inhibition of BMP signaling and promotes cell differentiation; and (viii) blocks GREM1 binding to heparin.

Certain anti-GREM1 antibodies for use in the methods of the present invention are able to bind to and neutralize the activity of GREM1, as determined by in vitro or in vivo assays. The ability of the antibodies of the invention to bind to and neutralize the activity of GREM1 may be measured using any standard method known to those skilled in the art, including binding assays, or activity assays, as described herein.

Non-limiting, exemplary in vitro assays for measuring binding activity include surface plasmon resonance conducted on, e.g., a T200 Biacore instrument. Blocking assays may be used to determine the ability of the anti-GREM1 antibodies to block the BMP4 binding ability of GREM1 in vitro. The activity of the anti-GREM1 antibodies in promoting BMP4 signaling and cell differentiation of osteoblast progenitor cells in response to BMP4 signaling may be assessed as may the inhibition of the GREM1-heparin binding interaction using the anti-GREM1 antibodies described herein.

The present invention also includes anti-GREM1 antibodies and antigen binding fragments thereof which bind to at least one biologically active fragment of any of the following proteins, or peptides: SEQ ID NO: 594 (full length native human GREM1), or SEQ ID NO: 595 (recombinant form of human GREM1) for use in the methods of the invention. Any of the GREM1 peptides described herein, or fragments thereof, may be used to generate anti-GREM1 antibodies.

The peptides may be modified to include addition or substitution of certain residues for tagging or for purposes of conjugation to carrier molecules, such as, KLH. For example, a cysteine may be added at either the N terminal or C terminal end of a peptide, or a linker sequence may be added to prepare the peptide for conjugation to, for example, KLH for immunization.

The antibodies specific for GREM1 may contain no additional labels or moieties, or they may contain an N-terminal or C-terminal label or moiety. In one embodiment, the label or moiety is biotin. In a binding assay, the location of a label (if any) may determine the orientation of the peptide relative to the surface upon which the peptide is bound. For example, if a surface is coated with avidin, a peptide containing an N-terminal biotin will be oriented such that the C-terminal portion of the peptide will be distal to the surface. In one embodiment, the label may be a radionuclide, a fluorescent dye or a MRI-detectable label. In certain embodiments, such labeled antibodies may be used in diagnostic assays including imaging assays.

The present invention includes the use of anti-GREM1 antibodies which interact with one or more amino acids found within one or more regions of GREM1. The epitope to which the antibodies bind may consist of a single contiguous sequence of 3 or more (e.g., 3, 4, 5, 6, 7, 8, 9, 10, 11, 12, 13, 14, 15, 16, 17, 18, 19, 20 or more) amino acids located within any of the aforementioned regions of the GREM1 molecule (e.g. a linear epitope in a domain). Alternatively, the epitope may consist of a plurality of non-contiguous amino acids (or amino acid sequences) located within either or both of the aforementioned regions of the GREM1 molecule (e.g. a conformational epitope).

Various techniques known to persons of ordinary skill in the art can be used to determine whether an antibody “interacts with one or more amino acids” within a polypeptide or protein. Exemplary techniques include, for example, routine cross-blocking assays, such as that described in Antibodies, Harlow and Lane (Cold Spring Harbor Press, Cold Spring Harbor, N.Y.). Other methods include alanine scanning mutational analysis, peptide blot analysis (Reineke (2004) Methods Mol Biol 248:443-63), peptide cleavage analysis crystallographic studies and NMR analysis. In addition, methods such as epitope excision, epitope extraction and chemical modification of antigens can be employed (Tomer (2000) Protein Science 9:487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues that correspond to the specific amino acids with which the antibody interacts. See, e.g., Ehring (1999) Analytical Biochemistry 267(2):252-259; Engen and Smith (2001) Anal. Chem. 73: 256A-265A.

The term “epitope” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

Modification-Assisted Profiling (MAP), also known as Antigen Structure-based Antibody Profiling (ASAP) is a method that categorizes large numbers of monoclonal antibodies (mAbs) directed against the same antigen according to the similarities of the binding profile of each antibody to chemically or enzymatically modified antigen surfaces (see, e.g., U.S. Patent Publication No. 2004/0101920, herein specifically incorporated by reference in its entirety). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP may facilitate identification of rare hybridoma clones that produce mAbs having the desired characteristics. MAP may be used to sort the antibodies of the invention into groups of antibodies binding different epitopes.

In certain embodiments, the anti-GREM1 antibodies or antigen-binding fragments thereof for use in the methods of the invention bind an epitope within any one or more of the regions exemplified in GREM1, either in natural form, as exemplified in SEQ ID NO: 594, or recombinantly produced, as exemplified in SEQ ID NO: 595, or to a fragment thereof. In certain embodiments, the antibodies for use in the methods of the invention, as shown in Table 1, interact with at least one amino acid sequence selected from the group consisting of amino acid residues ranging from about position 1 to about position 24 of SEQ ID NO: 594; or amino acid residues ranging from about position 25 to about position 184 of SEQ ID NO: 594. These regions are further exemplified in SEQ ID NO: 595.

The present invention includes the use of anti-human GREM1 antibodies that bind to the same epitope, or a portion of the epitope, as any of the specific exemplary antibodies described herein in Table 1, or an antibody having the CDR sequences of any of the exemplary antibodies described in Table 1. Likewise, the present invention also includes anti-human GREM1 antibodies that compete for binding to GREM1 or a GREM1 fragment with any of the specific exemplary antibodies described herein in Table 1, or an antibody having the CDR sequences of any of the exemplary antibodies described in Table 1.

One can easily determine whether an antibody binds to the same epitope as, or competes for binding with, a reference anti-GREM1 antibody by using routine methods known in the art. For example, to determine if a test antibody binds to the same epitope as a reference anti-GREM1 antibody of the invention, the reference antibody is allowed to bind to a GREM1 protein or peptide under saturating conditions. Next, the ability of a test antibody to bind to the GREM1 molecule is assessed. If the test antibody is able to bind to GREM1 following saturation binding with the reference anti-GREM1 antibody, it can be concluded that the test antibody binds to a different epitope than the reference anti-GREM1 antibody. On the other hand, if the test antibody is not able to bind to the GREM1 protein following saturation binding with the reference anti-GREM1 antibody, then the test antibody may bind to the same epitope as the epitope bound by the reference anti-GREM1 antibody of the invention.

To determine if an antibody competes for binding with a reference anti-GREM1 antibody, the above-described binding methodology is performed in two orientations: In a first orientation, the reference antibody is allowed to bind to a GREM1 protein under saturating conditions followed by assessment of binding of the test antibody to the GREM1 molecule. In a second orientation, the test antibody is allowed to bind to a GREM1 molecule under saturating conditions followed by assessment of binding of the reference antibody to the GREM1 molecule. If, in both orientations, only the first (saturating) antibody is capable of binding to the GREM1 molecule, then it is concluded that the test antibody and the reference antibody compete for binding to GREM1. As will be appreciated by a person of ordinary skill in the art, an antibody that competes for binding with a reference antibody may not necessarily bind to the identical epitope as the reference antibody, but may sterically block binding of the reference antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. 1990 50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art.

The invention encompasses use of a human anti-GREM1 monoclonal antibody conjugated to a therapeutic moiety (“immunoconjugate””). As used herein, the term “immunoconjugate” refers to an antibody that is chemically or biologically linked to a radioactive agent, a cytokine, an interferon, a target or reporter moiety, an enzyme, a toxin, or a therapeutic agent. The antibody may be linked to the radioactive agent, cytokine, interferon, target or reporter moiety, enzyme, toxin, or therapeutic agent at any location along the molecule so long as it is able to bind its target. An example of immunoconjugate is antibody drug conjugate. In some embodiments, the agent may be a second different antibody to human GREM1, or to a cytokine such as IL-1, IL-6, or a chemokine such as TGF-β. The type of therapeutic moiety that may be conjugated to the anti-GREM1 antibody and will take into account the condition to be treated and the desired therapeutic effect to be achieved. Examples of suitable agents for forming immunoconjugates are known in the art; see for example, WO 05/103081. The preparation of immunoconjugates and immunotoxins is generally well known in the art (see, e.g., U.S. Pat. No. 4,340,535). Immunoconjugates are described in detail, for example, in U.S. Pat. Nos. 7,250,492, 7,420,040 and 7,411,046, each of which is incorporated herein in their entirety.

The antibodies for use in the methods of the present invention may be mono-specific, bi-specific, or multi-specific. Multi-specific antibodies may be specific for different epitopes of one target polypeptide or may contain antigen-binding domains specific for more than one target polypeptide. See, e.g., Tutt et al., 1991, J. Immunol. 147:60-69; Kufer et al., 2004, Trends Biotechnol. 22:238-244. The antibodies of the present invention can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bi-specific or a multi-specific antibody with a second binding specificity. For example, the present invention includes bi-specific antibodies wherein one arm of an immunoglobulin is specific for the N-terminal region of GREM1, or a fragment thereof, and the other arm of the immunoglobulin is specific for the C-terminal region of GREM1, or a second therapeutic target, or is conjugated to a therapeutic moiety. An exemplary bi-specific antibody format that can be used in the context of the present invention involves the use of a first immunoglobulin (Ig) C_{H}3 domain and a second Ig C_{H}3 domain, wherein the first and second Ig C_{H}3 domains differ from one another by at least one amino acid, and wherein at least one amino acid difference reduces binding of the bi-specific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). Further modifications that may be found within the second C_{H}3 include: D16E, L18M, N44S, K52N, V57M, and V82I (by IMGT; D356E, L358M, N384S, K392N, V397M, and V422I by EU) in the case of IgG1 antibodies; N44S, K52N, and V82I (IMGT; N384S, K392N, and V422I by EU) in the case of IgG2 antibodies; and Q15R, N44S, K52N, V57M, R69K, E79Q, and V82I (by IMGT; Q355R, N384S, K392N, V397M, R409K, E419Q, and V422I by EU) in the case of IgG4 antibodies. Variations on the bi-specific antibody format described above are contemplated within the scope of the present invention.

Other exemplary bispecific formats that can be used in the context of the present invention include, without limitation, e.g., scFv-based or diabody bispecific formats, IgG-scFv fusions, dual variable domain (DVD)-1g, Quadroma, knobs-into-holes, common light chain (e.g., common light chain with knobs-into-holes, etc.), CrossMab, CrossFab, (SEED)body, leucine zipper, Duobody, IgG1/IgG2, dual acting Fab (DAF)-IgG, and Mab² bispecific formats (see, e.g., Klein et al. 2012, mAbs 4:6, 1-1 1, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J. Am. Chem. Soc. [Epub: Dec. 4, 2012]).

Methods for generating monoclonal antibodies, including fully human monoclonal anti-GREM1 antibodies, or antigen-binding fragments thereof, suitable for use in the methods of the present invention are known in the art. Any such known methods can be used in the context of the present invention to make human antibodies that specifically bind to human GREM1.

In certain embodiments, the antibodies, or antigen-binding fragments thereof, for use in the present invention are obtained from mice immunized with a primary immunogen, such as a native, full length human GREM1 (See, e.g., GenBank accession number NP_037504 (SEQ ID NO: 594)) or with a recombinant form of GREM1 (SEQ ID NO: 595) or GREM1 fragments, followed by immunization with a secondary immunogen, or with an immunogenically active fragment of GREM1.

The immunogen may be an immunogenic fragment of human GREM1 or DNA encoding the fragment thereof. The immunogen may GREM1 coupled to a histidine tag and/or to a fragment of Fc region of an antibody.

The amino acid sequence of full length human GREM1 (also known by Gen bank accession number NP-037504) is shown as SEQ ID NO: 594. The full-length amino acid sequence of recombinant GREM1 (amino acid residues 25-184 GREM1 coupled to Fc region and a histidine tag) is shown as SEQ ID NO: 595.

The full-length DNA sequence of GREM1 is shown as SEQ ID NO: 593.

In certain embodiments, antibodies that bind specifically to human GREM1 may be prepared using fragments of the above-noted regions, or peptides that extend beyond the designated regions by about 5 to about 20 amino acid residues from either, or both, the N or C terminal ends of the regions described herein. In certain embodiments, any combination of the above-noted regions or fragments thereof may be used in the preparation of human GREM1 specific antibodies. In certain embodiments, any one or more of the above-noted regions of human GREM1, or fragments thereof may be used for preparing monospecific, bispecific, or multispecific antibodies.

Methods for generating human antibodies in transgenic mice are also known in the art. Any such known methods can be used in the context of the present invention to make human antibodies that specifically bind to human GREM1.

Using VELOCIMMUNE™ technology (see, for example, U.S. Pat. No. 6,596,541, Regeneron Pharmaceuticals, VELOCIMMUNE®) or any other known method for generating monoclonal antibodies, high affinity chimeric antibodies to human GREM1 are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. As in the experimental section below, the antibodies are characterized and selected for desirable characteristics, including affinity, selectivity, epitope, etc. The mouse constant regions are replaced with a desired human constant region to generate the fully human antibody of the invention, for example wild-type or modified IgG1 or IgG4. While the constant region selected may vary according to specific use, high affinity antigen-binding and target specificity characteristics reside in the variable region.

In general, anti-GREM1 antibodies for use in the methods of the instant invention possess very high affinities, typically possessing K_{D} of from about 10⁻¹² through about 10⁻⁷ M, when measured by binding to antigen either immobilized on solid phase or in solution phase. While the constant region of the antibodies may vary according to specific use, high affinity antigen-binding and target specificity characteristics reside in the variable region.

An anti-GREM1 antibody, or antigen-binding fragment thereof, for use in the methods of the present invention may be present in a pharmaceutical composition. Such pharmaceutical compositions are formulated with suitable carriers, excipients, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™, Life Technologies, Carlsbad, Calif.), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA, J Pharm Sci Technol 52:238-311 (1998).

Various delivery systems are known and can be used to administer a pharmaceutical composition comprising an anti-GREM1 antibody, or antigen-binding fragment thereof, e.g., encapsulation in liposomes, microparticles, microcapsules, recombinant cells capable of expressing the antibody, receptor mediated endocytosis (see, e.g., Wu et al., J Biol Chem 262:4429-4432 (1987)). The antibodies may also be delivered by gene therapy techniques. Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

A pharmaceutical composition comprising an anti-GREM1 antibody, or antigen-binding fragment thereof, can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present invention include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L.P.), and the HUMIRA™ Pen (Abbott Labs, Abbott Park Ill.), to name only a few.

In certain situations, the pharmaceutical composition can be delivered in a controlled release system. In one embodiment, a pump may be used (see Langer, supra; Sefton, CRC Crit. Ref Biomed. Eng. 14:201 (1987)). In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, Science 249:1527-1533 (1990).

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to about 500 mg per dosage form in a unit dose; especially in the form of injection, it is preferred that the aforesaid antibody is contained in about 5 to about 100 mg and in about 10 to about 250 mg for the other dosage forms.

This invention is further illustrated by the following examples which should not be construed as limiting. The entire contents of all references, patents and published patent applications cited throughout this application, as well as the Figures and the Sequence Listing, are hereby incorporated herein by reference.

### Examples

### Example 1. Gremlin 1 Binding Proteins

U.S. Patent Publication No. 2016/0024195, the entire contents of which are incorporated herein by reference, describes the generation and characterization of chimeric and fully human anti-GREM1 antibodies (i.e., antibodies possessing human variable domains and human constant domains) suitable for use in the present invention. For example, several anti-GREM1 antibodies including as cross-reactive and chimeric antibodies (i.e., antibodies possessing human variable domains and mouse constant domains) were obtained, and include those antibodies designated as H1M2907N, H2M2780N, H2M2782N, H2M2783N, H4H2783N2, H2M2784N, H2M2785N, H2M2786N, H2M2889N, H2M2890N, H2M2891N, H2M2892N, H2M2895N, H2M2897N, H2M2898N, H2M2899N, H2M2901N, H2M2906N, H2M2926N, H3M2788N, and H3M2929N.

Additional fully human anti-GREM1 antibodies were also obtained and include those antibodies designated as follows: H4H6232P, H4H6233P, H4H6236P, H4H6238P, H4H6240P, H4H6243P, H4H6245P, H4H6246P, H4H6248P, H4H6250P, H4H6251P, H4H6252S, H4H6256P, H4H6260P, H4H6269P, and H4H6270P.

Table 1 sets forth the heavy and light chain variable region amino acid sequence pairs of selected antibodies specific for human GREM1 and their corresponding antibody identifiers suitable for use in the methods of the present invention. Antibodies are typically referred to herein according to the following nomenclature: Fc prefix (e.g. “H4H”, “H1M, “H2M”), followed by a numerical identifier (e.g. “2907” as shown in Table 1), followed by a “P” or “N” suffix. Thus, according to this nomenclature, an antibody may be referred to as, e.g. “H1H2907”. The H4H, H1M, and H2M prefixes on the antibody designations used herein indicate the particular Fc region of the antibody. For example, an “H2M” antibody has a mouse IgG2 Fc, whereas an “H4H” antibody has a human IgG4 Fc. As will be appreciated by a person of ordinary skill in the art, an H1M or H2M antibody can be converted to an H4H antibody, and vice versa, but in any event, the variable domains (including the CDRs), which are indicated by the numerical identifiers shown in Table 1, will remain the same. Antibodies having the same numerical antibody designation, but differing by a letter suffix of N, B or P refer to antibodies having heavy and light chains with identical CDR sequences but with sequence variations in regions that fall outside of the CDR sequences (i.e., in the framework regions). Thus, N, B and P variants of a particular antibody have identical CDR sequences within their heavy and light chain variable regions but differ from one another within their framework regions.

TABLE 1

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| 2907N | 2 | 4 | 6 | 8 | 10 | 12 | 14 | 16 |
| 2780N | 18 | 20 | 22 | 24 | 26 | 28 | 30 | 32 |
| 2782N | 34 | 36 | 38 | 40 | 42 | 44 | 46 | 48 |
| 2783N | 50 | 52 | 54 | 56 | 58 | 60 | 62 | 64 |
| 2783N2 | 66 | 68 | 70 | 72 | 74 | 76 | 78 | 80 |
| 2784N | 82 | 84 | 86 | 88 | 90 | 92 | 94 | 96 |
| 2785N | 98 | 100 | 102 | 104 | 106 | 108 | 110 | 112 |
| 2786N | 114 | 116 | 118 | 120 | 122 | 124 | 126 | 128 |
| 2889N | 130 | 132 | 134 | 136 | 138 | 140 | 142 | 144 |
| 2890N | 146 | 148 | 150 | 152 | 154 | 156 | 158 | 160 |
| 2891N | 162 | 164 | 166 | 168 | 170 | 172 | 174 | 176 |
| 2892N | 178 | 180 | 182 | 184 | 186 | 188 | 190 | 192 |
| 2895N | 194 | 196 | 198 | 200 | 202 | 204 | 206 | 208 |
| 2897N | 210 | 212 | 214 | 216 | 218 | 220 | 222 | 224 |
| 2898N | 226 | 228 | 230 | 232 | 234 | 236 | 238 | 240 |
| 2899N | 242 | 244 | 246 | 248 | 250 | 252 | 254 | 256 |
| 2901N | 258 | 260 | 262 | 264 | 266 | 268 | 270 | 272 |
| 2906N | 274 | 276 | 278 | 280 | 282 | 284 | 286 | 288 |
| 2926N | 290 | 292 | 294 | 296 | 298 | 300 | 302 | 304 |
| 2788N | 306 | 308 | 310 | 312 | 314 | 316 | 318 | 320 |
| 2929N | 322 | 324 | 326 | 328 | 330 | 332 | 334 | 336 |
| 6232P | 338 | 340 | 342 | 344 | 346 | 348 | 350 | 352 |
| 6233P | 354 | 356 | 358 | 360 | 362 | 364 | 366 | 368 |
| 6236P | 370 | 372 | 374 | 376 | 378 | 380 | 382 | 384 |
| 6238P | 386 | 388 | 390 | 392 | 394 | 396 | 398 | 400 |
| 6240P | 402 | 404 | 406 | 408 | 410 | 412 | 414 | 416 |
| 6243P | 418 | 420 | 422 | 424 | 426 | 428 | 430 | 432 |
| 6245P | 434 | 436 | 438 | 440 | 442 | 444 | 446 | 448 |
| 6246P | 450 | 452 | 454 | 456 | 458 | 460 | 462 | 464 |
| 6248P | 466 | 468 | 470 | 472 | 474 | 476 | 478 | 480 |
| 6250P | 482 | 484 | 486 | 488 | 490 | 492 | 494 | 496 |
| 6251P | 498 | 500 | 502 | 504 | 506 | 508 | 510 | 512 |
| 6252P | 514 | 516 | 518 | 520 | 522 | 524 | 526 | 528 |
| 6256P | 530 | 532 | 534 | 536 | 538 | 540 | 542 | 544 |
| 6260P | 546 | 548 | 550 | 552 | 554 | 556 | 558 | 560 |
| 6269P | 562 | 564 | 566 | 568 | 570 | 572 | 574 | 576 |
| 6270P | 578 | 580 | 582 | 584 | 586 | 588 | 590 | 592 |

### Example 2. Anti-Gremlin-1 Antibody Treatment Restores Pulmonary Artery Diameter and Restores Right Ventricular Cardiac Function in a Mouse Model of Chronic Hypoxia

To evaluate the effect of the anti-gremlin-1 antibody, H4H6245P2, in pulmonary arterial hypertension, two separate studies using a chronic hypoxia-induced pulmonary arterial hypertension mouse model were performed.

The following materials and methods were used for these studies.

### Materials and Methods

Mice

For both studies, eleven to thirteen-week-old Taconic C57BL/6 mice were used. Mice were separated into treatment groups by weight such that starting body weights were similar among different groups. Cages were selected to either remain at about 21% O₂ (normobaric normoxia) or placed into a 10% O₂ (normobaric hypoxia) chamber (a modified 3′ Semi-Rigid Isolator unit, Charles River) that maintained low O₂ levels with adjustment of N₂ flow to a steady intake of room air.

For the first study (Study 1), mice were administered drugs or saline starting on day 14. A group of mice (n=10) housed in normobaric normoxia cages were subcutaneously administered saline at 5 mL/kg twice per week for two weeks, while mice housed in normobaric hypoxia cages were separated into 3 treatment groups including a group of mice (n=10) subcutaneously treated with saline at 5 mL/kg twice per week for two weeks, a group of mice (n=10) subcutaneously administered an isotype control antibody at 25 mg/kg twice per week for two weeks, and a group of mice subcutaneously treated with an anti-Gremlin-1 antibody, H4H6245P2, (n=10) at 25 mg/kg twice a week for two weeks.

For the second study (Study 2), mice were administered drugs or saline starting on day 14. A group of mice (n=10) housed in normobaric normoxia cages were subcutaneously administered saline at 5 mL/kg twice per week for four weeks, while mice housed in normobaric hypoxia cages were separated into 5 treatment groups including a group of mice (n=10) subcutaneously treated with saline at 5 mL/kg twice per week for four weeks, a group of mice (n=10) subcutaneously administered an isotype control antibody at 25 mg/kg twice per week for four weeks, a group of mice (n=10) subcutaneously treated with anti-Gremlin-1 antibody, H4H6245P2, at 10 mg/kg twice a week for four weeks, a group of mice (n=9) subcutaneously treated with anti-Gremlin-1 antibody, H4H6245P2, at 25 mg/kg twice a week for four weeks, and a group of mice (n=10) subcutaneously treated with anti-Gremlin-1 antibody, H4H6245P2, at 40 mg/kg twice a week for four weeks.

The dosing schedules for Study 1 and Study 2 are provided in Table 2.

TABLE 2
Therapeutic dosing and treatment protocol for each group in chronic hypoxia
mouse model studies
Study 1: 4 week chronic hypoxia with drug dosing beginning after 14 days in hypoxia

| | | | | | | Number of |
|---|---|---|---|---|---|---|
| | | | | | | mice/group |
| Group | Condition | Treatment | Dosage | Frequency | Route | “n” size |
| 1 | Normobaric | Saline | 5 mL/kg | 2x/wk | SC | 10 |
| | normoxia | | | | | |
| 2 | Normobaric | Saline | 5 mL/kg | 2x/wk | SC | 10 |
| | hypoxia | | | | | |
| 3 | Normobaric | Isotype control | 25 mg/kg | 2x/wk | SC | 10 |
| | hypoxia | antibody | | | | |
| 4 | Normobaric | Anti-Gremlin-1 | 25 mg/kg | 2x/wk | SC | 10 |
| | hypoxia | antibody | | | | |

Study 2: 6 week chronic hypoxia with drug dosing beginning after 14 days in hypoxia

| Group | Condition | Treatment | Dosage | Frequency | Route | “n” size |
|---|---|---|---|---|---|---|
| 1 | Normobaric | Saline | 5 mL/kg | 2x/wk | SC | 10 |
| | normoxia | | | | | |
| 2 | Normobaric | Saline | 5 mL/kg | 2x/wk | SC | 10 |
| | hypoxia | | | | | |
| 3 | Normobaric | Isotype control | 25 mg/kg | 2x/wk | SC | 10 |
| | hypoxia | antibody | | | | |
| 4 | Normobaric | Anti-Gremlin-1 | 10 mg/kg | 2x/wk | SC | 10 |
| | hypoxia | antibody | | | | |
| 5 | Normobaric | Anti-Gremlin-1 | 25 mg/kg | 2x/wk | SC | 9 |
| | hypoxia | antibody | | | | |
| 6 | Normobaric | Anti-Gremlin-1 | 40 mg/kg | 2x/wk | SC | 10 |
| | hypoxia | antibody | | | | |

SC = subcutaneous

Ultrasound Assessment and Analysis

On the last day of each study, pulmonary artery size and right ventricular function and dimensions were assessed in each mouse using a high frequency ultrasound system (Vevo 2100, VisualSonics). For the assessment, mice were anesthetized (with 1.5% isoflurane at a rate of 1.0 cc/mL of medical grade air) and their temperature was monitored with a rectal temperature probe and held at approximately 37° C. with a heated platform (MouseMonitorS, Indus Instruments) and a warming lamp. Both brightness-mode (B-mode) and motion-mode (M-mode) imaging were used. B-mode imaging of the mouse heart in cross-section was used to determine pulmonary artery cross-sectional area (PA CSA) at the level of the pulmonary valve. M-mode imaging was used to determine the pulsed wave velocity time integral (VTI), which is derived from the area under the curve of representative Doppler tracings of blood flow through the pulmonary artery. Right ventricular stroke volume (RV SV) was calculated from the product of PA CSA and VTI. Right ventricular cardiac output (RV CO) was calculated from the product of SV and heart rate (HR). M-mode imaging was used to determine right ventricular free wall (RVFW) thickness during diastole and systole. Animals were returned to their home cages before right ventricular pressure assessment.

Right Ventricular Pressure Assessment

Right ventricular pressure was subsequently assessed for all treatment groups. Mice were anesthetized with isoflurane and were kept at approximately 37° C. using a heated platform (Heated Hard Pad 1, Braintree Scientific) and circulating heated water pump (T/Pump Classic, Gaymar Industries). The neck area for each mouse was prepared for surgery by depilating over the right common carotid artery and right jugular vein. An incision was made and the right jugular vein was isolated with care as to not damage the carotid artery and/or the vagus nerve. A piece of 5-0 silk suture was placed under the isolated jugular vein to allow for retraction of the vessel cranially, then a 30-gauge needle was used to introduce a hole into the jugular vein. A pressure catheter (Micro-tip catheter transducer SPR-1000, Millar Instruments, Inc.) was inserted into the opening of the jugular vein and advanced past the right atrium into the right ventricle. The catheter was connected to pressure/volume instrument (MPVS-300, Millar Instruments, Inc.) that measured heart rate as well as both diastolic and systolic right ventricular pressures. These parameters were digitally acquired using a data acquisition system (PowerLab 4/35, ADInstruments). LabChart Pro 7.0 software (ADInstruments) was used to analyze right ventricular pressures. Readings were quantified from a 60 second interval of the pressure tracing (following a 2 minute period of recording to allow for pressure stabilization). The parameters analyzed were right ventricular systolic pressures (RVSP), heart rate (HR) and rate of right ventricular pressure rise (dP/dt max).

Serum/Tissue Collection and Assessment of Right Ventricular Hypertrophy

Following completion of right ventricular pressure measurement, the catheter was removed and each animal was sacrificed. The abdomen was opened and blood was drawn from the Vena Cava for hematocrit assessment and serum collection. The thoracic cavity was then opened and the middle lobe of the right lung was ligated with 5-0 silk suture, excised, placed in RNA later (Sigma-Aldrich, cat #R0901) and frozen 24 hours later at −80° C. The heart was excised from each animal, and the right ventricle (RV) was carefully cut away from the left ventricle and septum (LV+S). Both pieces of heart tissue were separately weighed on a microbalance (AJ000, Mettler) to calculate the index of RV hypertrophy [RV/(LV+S); Fulton Index].

Half of the animals from each treatment group had the lungs perfused at 20-25 mmHg with phosphate buffered solution (PBS, pH 7.4), then fixed with 10% neutral-buffered formalin (NBF). Lungs remained in 10% NBF for 24 hours before being placed into 70% ethanol for at least 48 hours, before tissue processing and paraffin embedding. For animals that did not undergo perfusion-fixation of the lung, the right inferior lobe was ligated with 5-0 silk suture before being excised, weighed and frozen in liquid N₂.

### Results

### Gremlin-1 Inhibition Restored Pulmonary Artery Diameter in Chronic Hypoxia

In Study 1, B-mode ultrasound imaging of the mouse heart in cross-section revealed that a 4 week exposure to hypoxia reduced PA CSA in saline-treated mice by ˜28% as compared to normoxic saline-treated mice (Table 3). Treatment with the isotype control antibody did not significantly affect PA CSA values from those observed in the hypoxic saline-treated mice. Treatment with the anti-Gremlin-1 antibody resulted in PA CSA sizes that were ˜46% larger than those measured for hypoxic isotype control antibody-treated mice, and this calculated PA CSA from the hypoxic anti-Gremlin-1-treated group was similar to that of the normoxic saline-treated mice group. Thus, the anti-Gremlin-1 antibody was able to restore pulmonary artery diameter in hypoxia.

In Study 2, B-mode ultrasound imaging of the mouse heart in cross-section revealed that a 6 week exposure to hypoxia reduced PA CSA by ˜32% in saline-treated mice relative to normoxic saline-treated mice (Table 3). The PA CSA values for isotype control antibody-treated animals were similar to saline-treated in hypoxia. Treatment with the anti-Gremlin-1 antibody at a lower concentration of 10 mg/kg resulted in a calculated PA CSA value that was 21% larger (significant) than the values calculated for the isotype control antibody treatment. The higher concentrations, 25 and 40 mg/kg, of anti-Gremlin-1 resulted in PA CSA values that were similar to those measured in the normoxic saline-treated mice and were significantly greater than the isotype control antibody treatment. These results demonstrate a dose dependent effect of anti-Gremlin-1 antibody on resolving the pulmonary artery diameter change induced by chronic hypoxia.

### Gremlin-1 Inhibition Restored Right Ventricular Cardiac Function

In Study 1, ultrasound M-mode imaging of the pulsed wave VTI showed non-significant differences (up to 5% increase) in the velocity of blood flow through the pulmonary artery in animals exposed to chronic hypoxia (data not shown). As shown in Table 3, calculated right ventricular stroke volumes (product of VTI and PA CSA) for both saline- and isotype control-treated mice were significantly reduced by 23-30% with exposure to hypoxia. Treatment with the anti-Gremlin-1 antibody resulted in a reversal of the reduction of right ventricular stroke volumes to values similar to that of normoxic saline-treated mice. This would imply that Gremlin-1 inhibition can restore stroke volume in hypoxia. Heart rate, which was measured and found not to be significantly different among groups, was used to determine right ventricular cardiac output. Right ventricular cardiac output was found to be significantly lower in animals exposed to chronic hypoxia by 21% relative to normoxic saline-treated mice. In comparison to hypoxic isotype control antibody treatment, measured cardiac output from hypoxic anti-Gremlin-1-treated mice was ˜48% greater; this value was 7% higher than the measured value in the normoxic saline-treated group, indicating that Gremlin-1 inhibition restored cardiac output in hypoxia. Collectively, these ultrasound results demonstrate that anti-Gremlin-1 antibody treatment in chronic hypoxia improves cardiac stroke volume and output with minimal changes to heart rate.

In Study 2, ultrasound M-mode imaging of the pulsed wave VTI revealed no significant differences (up to an 11% increase) in blood flow velocity through the pulmonary artery for animals treated with saline under normoxic versus hypoxic conditions. As shown in Table 3, hypoxia reduced the stroke volume by 32% in saline-treated animals when compared to normoxic mice. Compared to the hypoxic saline-treated group, the stroke volume calculated for the isotype control-treated group was 16% larger (non-significant) and because of this, comparisons to values from animals treated with either 10 or 40 mg/kg of anti-Gremlin-1 antibody were not statistically significant despite values that were 26-41% greater than values for the hypoxic saline-treated group and were comparable to values in the normoxic saline-treated group. Treatment with anti-Gremlin-1 antibody at 25 mg/kg resulted in average stroke volumes similar to values calculated for normoxic saline-treated mice, and these values were significantly greater than values calculated for isotype control antibody treatment by 38% (Table 3). Heart rate was measured and found to be comparable among different conditions. Six weeks of chronic hypoxia depressed right ventricular cardiac output by 35% in the saline-treated group, and use of the isotype control antibody had no effect on restoring cardiac output (27% reduction compared to normoxic saline). Use of 10 mg/kg of anti-Gremlin-1 antibody increased cardiac output (15% beyond the value measured for isotype control antibody treatment) in hypoxia but 16% below the values found in normoxic saline-treated mice. Use of anti-Gremlin-1 antibody at 25 or 40 mg/kg showed a benefit by increasing cardiac output by 30-35% more than treatment with isotype control antibody and was comparable to values found in normoxic saline-treated mice. Collectively, these data demonstrate that use of anti-Gremlin-1 antibody at high doses (25 or 40 mg/kg) in chronic hypoxia improves cardiac function.

TABLE 3
Average pulmonary artery cross-sectional area (PA CSA), stroke volume, heart
rate and right ventricular cardiac output measured at end of each study

| | | | | | | |
|---|---|---|---|---|---|---|
| | | | | | | Right Ventricular |
| | | | | Stroke Volume | Heart rate | cardiac output |
| | | | PA CSA (mm²) | (uL) | (beats/min) | (mL/min) |
| Group | Condition | Treatment | (Mean ± SEM) | (Mean ± SEM) | (Mean ± SEM) | (Mean ± SEM) |

Study 1

| | | | | | | |
|---|---|---|---|---|---|---|
| 1 | Normobaric | Saline | 1.817 ± 0.085 | 40.64 ± 1.69 | 464.2 ± 9.5 | 18.84 ± 0.83 |
| | normoxia | | | | | |
| 2 | Normobaric | Saline | 1.315 ± 0.052**** | 31.26 ± 1.09* | 475.1 ± 16.7 | 14.89 ± 0.82* |
| | hypoxia | | | | | |
| 3 | Normobaric | Isotype | 1.213 ± 0.039 | 28.55 ± 2.14 | 481.7 ± 20.5 | 13.53 ± 0.84 |
| | hypoxia | control | | | | |
| | | antibody | | | | |
| 4 | Normobaric | Anti-Gremlin- | 1.770 ± 0.058^{####} | 40.24 ± 3.51^{##} | 500.6 ± 12.7 | 20.09 ± 1.68^{###} |
| | hypoxia | 1 antibody | | | | |

Study 2

| | | | | | | |
|---|---|---|---|---|---|---|
| 1 | Normobaric | Saline | 1.711 ± 0.0392 | 34.86 ± 2.66 | 632.9 ± 7.3 | 22.06 ± 1.68 |
| | normoxia | | | | | |
| 2 | Normobaric | Saline | 1.169 ± 0.0269**** | 23.80 ± 1.89** | 612.9 ± 27.1 | 14.25 ± 0.86*** |
| | hypoxia | | | | | |
| 3 | Normobaric | Isotype | 1.205 ± 0.0281 | 27.51 ± 1.93 | 588.4 ± 18.7 | 16.07 ± 1.06 |
| | hypoxia | control | | | | |
| | | antibody | | | | |
| 4 | Normobaric | Anti-Gremlin- | 1.459 ± 0.0536^{##} | 30.57 ± 1.63 | 610.3 ± 23.8 | 18.59 ± 1.18 |
| | hypoxia | 1 antibody | | | | |
| | | (10 mg/kg) | | | | |
| 5 | Normobaric | Anti-Gremlin- | 1.718 ± 0.0863^{#####} | 38.09 ± 1.89^{##} | 572.7 ± 24.9 | 21.68 ± 1.21^{#} |
| | hypoxia | 1 antibody | | | | |
| | | (25 mg/kg) | | | | |
| 6 | Normobaric | Anti-Gremlin- | 1.727 ± 0.0640^{####} | 33.61 ± 2.76 | 631.4 ± 14.4 | 21.01 ± 1.51^{#} |
| | hypoxia | 1 antibody | | | | |
| | | (40 mg/kg) | | | | |

One-way ANOVA with Sidak's multiple comparison test: *, **, ***, **** for P < 0.05, 0.01, 0.001, 0.0001 vs. normobaric normoxia saline-treated; ^{#}, ^{##}, ^{###}, ^{####} for P < 0.01, 0.001, 0.0001 vs. normobaric hypoxia isotype control antibody-treated.

### Example 3. Anti-Gremlin-1 Antibody Treatment Restores Pulmonary Artery Diameter in a Sugen 5416/Chronic Hypoxia Mouse Model of Pulmonary Hypertension

To further evaluate the efficacy of the anti-GREM1 antibody, H4H6245P2, in treating pulmonary arterial hypertension, a vascular endothelial growth factor receptor antagonist, Sugen 5416/chronic hypoxia mouse model was used.

The following materials and methods were used for this study.

### Materials and Methods

Mice

Eleven to thirteen week old Taconic C57BL/6 mice were used. Mice were separated into treatment groups by weight such that starting body weights were similar among different groups. Cages were selected to either remain at about 21% O₂ (normobaric normoxia) or placed into 10% O₂ (normobaric hypoxia) chamber (a modified 3′ Semi-Rigid Isolator unit, Charles River) that maintained low O₂ levels with adjustment of N₂ flow to a steady intake of room air. Mice were administered Sugen5416 (Sigma, Cat#58442; VEGFR inhibitor subcutaneously at 20 mg/kg weekly for 6 weeks) and drugs or saline starting on day 21. A group of mice (n=10) housed in normobaric normoxia cages were subcutaneously administered saline at 5 mL/kg twice per week for three weeks, while mice housed in normobaric hypoxia cages were separated into 5 treatment groups including a group of mice (n=10) subcutaneously treated with saline at 5 mL/kg twice per week for three weeks, a group of mice (n=9) orally administered Bosentan (Sequoia Research Products Cat SRP02325b) at 300 mg/kg every day for three weeks, a group of mice (n=10) subcutaneously administered an isotype control antibody at 25 mg/kg twice per week for three weeks, a group of mice (n=10) subcutaneously treated with the anti-Gremlin-1 antibody at 25 mg/kg twice a week for three weeks, a group of mice (n=9) subcutaneously treated with an anti-Gremlin-1 antibody at 25 mg/kg twice a week for three weeks and orally administered Bosentan at 300 mg/kg every day for three weeks. Experimental dosing and treatment protocol for groups of mice are shown in Table 4.

TABLE 4
Therapeutic dosing and treatment protocol for each group in Sugen5416/chronic hypoxia mouse model study
Study 3: 6 weeks of Sugen5416/hypoxia with drug dosing beginning after 21 days in hypoxia.

| Group | Condition | Treatment | Dosage | Frequency | Route | “n” size |
|---|---|---|---|---|---|---|
| 1 | Normobaric normoxia + | Saline | 5 mL/kg | 2x/wk | SC | 10 |
| | Sugen5416 (20 mg/kg SC, | | | | | |
| | weekly) | | | | | |
| 2 | Normobaric hypoxia + | Saline | 5 mL/kg | 2x/wk | SC | 10 |
| | Sugen5416 (20 mg/kg SC, | | | | | |
| | weekly) | | | | | |
| 3 | Normobaric hypoxia + | Bosentan | 300 mg/kg | Daily | PO | 9 |
| | Sugen5416 (20 mg/kg SC, | | | | | |
| | weekly) | | | | | |
| 4 | Normobaric hypoxia + | Isotype control | 25 mg/kg | 2x/wk | SC | 10 |
| | Sugen5416 (20 mg/kg SC, | antibody | | | | |
| | weekly) | | | | | |
| 5 | Sugen5416 20 mg/kg SC, | Anti-Gremlin-1 | 25 mg/kg | 2x/wk | SC | 10 |
| | weekly Normobaric hypoxia | antibody | | | | |
| 6 | Normobaric hypoxia + | Anti-Gremlin-1 | Ab: | Ab: | Ab: SC | 9 |
| | Sugen5416 (20 mg/kg SC, | antibody + | 25 mg/kg | 2x/wk | Bosentan: | |
| | weekly) | Bosentan | Bosentan: | Bosentan: | PO | |
| | | | 300 mg/kg | Daily | | |

SC = subcutaneous
PO = per os

Ultrasound Assessment and Analysis

On the last day of the study, pulmonary artery size and right ventricular function and dimensions were assessed in each mouse using a high frequency ultrasound system (Vevo 2100, VisualSonics). For the assessment, mice were anesthetized (with 1.5% isoflurane at a rate of 1.0 cc/mL of medical grade air) and their temperature was monitored with a rectal temperature probe and held at approximately 37° C. with a heated platform (MouseMonitorS, Indus Instruments) and a warming lamp. Both brightness-mode (B-mode) and motion-mode (M-mode) imaging were used. B-mode imaging of the mouse heart in cross-section was used to determine pulmonary artery cross-sectional area (PA CSA) at the level of the pulmonary valve. M-mode imaging was used to determine the pulsed wave velocity time integral (VTI), which is derived from the area under the curve of representative Doppler tracings of blood flow through the pulmonary artery. Right ventricular stroke volume (RV SV) was calculated from the product of PA CSA and VTI. Right ventricular cardiac output (RV CO) was calculated from the product of SV and heart rate (HR). M-mode imaging was used to determine right ventricular free wall (RVFW) thickness during diastole and systole. Animals were returned to their home cages before right ventricular pressure assessment.

Right Ventricular Pressure Assessment

Right ventricular pressure was subsequently assessed for all treatment groups. Mice were anesthetized with isoflurane and were kept at approximately 37° C. using a heated platform (Heated Hard Pad 1, Braintree Scientific) and circulating heated water pump (T/Pump Classic, Gaymar Industries). The neck area for each mouse was prepared for surgery by depilating over the Right Common Carotid Artery and right Jugular Vein. An incision was made and the right Jugular Vein was isolated with care as to not damage the Carotid Artery and/or the Vagus nerve. A piece of 5-0 silk suture was placed under the isolated Jugular Vein to allow for retraction of the vessel cranially, then a 30-gauge needle was used to introduce a hole into the Jugular Vein. A pressure catheter (Micro-tip catheter transducer SPR-1000, Millar Instruments, Inc.) was inserted into the opening of the Jugular Vein and advanced past the right atrium into the right ventricle. The catheter was connected to pressure/volume instrument (MPVS-300, Millar Instruments, Inc.) that measured heart rate as well as both diastolic and systolic right ventricular pressures. These parameters were digitally acquired using a data acquisition system (PowerLab 4/35, ADInstruments). LabChart Pro 7.0 software (ADInstruments) was used to analyze right ventricular pressures. Readings were quantified from a 60 second interval of the pressure tracing (following a 2 minute period of recording to allow for pressure stabilization). The parameters analyzed were right ventricular systolic pressures (RVSP), heart rate (HR) and rate of right ventricular pressure rise (dP/dt max).

Serum/Tissue Collection and Assessment of Right Ventricular Hypertrophy

Following completion of right ventricular pressure measurement, the catheter was removed and each animal was sacrificed. The abdomen was opened and blood was drawn from the vena cava for hematocrit assessment and serum collection. The thoracic cavity was then opened and the middle lobe of the right lung was ligated with 5-0 silk suture, excised, placed in RNA later (Sigma-Aldrich, cat #R0901) and frozen 24 hours later at −80° C. The heart was excised from each animal, and the right ventricle (RV) was carefully cut away from the left ventricle and septum (LV+S). Both pieces of heart tissue were separately weighed on a microbalance (AJ000, Mettler) to calculate the index of RV hypertrophy [RV/(LV+S); Fulton Index].

Half of the animals from each treatment group had the lungs perfused at 20-25 mmHg with phosphate buffered solution (PBS, pH 7.4), then fixed with 10% neutral-buffered formalin (NBF). Lungs remained in 10% NBF for 24 hours before being placed into 70% ethanol for at least 48 hours, before tissue processing and paraffin embedding. For animals that did not undergo perfusion-fixation of the lung, the right inferior lobe was ligated with 5-0 silk suture before being excised, weighed and frozen in liquid N₂.

### Results

### Gremlin-1 Inhibition Restored Pulmonary Artery Diameter in Sugen5416/Hypoxia.

As shown in Table 5, B-mode ultrasound imaging of the mouse heart in cross-section revealed that a 6-week exposure to Sugen5416/hypoxia reduced PA CSA by 29% in saline-treated mice (comparison to normoxic mice). In hypoxia, PA CSA values for isotype control antibody-treated animals were similar to saline-treated. Use of the endothelin receptor antagonist Bosentan resulted in PA CSA values that were ˜43% greater than the hypoxic saline-treated group (significant) yet similar to those measured in normoxia. Similarly, use of the anti-Gremlin-1 antibody resulted in PA CSA values that were significantly greater (by 28%) than values measured in the isotype control antibody treatment group yet similar to those observed in normoxic saline-treated mice. However, use of combination Bosentan and anti-Gremlin-1 antibody had little effect on PA CSA and was similar to values found for saline or isotype control antibody treatment in hypoxia.

TABLE 5
Average pulmonary artery cross-sectional area (PA CSA), stroke volume and
right ventricular cardiac output of treatment groups at end of study

| | | | | | | Right Ventricular |
|---|---|---|---|---|---|---|
| | | | | Stroke Volume | Heart rate | cardiac output |
| | | | PA CSA (mm²) | (ul) | (beats/min) | (ml/min) |
| Group | Condition | Treatment | (Ave ± SEM) | (Ave ± SEM) | (Ave ± SEM) | (Ave ± SEM) |
| 1 | Normobaric | Saline | 1.683 ± 0.063 | 32.31 ± 2.20 | 443.0 ± 11.7 | 14.20 ± 0.93 |
| | normoxia + | | | | | |
| | Sugen5416 | | | | | |
| | (20 mg/kg | | | | | |
| | SC, weekly) | | | | | |
| 2 | Normobaric | Saline | 1.202 ± 0.062** | 23.09 ± 1.61 | 501.7 ± 22.4 | 11.43 ± 0.71 |
| | hypoxia + | | | | | |
| | Sugen5416 | | | | | |
| | (20 mg/kg | | | | | |
| | SC, weekly) | | | | | |
| 3 | Normobaric | Bosentan | 1.724 ± 0.074^{%%%} | 35.51 ± 3.22^{%%} | 509.7 ± 18.9 | 17.81 ± 1.46^{%%} |
| | hypoxia + | | | | | |
| | Sugen5416 | | | | | |
| | (20 mg/kg | | | | | |
| | SC, weekly) | | | | | |
| 4 | Normobaric | Isotype | 1.226 ± 0.051 | 21.71 ± 2.39 | 553.7 ± 19.6 | 11.81 ± 1.16 |
| | hypoxia + | control | | | | |
| | Sugen5416 | antibody | | | | |
| | (20 mg/kg | | | | | |
| | SC, weekly) | | | | | |
| 5 | Sugen5416 | Anti- | 1.565 ± 0.147^{#} | 30.23 ± 3.71 | 524.7 ± 18.3 | 16.19 ± 2.22 |
| | 20 mg/kg | Gremlin-1 | | | | |
| | SC, weekly | antibody | | | | |
| | Normobaric | | | | | |
| | hypoxia | | | | | |
| 6 | Normobaric | Anti- | 1.227 ± 0.075 | 23.14 ± 1.62 | 511.0 ± 14.8 | 11.92 ± 01.00 |
| | hypoxia + | Gremlin-1 | | | | |
| | Sugen5416 | antibody + | | | | |
| | (20 mg/kg | Bosentan | | | | |
| | SC, weekly) | | | | | |

One-way ANOVA with Sidak's multiple comparison test: ** for P < 0.01 vs. normobaric hypoxia saline-treated; ^{%%}, ^{%%%} for P < 0.01, 0.001 vs. normobaric saline-treated; ^{#} for P < 0.05 vs. normobaric hypoxia isotype control antibody-treated.

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

We claim:

1. A method of treating a subject having pulmonary arterial hypertension (PAH), comprising
administering to the subject a therapeutically effective amount of an anti-gremlin-1 (GREM1) antibody, or antigen-binding fragment thereof,
wherein the therapeutic effect of administration of the anti-GREM1 antibody, or antigen-binding fragment thereof, to the subject is selected from the group consisting of
inhibiting thickening of the pulmonary artery in the subject;
increasing stroke volume in the subject;
increasing right ventricle cardiac output in the subject; and
extending survival time of the subject, thereby treating the subject having PAH.

2. The method of claim 1, wherein the subject is human.

3. The method of claim 1, wherein the subject has Group I (WHO) PAH.

4. The method of claim 1, wherein the method further comprises administering to the subject at least one additional therapeutic agent.

5. The method of claim 4, wherein the therapeutic agent is selected from the group consisting of an anticoagulant, a diuretic, a cardiac glycoside, a calcium channel blocker, a vasodilator, a prostacyclin analogue, an endothelium antagonist, a phosphodiesterase inhibitor, an endopeptidase inhibitor, a lipid lowering agent, and a thromboxane inhibitor.

6. The method of claim 1, wherein the antibody, or antigen-binding fragment thereof, blocks GREM1 binding to one of bone morphogenetic protein-2 (BMP2), BMP4, BMP7 or heparin.

7. The method of claim 1, wherein the antibody, or antigen-binding fragment thereof, exhibits one or more properties selected from the group consisting of:
(a) binds GREM1 at 37° C. with a binding dissociation equilibrium constant (K_{D}) of less than about 275 nM as measured by surface plasmon resonance;
(b) binds to GREM1 at 37° C. with a dissociative half-life (t½) of greater than about 3 minutes as measured by surface plasmon resonance;
(c) binds GREM1 at 25° C. with a K_{D} of less than about 280 nM as measured by surface plasmon resonance;
(d) binds to GREM1 at 25° C. with a t1/2 of greater than about 2 minutes as measured by surface plasmon resonance;
(e) blocks GREM1 binding to BMP4 with an IC₅₀ of less than about 1.9 nM as measured in a competition ELISA assay at 25° C.;
(f) blocks GREM1-mediated inhibition of BMP signaling and promotes cell differentiation; and
(g) blocks GREM1 binding to heparin.

8. The method of claim 1, wherein the antibody, or antigen-binding fragment thereof, competes for specific binding to GREM1 with an antibody, or antigen-binding fragment thereof, comprising the complementarity determining regions (CDRs) of a heavy chain variable region (HCVR), wherein the HCVR has an amino acid sequence selected from the group consisting of SEQ ID NOs: 2, 18, 34, 50, 66, 82, 98, 1 14, 130, 146, 162, 178, 194, 210, 226, 242, 258, 274, 290, 306, 322, 338, 354, 370, 386, 402, 418, 434, 450, 466, 482, 498, 514, 530, 546, 562, and 578.

9. The method of claim 1, wherein the antibody, or antigen-binding fragment thereof, competes for specific binding to GREM1 with an antibody, or antigen-binding fragment thereof, comprising the CDRs of a light chain variable region (LCVR), wherein the LCVR has an amino acid sequence selected from the group consisting of SEQ ID NOs: 10, 26, 42, 58, 74, 90, 106, 122, 138, 154, 170, 186, 202, 218, 234, 250, 266, 282, 298, 314, 330, 346, 362, 378, 394, 410, 426, 442, 458, 474, 490, 506, 522, 538, 554, 570, and 586.

10. The method of claim 1, wherein the antibody, or antigen-binding fragment thereof, comprises three heavy chain complementarity determining regions (CDRs) (HCDR1, HCDR2 and HCDR3) contained within any one of the heavy chain variable region (HCVR) sequences selected from the group consisting of SEQ ID NOs: 2, 18, 34, 50, 66, 82, 98, 1 14, 130, 146, 162, 178, 194, 210, 226, 242, 258, 274, 290, 306, 322, 338, 354, 370, 386, 402, 418, 434, 450, 466, 482, 498, 514, 530, 546, 562, and 578; and three light chain CDRs (LCDR1, LCDR2 and LCDR3) contained within any one of the light chain variable region (LCVR) sequences selected from the group consisting of SEQ ID NOs: 10, 26, 42, 58, 74, 90, 106, 122, 138, 154, 170, 186, 202, 218, 234, 250, 266, 282, 298, 314, 330, 346, 362, 378, 394, 410, 426, 442, 458, 474, 490, 506, 522, 538, 554, 570, and 586.

11. The method of claim 10, wherein the antibody, or antigen-binding fragment thereof, comprises a HCVR having an amino acid sequence selected from the group consisting of SEQ ID NOs: 2, 18, 34, 50, 66, 82, 98, 1 14, 130, 146, 162, 178, 194, 210, 226, 242, 258, 274, 290, 306, 322, 338, 354, 370, 386, 402, 418, 434, 450, 466, 482, 498, 514, 530, 546, 562, and 578.

12. The method of claim 10, wherein the antibody, or antigen-binding fragment thereof, comprises a LCVR having an amino acid sequence selected from the group consisting of SEQ ID NOs: 10, 26, 42, 58, 74, 90, 106, 122, 138, 154, 170, 186, 202, 218, 234, 250, 266, 282, 298, 314, 330, 346, 362, 378, 394, 410, 426, 442, 458, 474, 490, 506, 522, 538, 554, 570, and 586.

13. The method of claim 10, wherein the antibody, or antigen-binding fragment thereof, comprises: (a) a HCVR having an amino acid sequence selected from the group consisting of SEQ ID NOs: 2, 18, 34, 50, 66, 82, 98, 1 14, 130, 146, 162, 178, 194, 210, 226, 242, 258, 274, 290, 306, 322, 338, 354, 370, 386, 402, 418, 434, 450, 466, 482, 498, 514, 530, 546, 562, and 578; and (b) a LCVR having an amino acid sequence selected from the group consisting of SEQ ID NO: 10, 26, 42, 58, 74, 90, 106, 122, 138, 154, 170, 186, 202, 218, 234, 250, 266, 282, 298, 314, 330, 346, 362, 378, 394, 410, 426, 442, 458, 474, 490, 506, 522, 538, 554, 570, and 586.

14. The method of claim 10, wherein the antibody, or antigen-binding fragment thereof, comprises
(a) a HCDR1 domain having an amino acid sequence selected from the group consisting of SEQ ID NOs: 4, 20, 36, 52, 68, 84, 100, 1 16, 132, 148, 164, 180, 196, 212, 228, 244, 260, 276, 292, 308, 324, 340, 356, 372, 388, 404, 420, 436, 452, 468, 484, 500, 516, 532, 548, 564, and 580;
(b) a HCDR2 domain having an amino acid sequence selected from the group consisting of SEQ ID NOs: 6, 22, 38, 54, 70, 86, 102, 1 18, 134, 150, 166, 182, 198, 214, 230, 246, 262, 278, 294, 310, 326, 342, 358, 374, 390, 406, 422, 438, 454, 470, 486, 502, 518, 534, 550, 566, and 582;
(c) a HCDR3 domain having an amino acid sequence selected from the group consisting of SEQ ID NOs: 8, 24, 40, 56, 72, 88, 104, 120, 136, 152, 168, 184, 200, 216, 232, 248, 264, 280, 296, 312, 328, 344, 360, 376, 392, 408, 424, 440, 456, 472, 488, 504, 520, 536, 552, 568, and 584;
(d) a LCDR1 domain having an amino acid sequence selected from the group consisting of SEQ ID NOs: 12, 28, 44, 60, 76, 92, 108, 124, 140, 156, 172, 188, 204, 220, 236, 252, 268, 284, 300, 316, 332, 348, 364, 380, 396, 412, 428, 444, 460, 476, 492, 508, 524, 540, 556, 572, and 588;
(e) a LCDR2 domain having an amino acid sequence selected from the group consisting of SEQ ID NOs: 14, 30, 46, 62, 78, 94, 1 10, 126, 142, 158, 174, 190, 206, 222, 238, 254, 270, 286, 302, 318, 334, 350, 366, 382, 398, 414, 430, 446, 462, 478, 494, 510, 526, 542, 558, 574, and 590; and/or
(f) a LCDR3 domain having an amino acid sequence selected from the group consisting of SEQ ID NOs: 16, 32, 48, 64, 80, 96, 1 12, 128, 144, 160, 176, 192, 208, 224, 240, 256, 272, 288, 304, 320, 336, 352, 368, 384, 400, 416, 432, 448, 464, 480, 496, 512, 528, 544, 560, 576, and 592.

15. The method of claim 10, wherein the antibody, or antigen-binding fragment thereof, comprises a HCVR/LCVR amino acid sequence pair selected from the group consisting of SEQ ID NOs: 2/10, 18/26, 34/42, 50/58, 66/74, 82/90, 98/106, 1 14/122, 130/138, 146/154, 162/170, 178/186, 194/202, 210/218, 226/234, 242/250, 258/266, 274/282, 290/298, 306/314, 322/330, 338/346, 354/362, 370/378, 386/394, 402/410, 418/426, 434/442, 450/458, 466/474, 482/490, 498/506, 514/522, 530/538, 546/554, 562/570, and 578/586.

16. The method of claim 1, wherein the antibody, or antigen-binding fragment thereof, binds the same epitope on GREM1 as an antibody or antigen-binding fragment comprising the complementarity determining regions (CDRs) of a heavy chain variable region (HCVR), wherein the HCVR has an amino acid sequence selected from the group consisting of SEQ ID NOs: 2, 18, 34, 50, 66, 82, 98, 1 14, 130, 146, 162, 178, 194, 210, 226, 242, 258, 274, 290, 306, 322, 338, 354, 370, 386, 402, 418, 434, 450, 466, 482, 498, 514, 530, 546, 562, and 578; and the CDRs of a light chain variable region (LCVR), wherein the LCVR has an amino acid sequence selected from the group consisting of SEQ ID NOs: 10, 26, 42, 58, 74, 90, 106, 122, 138, 154, 170, 186, 202, 218, 234, 250, 266, 282, 298, 314, 330, 346, 362, 378, 394, 410, 426, 442, 458, 474, 490, 506, 522, 538, 554, 570, and 586.


SEQUENCE LISTING (595 sequences)

SEQ ID NO: 1 - DNA, 357 bp, Artificial Sequence
  Synthetic
caggtgcagctggtggagtctgggggaggcgtggtccagcctgggaggtccctgagactctcctgtgcagcgtctggattcaccttcagcagctatggcatgcactgggtccgccaggctccaggcaaggggctggagtgggtggcaattatatggaatgatggaagtaataaatactatgtagactccgtgaagggccgattcaccatctccagagccaattccaagaacacgctgtatctgcaaatgaacagcctgagagccgaagacacggctgtgtattactgtgcgagagacggactggaacctgatgcttttgatatctggggccaagggacaatggtcaccgtttcttca

SEQ ID NO: 2 - protein, 119 aa, Artificial Sequence
  Synthetic
QVQLVESGGGVVQPGRSLRLSCAASGFTFSSYGMHWVRQAPGKGLEWVAIIWNDGSNKYYVDSVKGRFTISRANSKNTLYLQMNSLRAEDTAVYYCARDGLEPDAFDIWGQGTMVTVSS

SEQ ID NO: 3 - DNA, 24 bp, Artificial Sequence
  Synthetic
ggattcaccttcagcagctatggc

SEQ ID NO: 4 - protein, 8 aa, Artificial Sequence
  Synthetic
GFTFSSYG

SEQ ID NO: 5 - DNA, 24 bp, Artificial Sequence
  Synthetic
atatggaatgatggaagtaataaa

SEQ ID NO: 6 - protein, 8 aa, Artificial Sequence
  Synthetic
IWNDGSNK

SEQ ID NO: 7 - DNA, 36 bp, Artificial Sequence
  Synthetic
gcgagagacggactggaacctgatgcttttgatatc

SEQ ID NO: 8 - protein, 12 aa, Artificial Sequence
  Synthetic
ARDGLEPDAFDI

SEQ ID NO: 9 - DNA, 324 bp, Artificial Sequence
  Synthetic
gaaattgtgttgacacagtctccagccaccctgtctttgtctccaggggaaagagccaccctctcctgcagggccagtcagagtgttagcagcttcttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccgtacacttttggccaggggaccaagctggagatcaaa

SEQ ID NO: 10 - protein, 108 aa, Artificial Sequence
  Synthetic
EIVLTQSPATLSLSPGERATLSCRASQSVSSFLAWYQQKPGQAPRLLIYDASNRATGIPARFSGSGSGTDFTLTISSLEPEDFAVYYCQQRSNWPPYTFGQGTKLEIK

SEQ ID NO: 11 - DNA, 18 bp, Artificial Sequence
  Synthetic
cagagtgttagcagcttc

SEQ ID NO: 12 - protein, 6 aa, Artificial Sequence
  Synthetic
QSVSSF

SEQ ID NO: 13 - DNA, 9 bp, Artificial Sequence
  Synthetic
gatgcatcc

SEQ ID NO: 14 - protein, 3 aa, Artificial Sequence
  Synthetic
DAS

SEQ ID NO: 15 - DNA, 30 bp, Artificial Sequence
  Synthetic
cagcagcgtagcaactggcctccgtacact

SEQ ID NO: 16 - protein, 10 aa, Artificial Sequence
  Synthetic
QQRSNWPPYT

SEQ ID NO: 17 - DNA, 360 bp, Artificial Sequence
  Synthetic
gaggtgcagctggtggagtctgggggcggcctggtcaagcctggggggtccctgagactctcctgtgcagcctctggattcaccttcagtacctacagcatgaactgggtccgccaggctccagggaaggggctggagtgggtctcatccattagtagtggtagtagttacatatactacacagactcagtgaagggccgattcaccatctccagagacaacgccaagaactcactgtatctgcaaatgaacagcctgagagccgaggacacggctgtgtattactgtgcgagattcgggagctactactacttcggtttcgacgtctggggccaagggaccacggtcaccgtctcctca

SEQ ID NO: 18 - protein, 120 aa, Artificial Sequence
  Synthetic
EVQLVESGGGLVKPGGSLRLSCAASGFTFSTYSMNWVRQAPGKGLEWVSSISSGSSYIYYTDSVKGRFTISRDNAKNSLYLQMNSLRAEDTAVYYCARFGSYYYFGFDVWGQGTTVTVSS

SEQ ID NO: 19 - DNA, 24 bp, Artificial Sequence
  Synthetic
ggattcaccttcagtacctacagc

SEQ ID NO: 20 - protein, 8 aa, Artificial Sequence
  Synthetic
GFTFSTYS

SEQ ID NO: 21 - DNA, 24 bp, Artificial Sequence
  Synthetic
attagtagtggtagtagttacata

SEQ ID NO: 22 - protein, 8 aa, Artificial Sequence
  Synthetic
ISSGSSYI

SEQ ID NO: 23 - DNA, 39 bp, Artificial Sequence
  Synthetic
gcgagattcgggagctactactacttcggtttcgacgtc

SEQ ID NO: 24 - protein, 13 aa, Artificial Sequence
  Synthetic
ARFGSYYYFGFDV

SEQ ID NO: 25 - DNA, 321 bp, Artificial Sequence
  Synthetic
gacatccagatgacccagtctccatcctccctgtctgcatctgtaggagacagagtcaccatcacttgccgggcgagtcagggcattagcaattatttagcctggtatcagcagaaaccagggaaagttcctaaactcctgatcttttctgcatccactttgcaatcaggggtcccatctcggttcagtggcagtggatctgggccagatttcactctcaccgtcagcagcctgcagcctgaagatgttgcaacttattactgtcaaaagtataacagtgccccattcgctttcggccctgggaccaaagtggatatcaaa

SEQ ID NO: 26 - protein, 107 aa, Artificial Sequence
  Synthetic
DIQMTQSPSSLSASVGDRVTITCRASQGISNYLAWYQQKPGKVPKLLIFSASTLQSGVPSRFSGSGSGPDFTLTVSSLQPEDVATYYCQKYNSAPFAFGPGTKVDIK

SEQ ID NO: 27 - DNA, 18 bp, Artificial Sequence
  Synthetic
cagggcattagcaattat

SEQ ID NO: 28 - protein, 6 aa, Artificial Sequence
  Synthetic
QGISNY

SEQ ID NO: 29 - DNA, 9 bp, Artificial Sequence
  Synthetic
tctgcatcc

SEQ ID NO: 30 - protein, 3 aa, Artificial Sequence
  Synthetic
SAS

SEQ ID NO: 31 - DNA, 27 bp, Artificial Sequence
  Synthetic
caaaagtataacagtgccccattcgct

SEQ ID NO: 32 - protein, 9 aa, Artificial Sequence
  Synthetic
QKYNSAPFA

SEQ ID NO: 33 - DNA, 354 bp, Artificial Sequence
  Synthetic
gaggtgcagctggtggagtctgggggaggcctggtccagcctggggggtccctgagactctcctgtgcagcctctggattcaccttcagtagttatagcatgaactgggtccgccaggctccagggaaggggctggagtgggtctcatccataagtagtagtagtaattacataaactacgcagactctattaagggccgattcaccatctccagagacaacgccaagaactcactatatctacaaatgaacagcctgagagccgaggatacggctgtgtattactgtgcgagagttaattgggactacccctttgactgctggggccggggaaccctggtcaccgtctcctca

SEQ ID NO: 34 - protein, 118 aa, Artificial Sequence
  Synthetic
EVQLVESGGGLVQPGGSLRLSCAASGFTFSSYSMNWVRQAPGKGLEWVSSISSSSNYINYADSIKGRFTISRDNAKNSLYLQMNSLRAEDTAVYYCARVNWDYPFDCWGRGTLVTVSS

SEQ ID NO: 35 - DNA, 24 bp, Artificial Sequence
  Synthetic
ggattcaccttcagtagttatagc

SEQ ID NO: 36 - protein, 8 aa, Artificial Sequence
  Synthetic
GFTFSSYS

SEQ ID NO: 37 - DNA, 24 bp, Artificial Sequence
  Synthetic
ataagtagtagtagtaattacata

SEQ ID NO: 38 - protein, 8 aa, Artificial Sequence
  Synthetic
ISSSSNYI

SEQ ID NO: 39 - DNA, 33 bp, Artificial Sequence
  Synthetic
gcgagagttaattgggactacccctttgactgc

SEQ ID NO: 40 - protein, 11 aa, Artificial Sequence
  Synthetic
ARVNWDYPFDC

SEQ ID NO: 41 - DNA, 321 bp, Artificial Sequence
  Synthetic
gacatccagatgacccagtctccatcctccctgtctgcatctgtaggagacagagtcaccatcacttgccgggcgagtcaggacattagacattatttagtctggtatcagcagaaaccagggaaagttcctaagctcctgatctatgctgcatccactttgcaatcaggggtcccatctcggttcagtggcagtggatctgggacagatttcattctcaccatcagcagcctgcagcctgaagatgttgcaacttattactgtcaaaagtataacagtgccccattcactttcggccctgggaccaaagtggatatcaaa

SEQ ID NO: 42 - protein, 107 aa, Artificial Sequence
  Synthetic
DIQMTQSPSSLSASVGDRVTITCRASQDIRHYLVWYQQKPGKVPKLLIYAASTLQSGVPSRFSGSGSGTDFILTISSLQPEDVATYYCQKYNSAPFTFGPGTKVDIK

SEQ ID NO: 43 - DNA, 18 bp, Artificial Sequence
  Synthetic
caggacattagacattat

SEQ ID NO: 44 - protein, 6 aa, Artificial Sequence
  Synthetic
QDIRHY

SEQ ID NO: 45 - DNA, 9 bp, Artificial Sequence
  Synthetic
gctgcatcc

SEQ ID NO: 46 - protein, 3 aa, Artificial Sequence
  Synthetic
AAS

SEQ ID NO: 47 - DNA, 27 bp, Artificial Sequence
  Synthetic
caaaagtataacagtgccccattcact

SEQ ID NO: 48 - protein, 9 aa, Artificial Sequence
  Synthetic
QKYNSAPFT

SEQ ID NO: 49 - DNA, 366 bp, Artificial Sequence
  Synthetic
gaggtgcagctgttggagtctgggggaggcttggtacagccgggggggtccctgagactctcctgtgcagcctctggattcacctttagcagctatgtcatgaactgggtccgccaggctccagggaaggggctggagtgggtctcagctattagcggaagtggtggtagcacatactacgcagactccgtgaagggccggtccaccatctccagagacaattccaagaacacactgtatctgcaaatgaatagcctgagagccgaggacacggccatatattattgtgcgaaaggggatatagcagcaattgtctttgatgcttttgatatctggggccaagggacagtggtcaccgtctcttca

SEQ ID NO: 50 - protein, 122 aa, Artificial Sequence
  Synthetic
EVQLLESGGGLVQPGGSLRLSCAASGFTFSSYVMNWVRQAPGKGLEWVSAISGSGGSTYYADSVKGRSTISRDNSKNTLYLQMNSLRAEDTAIYYCAKGDIAAIVFDAFDIWGQGTVVTVSS

SEQ ID NO: 51 - DNA, 24 bp, Artificial Sequence
  Synthetic
ggattcacctttagcagctatgtc

SEQ ID NO: 52 - protein, 8 aa, Artificial Sequence
  Synthetic
GFTFSSYV

SEQ ID NO: 53 - DNA, 24 bp, Artificial Sequence
  Synthetic
attagcggaagtggtggtagcaca

SEQ ID NO: 54 - protein, 8 aa, Artificial Sequence
  Synthetic
ISGSGGST

SEQ ID NO: 55 - DNA, 45 bp, Artificial Sequence
  Synthetic
gcgaaaggggatatagcagcaattgtctttgatgcttttgatatc

SEQ ID NO: 56 - protein, 15 aa, Artificial Sequence
  Synthetic
AKGDIAAIVFDAFDI

SEQ ID NO: 57 - DNA, 321 bp, Artificial Sequence
  Synthetic
gacatccagatgacccagtctccatcctccctgtctgcatctgtaggagacagagtcaccatcacttgccaggcgagtcaggacattagcagctgtttaaattggtatcaacacaaaccagggaaagcccctaagctcctgatctacgatgcatcctatttggaaacaggggtcccatcaaggttcagtggaagtggatctgggacagattttactttcaccatcagcagcctgcagcctgaagatattgcaacatattactgtcaacagtatgataatctcccgtacacttttggccaggggaccaagctggagatcaaa

SEQ ID NO: 58 - protein, 107 aa, Artificial Sequence
  Synthetic
DIQMTQSPSSLSASVGDRVTITCQASQDISSCLNWYQHKPGKAPKLLIYDASYLETGVPSRFSGSGSGTDFTFTISSLQPEDIATYYCQQYDNLPYTFGQGTKLEIK

SEQ ID NO: 59 - DNA, 18 bp, Artificial Sequence
  Synthetic
caggacattagcagctgt

SEQ ID NO: 60 - protein, 6 aa, Artificial Sequence
  Synthetic
QDISSC

SEQ ID NO: 61 - DNA, 9 bp, Artificial Sequence
  Synthetic
gatgcatcc

SEQ ID NO: 62 - protein, 3 aa, Artificial Sequence
  Synthetic
DAS

SEQ ID NO: 63 - DNA, 27 bp, Artificial Sequence
  Synthetic
caacagtatgataatctcccgtacact

SEQ ID NO: 64 - protein, 9 aa, Artificial Sequence
  Synthetic
QQYDNLPYT

SEQ ID NO: 65 - DNA, 366 bp, Artificial Sequence; identical to SEQ ID NO: 49
  Synthetic

SEQ ID NO: 66 - protein, 122 aa, Artificial Sequence; identical to SEQ ID NO: 50
  Synthetic

SEQ ID NO: 67 - DNA, 24 bp, Artificial Sequence
  Synthetic
ggattcacctttagcagctatgtc

SEQ ID NO: 68 - protein, 8 aa, Artificial Sequence
  Synthetic
GFTFSSYV

SEQ ID NO: 69 - DNA, 24 bp, Artificial Sequence
  Synthetic
attagcggaagtggtggtagcaca

SEQ ID NO: 70 - protein, 8 aa, Artificial Sequence
  Synthetic
ISGSGGST

SEQ ID NO: 71 - DNA, 45 bp, Artificial Sequence; identical to SEQ ID NO: 55
  Synthetic

SEQ ID NO: 72 - protein, 15 aa, Artificial Sequence
  Synthetic
AKGDIAAIVFDAFDI

SEQ ID NO: 73 - DNA, 321 bp, Artificial Sequence
  Synthetic
gacatccagatgacccagtctccatcctccctgtctgcatctgtaggagacagagtcaccatcacttgccaggcgagtcaggacattagcagcgctttaaattggtatcaacacaaaccagggaaagcccctaagctcctgatctacgatgcatcctatttggaaacaggggtcccatcaaggttcagtggaagtggatctgggacagattttactttcaccatcagcagcctgcagcctgaagatattgcaacatattactgtcaacagtatgataatctcccgtacacttttggccaggggaccaagctggagatcaaa

SEQ ID NO: 74 - protein, 107 aa, Artificial Sequence
  Synthetic
DIQMTQSPSSLSASVGDRVTITCQASQDISSALNWYQHKPGKAPKLLIYDASYLETGVPSRFSGSGSGTDFTFTISSLQPEDIATYYCQQYDNLPYTFGQGTKLEIK

SEQ ID NO: 75 - DNA, 18 bp, Artificial Sequence
  Synthetic
caggacattagcagcgct

SEQ ID NO: 76 - protein, 6 aa, Artificial Sequence
  Synthetic
QDISSA

SEQ ID NO: 77 - DNA, 9 bp, Artificial Sequence
  Synthetic
gatgcatcc

SEQ ID NO: 78 - protein, 3 aa, Artificial Sequence
  Synthetic
DAS

SEQ ID NO: 79 - DNA, 27 bp, Artificial Sequence
  Synthetic
caacagtatgataatctcccgtacact

SEQ ID NO: 80 - protein, 9 aa, Artificial Sequence
  Synthetic
QQYDNLPYT

SEQ ID NO: 81 - DNA, 348 bp, Artificial Sequence
  Synthetic
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtgacaattatatggcatgatggaagtaataaatactatgcagactccgtgaagggccgattcaccatctccagagacaattccaagaacacgctgtatctgcaaatgaacagtgtgagagccgaggacacggctgtgtattactgtgcgagagacgaagatttttttgactactggggccagggaaccctggtcaccgtctcctca

SEQ ID NO: 82 - protein, 116 aa, Artificial Sequence
  Synthetic
QVQLVESGGGVVQPGRSLRLSCAASGFTFSSYGMHWVRQAPGKGLEWVTIIWHDGSNKYYADSVKGRFTISRDNSKNTLYLQMNSVRAEDTAVYYCARDEDFFDYWGQGTLVTVSS

SEQ ID NO: 83 - DNA, 24 bp, Artificial Sequence
  Synthetic
ggattcaccttcagtagctatggc

SEQ ID NO: 84 - protein, 8 aa, Artificial Sequence
  Synthetic
GFTFSSYG

SEQ ID NO: 85 - DNA, 24 bp, Artificial Sequence
  Synthetic
atatggcatgatggaagtaataaa

SEQ ID NO: 86 - protein, 8 aa, Artificial Sequence
  Synthetic
IWHDGSNK

SEQ ID NO: 87 - DNA, 27 bp, Artificial Sequence
  Synthetic
gcgagagacgaagatttttttgactac

SEQ ID NO: 88 - protein, 9 aa, Artificial Sequence
  Synthetic
ARDEDFFDY

SEQ ID NO: 89 - DNA, 324 bp, Artificial Sequence
  Synthetic
gaaattgtgatgacgcagtctccagccaccctgtctgtgtctccaggggaaaaagccaccctctcctgcagggccagtcagagtgttagtatcaacttagcctggtaccaacagaaacctggccaggctcccaggctcctcatctatgatgcatccaccagggccactggtatcccagccaggttcagtggcagtgggtctgggacagagttcactctcaccatcagcagcctgcagtctgaagattttgcagtttattactgtcagcagtataataactggcctccgtacacttttggccaggggactaagctggagatcaaa

SEQ ID NO: 90 - protein, 108 aa, Artificial Sequence
  Synthetic
EIVMTQSPATLSVSPGEKATLSCRASQSVSINLAWYQQKPGQAPRLLIYDASTRATGIPARFSGSGSGTEFTLTISSLQSEDFAVYYCQQYNNWPPYTFGQGTKLEIK

SEQ ID NO: 91 - DNA, 18 bp, Artificial Sequence
  Synthetic
cagagtgttagtatcaac

SEQ ID NO: 92 - protein, 6 aa, Artificial Sequence
  Synthetic
QSVSIN

SEQ ID NO: 93 - DNA, 9 bp, Artificial Sequence
  Synthetic
gatgcatcc

SEQ ID NO: 94 - protein, 3 aa, Artificial Sequence
  Synthetic
DAS

SEQ ID NO: 95 - DNA, 30 bp, Artificial Sequence
  Synthetic
cagcagtataataactggcctccgtacact

SEQ ID NO: 96 - protein, 10 aa, Artificial Sequence
  Synthetic
QQYNNWPPYT

SEQ ID NO: 97 - DNA, 366 bp, Artificial Sequence
  Synthetic
gaggtgcagctgttggagtctgggggaggcttggtacagccgggggggtccctgagactctcctgtgcagcctctggattcacctttagcagctatgtcatgaactgggtccgccaggctccagggaaggggctggagtgggtctcagctattagcggaagtggtggtagcacatcctacgcagactccgtgaagggccggtccaccatctccagagacaattccaagaacacactgtatctgcaaatgaatagcctgagagccgaggacacggccgtatattattgtgcgaaaggggatatagcagcaattgtttttgatgcttttgatatctggggccaagggacaatggtcaccgtctcttca

SEQ ID NO: 98 - protein, 122 aa, Artificial Sequence
  Synthetic
EVQLLESGGGLVQPGGSLRLSCAASGFTFSSYVMNWVRQAPGKGLEWVSAISGSGGSTSYADSVKGRSTISRDNSKNTLYLQMNSLRAEDTAVYYCAKGDIAAIVFDAFDIWGQGTMVTVSS

SEQ ID NO: 99 - DNA, 24 bp, Artificial Sequence
  Synthetic
ggattcacctttagcagctatgtc

SEQ ID NO: 100 - protein, 8 aa, Artificial Sequence
  Synthetic
GFTFSSYV

SEQ ID NO: 101 - DNA, 24 bp, Artificial Sequence
  Synthetic
attagcggaagtggtggtagcaca

SEQ ID NO: 102 - protein, 8 aa, Artificial Sequence
  Synthetic
ISGSGGST

SEQ ID NO: 103 - DNA, 45 bp, Artificial Sequence
  Synthetic
gcgaaaggggatatagcagcaattgtttttgatgcttttgatatc

SEQ ID NO: 104 - protein, 15 aa, Artificial Sequence
  Synthetic
AKGDIAAIVFDAFDI

SEQ ID NO: 105 - DNA, 321 bp, Artificial Sequence
  Synthetic
gacatccagatgacccagtctccatcctccctgtctgcatctgtaggagacagagtcaccatcacttgccaggcgagtcaggacattagcaactgtttaaattggtatcaacacaaaccagggaaagcccctaagctcctgatctacgatgcatcctatttggaaacagggggcccatcaaggttcagtggaagtggatctgggacagattttactttcaccatcagaagcctgcagcctgaagattttgcaacatattactgtcaacagtatgataatctcccgtacacttttggccaggggaccaagctggagatcaaa

SEQ ID NO: 106 - protein, 107 aa, Artificial Sequence
  Synthetic
DIQMTQSPSSLSASVGDRVTITCQASQDISNCLNWYQHKPGKAPKLLIYDASYLETGGPSRFSGSGSGTDFTFTIRSLQPEDFATYYCQQYDNLPYTFGQGTKLEIK

SEQ ID NO: 107 - DNA, 18 bp, Artificial Sequence
  Synthetic
caggacattagcaactgt

SEQ ID NO: 108 - protein, 6 aa, Artificial Sequence
  Synthetic
QDISNC

SEQ ID NO: 109 - DNA, 9 bp, Artificial Sequence
  Synthetic
gatgcatcc

SEQ ID NO: 110 - protein, 3 aa, Artificial Sequence
  Synthetic
DAS

SEQ ID NO: 111 - DNA, 27 bp, Artificial Sequence
  Synthetic
caacagtatgataatctcccgtacact

SEQ ID NO: 112 - protein, 9 aa, Artificial Sequence
  Synthetic
QQYDNLPYT

SEQ ID NO: 113 - DNA, 366 bp, Artificial Sequence
  Synthetic
gaggtgcacttgttggagtctgggggaggcttggtacagccgggggggtccctgagactctcctgtgcagcctctggattcacctttagcagctatgtcatgaactgggtccgccaggctccagggaaggggctggagtgggtctcagctattagcggaagtggtggtagcacatactacggagactccgtgaagggccggtccaccatctccagagacaattccaagaacacactgtatctgcaaatgaaaagcctgagagccgaggacacggccgtatattattgtgcgaaaggggatatagcaccaattgtctttgatgcttttgatatctggggccaagggacaatggtcaccgtctcttca

SEQ ID NO: 114 - protein, 122 aa, Artificial Sequence
  Synthetic
EVHLLESGGGLVQPGGSLRLSCAASGFTFSSYVMNWVRQAPGKGLEWVSAISGSGGSTYYGDSVKGRSTISRDNSKNTLYLQMKSLRAEDTAVYYCAKGDIAPIVFDAFDIWGQGTMVTVSS

SEQ ID NO: 115 - DNA, 24 bp, Artificial Sequence
  Synthetic
ggattcacctttagcagctatgtc

SEQ ID NO: 116 - protein, 8 aa, Artificial Sequence
  Synthetic
GFTFSSYV

SEQ ID NO: 117 - DNA, 24 bp, Artificial Sequence
  Synthetic
attagcggaagtggtggtagcaca

SEQ ID NO: 118 - protein, 8 aa, Artificial Sequence
  Synthetic
ISGSGGST

SEQ ID NO: 119 - DNA, 45 bp, Artificial Sequence
  Synthetic
gcgaaaggggatatagcaccaattgtctttgatgcttttgatatc

SEQ ID NO: 120 - protein, 15 aa, Artificial Sequence
  Synthetic
AKGDIAPIVFDAFDI

SEQ ID NO: 121 - DNA, 321 bp, Artificial Sequence
  Synthetic
gacatccagatgacccagtctccatcctccctgtctgcatctgtaggagacagagtcaccatcacttgccaggcgagtcaggacattagcaactgtttaaattggtatcaacacaaaccagggaaagcccctaaactcctgatctacgatgcatcctatttggaaacaggggtcccatcaaggttcagtggaagtggatctgggacagattttactttcaccatcagcagcctgcagcctgaagatattgcaacatattactgtcaacagtatgataatctcccgtacacttttggccaggggaccaagctggagatcaaa

SEQ ID NO: 122 - protein, 107 aa, Artificial Sequence
  Synthetic
DIQMTQSPSSLSASVGDRVTITCQASQDISNCLNWYQHKPGKAPKLLIYDASYLETGVPSRFSGSGSGTDFTFTISSLQPEDIATYYCQQYDNLPYTFGQGTKLEIK

SEQ ID NO: 123 - DNA, 18 bp, Artificial Sequence
  Synthetic
caggacattagcaactgt

SEQ ID NO: 124 - protein, 6 aa, Artificial Sequence
  Synthetic
QDISNC

SEQ ID NO: 125 - DNA, 9 bp, Artificial Sequence
  Synthetic
gatgcatcc

SEQ ID NO: 126 - protein, 3 aa, Artificial Sequence
  Synthetic
DAS

SEQ ID NO: 127 - DNA, 27 bp, Artificial Sequence
  Synthetic
caacagtatgataatctcccgtacact

SEQ ID NO: 128 - protein, 9 aa, Artificial Sequence
  Synthetic
QQYDNLPYT

SEQ ID NO: 129 - DNA, 372 bp, Artificial Sequence
  Synthetic
caggtgcagctgcaggagtcgggcccaggactggtgaagccttcggagaccctgtccctcacctgcactgtctctggtggctccatcagtaattcctactggagctggatccggcagcccccagggaagggactggagtggattgggtatatctattacagtgggaacaccaactacaacccctccctcaagagtcgagtcaccatatcagtggacacgtccaagaaccagttctccctgaagctgagctctgtgaccgccgcagacacggccgtgtattactgtgcgagagtcaatgactacagtaattatgactcctactattacggtatggacgtctggggccaagggaccacggtcaccgtctcctca

SEQ ID NO: 130 - protein, 124 aa, Artificial Sequence
  Synthetic
QVQLQESGPGLVKPSETLSLTCTVSGGSISNSYWSWIRQPPGKGLEWIGYIYYSGNTNYNPSLKSRVTISVDTSKNQFSLKLSSVTAADTAVYYCARVNDYSNYDSYYYGMDVWGQGTTVTVSS

SEQ ID NO: 131 - DNA, 24 bp, Artificial Sequence
  Synthetic
ggtggctccatcagtaattcctac

SEQ ID NO: 132 - protein, 8 aa, Artificial Sequence
  Synthetic
GGSISNSY

SEQ ID NO: 133 - DNA, 21 bp, Artificial Sequence
  Synthetic
atctattacagtgggaacacc

SEQ ID NO: 134 - protein, 7 aa, Artificial Sequence
  Synthetic
IYYSGNT

SEQ ID NO: 135 - DNA, 54 bp, Artificial Sequence
  Synthetic
gcgagagtcaatgactacagtaattatgactcctactattacggtatggacgtc

SEQ ID NO: 136 - protein, 18 aa, Artificial Sequence
  Synthetic
ARVNDYSNYDSYYYGMDV

SEQ ID NO: 137 - DNA, 321 bp, Artificial Sequence
  Synthetic
gccatccagatgacccagtctccatcctccctgtctgcatctgtgggagacagagtcaccatcacttgccgggcaagtcagggcattagaaatgatttaggctggtatcagcagaaaccagggaaagcccctaaactcctgatctatgctgcatccagtttacaaagtggggtcccatcaaggttcagcggcagtggatctggcacagatttcactctcaccatcagcagcctgcagcctgaagattttgcaacttattactgtctacaagattacaattaccctccgacgttcggccaagggaccaaggtggacatcaag

SEQ ID NO: 138 - protein, 107 aa, Artificial Sequence
  Synthetic
AIQMTQSPSSLSASVGDRVTITCRASQGIRNDLGWYQQKPGKAPKLLIYAASSLQSGVPSRFSGSGSGTDFTLTISSLQPEDFATYYCLQDYNYPPTFGQGTKVDIK

SEQ ID NO: 139 - DNA, 18 bp, Artificial Sequence
  Synthetic
cagggcattagaaatgat

SEQ ID NO: 140 - protein, 6 aa, Artificial Sequence
  Synthetic
QGIRND

SEQ ID NO: 141 - DNA, 9 bp, Artificial Sequence
  Synthetic
gctgcatcc

SEQ ID NO: 142 - protein, 3 aa, Artificial Sequence
  Synthetic
AAS

SEQ ID NO: 143 - DNA, 27 bp, Artificial Sequence
  Synthetic
ctacaagattacaattaccctccgacg

SEQ ID NO: 144 - protein, 9 aa, Artificial Sequence
  Synthetic
LQDYNYPPT

SEQ ID NO: 145 - DNA, 360 bp, Artificial Sequence
  Synthetic
caggtgcagctggtggagtctgggggaggcgtggtccagcctgggaggtccctgagactcacctgtgcagcgtctggattcaccttcagtagctttggcatgcactgggtccgacaggctccaggcaaggggctggagtgggtggcaattatatggtatgatggaagtaataaatactatgcagattccgtgaagggccgattcaccatctccagagacaattccaagaacacgctgtatctgcaaatgaacagcctgcgagccgaggacacggctgtgtattactgtgcgagagaggataactggacccgggattactttgactactggggccagggaaccctggtcaccgtctcctca

SEQ ID NO: 146 - protein, 120 aa, Artificial Sequence
  Synthetic
QVQLVESGGGVVQPGRSLRLTCAASGFTFSSFGMHWVRQAPGKGLEWVAIIWYDGSNKYYADSVKGRFTISRDNSKNTLYLQMNSLRAEDTAVYYCAREDNWTRDYFDYWGQGTLVTVSS

SEQ ID NO: 147 - DNA, 24 bp, Artificial Sequence
  Synthetic
ggattcaccttcagtagctttggc

SEQ ID NO: 148 - protein, 8 aa, Artificial Sequence
  Synthetic
GFTFSSFG

SEQ ID NO: 149 - DNA, 24 bp, Artificial Sequence
  Synthetic
atatggtatgatggaagtaataaa

SEQ ID NO: 150 - protein, 8 aa, Artificial Sequence
  Synthetic
IWYDGSNK

SEQ ID NO: 151 - DNA, 39 bp, Artificial Sequence
  Synthetic
gcgagagaggataactggacccgggattactttgactac

SEQ ID NO: 152 - protein, 13 aa, Artificial Sequence
  Synthetic
AREDNWTRDYFDY

SEQ ID NO: 153 - DNA, 324 bp, Artificial Sequence
  Synthetic
gaaattgtgttgacacagtctccagccaccctgtctttgtctccaggggaaagagccaccctctcctgcagggccagtcagagtgttagcaacttcttagcctggtatcaacagaagcctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccgctccctttcggcggagggaccaaggtggagatcaaa

SEQ ID NO: 154 - protein, 108 aa, Artificial Sequence
  Synthetic
EIVLTQSPATLSLSPGERATLSCRASQSVSNFLAWYQQKPGQAPRLLIYDASNRATGIPARFSGSGSGTDFTLTISSLEPEDFAVYYCQQRSNWPPLPFGGGTKVEIK

SEQ ID NO: 155 - DNA, 18 bp, Artificial Sequence
  Synthetic
cagagtgttagcaacttc

SEQ ID NO: 156 - protein, 6 aa, Artificial Sequence
  Synthetic
QSVSNF

SEQ ID NO: 157 - DNA, 9 bp, Artificial Sequence
  Synthetic
gatgcatcc

SEQ ID NO: 158 - protein, 3 aa, Artificial Sequence
  Synthetic
DAS

SEQ ID NO: 159 - DNA, 30 bp, Artificial Sequence
  Synthetic
cagcagcgtagcaactggcctccgctccct

SEQ ID NO: 160 - protein, 10 aa, Artificial Sequence
  Synthetic
QQRSNWPPLP

SEQ ID NO: 161 - DNA, 357 bp, Artificial Sequence
  Synthetic
gaggtacagatggtggagtctgggggaggcttggtccagcctggggggtccctgagactctcctgtgcagcctctagattcacccttagtaactattggatgggctgggtccgccaggctccagggaaggggctggagtgggtggccaacataaagcaagatgggagtgagaaatactatgtggactctgtgaggggccgattcaccatctccagagacaacgccaagaactctctatatctgcaaatgaacagcctgagagccgaggacacggctgtgtattactgtgcgagggattacgatttttggaggtcctttgactactggggccagggaaccctggtcaccgtcccctca

SEQ ID NO: 162 - protein, 119 aa, Artificial Sequence
  Synthetic
EVQMVESGGGLVQPGGSLRLSCAASRFTLSNYWMGWVRQAPGKGLEWVANIKQDGSEKYYVDSVRGRFTISRDNAKNSLYLQMNSLRAEDTAVYYCARDYDFWRSFDYWGQGTLVTVPS

SEQ ID NO: 163 - DNA, 24 bp, Artificial Sequence
  Synthetic
agattcacccttagtaactattgg

SEQ ID NO: 164 - protein, 8 aa, Artificial Sequence
  Synthetic
RFTLSNYW

SEQ ID NO: 165 - DNA, 24 bp, Artificial Sequence
  Synthetic
ataaagcaagatgggagtgagaaa

SEQ ID NO: 166 - protein, 8 aa, Artificial Sequence
  Synthetic
IKQDGSEK

SEQ ID NO: 167 - DNA, 36 bp, Artificial Sequence
  Synthetic
gcgagggattacgatttttggaggtcctttgactac

SEQ ID NO: 168 - protein, 12 aa, Artificial Sequence
  Synthetic
ARDYDFWRSFDY

SEQ ID NO: 169 - DNA, 322 bp, Artificial Sequence
  Synthetic
gacatccagatgacccagtctccatcttccgtgtctgcatctgtaggagacagagtcaccatcacctgtcgggcgagtcagggtgttagcagctggttagcctggtatcagcagacaccagggaaagcccctaagctcctgatctatgttgtatcaagtttgcaaagtggggtcccatcaagattcagcggcagtggatctgggacagatttcactctcaccatcaacagcctgcagcctgaagattttgcaacttactattgtcaacagggtaacagtttcccgtacacttttggccaggggaccaagctggagatcaaak

SEQ ID NO: 170 - protein, 107 aa, Artificial Sequence
  Synthetic
DIQMTQSPSSVSASVGDRVTITCRASQGVSSWLAWYQQTPGKAPKLLIYVVSSLQSGVPSRFSGSGSGTDFTLTINSLQPEDFATYYCQQGNSFPYTFGQGTKLEIK

SEQ ID NO: 171 - DNA, 18 bp, Artificial Sequence
  Synthetic
cagggtgttagcagctgg

SEQ ID NO: 172 - protein, 6 aa, Artificial Sequence
  Synthetic
QGVSSW

SEQ ID NO: 173 - DNA, 9 bp, Artificial Sequence
  Synthetic
gttgtatca

SEQ ID NO: 174 - protein, 3 aa, Artificial Sequence
  Synthetic
VVS

SEQ ID NO: 175 - DNA, 27 bp, Artificial Sequence
  Synthetic
caacagggtaacagtttcccgtacact

SEQ ID NO: 176 - protein, 9 aa, Artificial Sequence
  Synthetic
QQGNSFPYT

SEQ ID NO: 177 - DNA, 357 bp, Artificial Sequence
  Synthetic
caggtgcagctggtggagtctgggggaggcgtggtccagcctgggaggtccctgagactctcctgtgcagcgtctggattcaccttcagtagctatggcatacactgggtccgccaggctccaggcaaggggctggagtgggtggcaattctatggtatgatggaagtaataaatactatgccgactccgtgaagggccgattcaccatctccagagacaattccaaaaccacgctgtatctgcaaatgaacagcctgagagccgaggacacggctgtgtattactgtgcgagagaaaactataacgacttgaacttcgatctctggggccgtggcaccctggtcactgtctcctca

SEQ ID NO: 178 - protein, 119 aa, Artificial Sequence
  Synthetic
QVQLVESGGGVVQPGRSLRLSCAASGFTFSSYGIHWVRQAPGKGLEWVAILWYDGSNKYYADSVKGRFTISRDNSKTTLYLQMNSLRAEDTAVYYCARENYNDLNFDLWGRGTLVTVSS

SEQ ID NO: 179 - DNA, 24 bp, Artificial Sequence
  Synthetic
ggattcaccttcagtagctatggc

SEQ ID NO: 180 - protein, 8 aa, Artificial Sequence
  Synthetic
GFTFSSYG

SEQ ID NO: 181 - DNA, 24 bp, Artificial Sequence
  Synthetic
ctatggtatgatggaagtaataaa

SEQ ID NO: 182 - protein, 8 aa, Artificial Sequence
  Synthetic
LWYDGSNK

SEQ ID NO: 183 - DNA, 36 bp, Artificial Sequence
  Synthetic
gcgagagaaaactataacgacttgaacttcgatctc

SEQ ID NO: 184 - protein, 12 aa, Artificial Sequence
  Synthetic
ARENYNDLNFDL

SEQ ID NO: 185 - DNA, 324 bp, Artificial Sequence
  Synthetic
gacatccagttgacccagtctccatccttcctgtctgcatctgtaggagacagagtcaccatcacttgctgggccagtcagggcattagcagttatttagcctggtatcagcacaaaccagggaaagcccctaagctcctgatctatgctgcatccactttgcaaagtggggtcccatcacggttcagcggcagtggatctgggatagaattcactctcacaatcagcagcctgcagcctgaagattttgcaacttattactgtcaacagcttaaaagttaccctccgtggacgttcggccaagggaccaaggtggaaatcaga

SEQ ID NO: 186 - protein, 108 aa, Artificial Sequence
  Synthetic
DIQLTQSPSFLSASVGDRVTITCWASQGISSYLAWYQHKPGKAPKLLIYAASTLQSGVPSRFSGSGSGIEFTLTISSLQPEDFATYYCQQLKSYPPWTFGQGTKVEIR

SEQ ID NO: 187 - DNA, 18 bp, Artificial Sequence
  Synthetic
cagggcattagcagttat

SEQ ID NO: 188 - protein, 6 aa, Artificial Sequence
  Synthetic
QGISSY

SEQ ID NO: 189 - DNA, 9 bp, Artificial Sequence
  Synthetic
gctgcatcc

SEQ ID NO: 190 - protein, 3 aa, Artificial Sequence
  Synthetic
AAS

SEQ ID NO: 191 - DNA, 30 bp, Artificial Sequence
  Synthetic
caacagcttaaaagttaccctccgtggacg

SEQ ID NO: 192 - protein, 10 aa, Artificial Sequence
  Synthetic
QQLKSYPPWT

SEQ ID NO: 193 - DNA, 348 bp, Artificial Sequence
  Synthetic
gaggtgcagctggtggagtctggaggaggcttggtccagcctggggggtccctgagactctcatgtgcagcctctggtttcaccgtcagtagcaactacatgagctgggtccgccaggctccagggaaggggctggagtgggtctcagttatttatagcggtggtaacacatactacgcagactccgtgaagggccgattcaccatctccagacacaattccaagaacacgctgtatcttcaaatgaacagcctgagagctgaggacacggccgtgtactactgtgcgcgagatctaggcattaagtctgactattggggccagggaaccctggtcaccgtctcctca

SEQ ID NO: 194 - protein, 116 aa, Artificial Sequence
  Synthetic
EVQLVESGGGLVQPGGSLRLSCAASGFTVSSNYMSWVRQAPGKGLEWVSVIYSGGNTYYADSVKGRFTISRHNSKNTLYLQMNSLRAEDTAVYYCARDLGIKSDYWGQGTLVTVSS

SEQ ID NO: 195 - DNA, 24 bp, Artificial Sequence
  Synthetic
ggtttcaccgtcagtagcaactac

SEQ ID NO: 196 - protein, 8 aa, Artificial Sequence
  Synthetic
GFTVSSNY

SEQ ID NO: 197 - DNA, 21 bp, Artificial Sequence
  Synthetic
atttatagcggtggtaacaca

SEQ ID NO: 198 - protein, 7 aa, Artificial Sequence
  Synthetic
IYSGGNT

SEQ ID NO: 199 - DNA, 30 bp, Artificial Sequence
  Synthetic
gcgcgagatctaggcattaagtctgactat

SEQ ID NO: 200 - protein, 10 aa, Artificial Sequence
  Synthetic
ARDLGIKSDY

SEQ ID NO: 201 - DNA, 321 bp, Artificial Sequence
  Synthetic
gaaattgtgctgactcagtctccagactttcagtctgtgactccaaaggagaaagtcaccatcacctgccgggccagtcagagcattggtactaccttacactggtaccagcagaaaccagatcagtctccaaaactcctcatcaagtatgtttcccagtccctctcaggggtcccctcgaggttcagtggcagtggatctgggacagatttcaccctcaccatcaatagcctggaagctgaagatgctgcaacgtattactgtcatcagagtagtagtttaccgtggacgttcggccaagggaccaaggtggaaatcaaa

SEQ ID NO: 202 - protein, 107 aa, Artificial Sequence
  Synthetic
EIVLTQSPDFQSVTPKEKVTITCRASQSIGTTLHWYQQKPDQSPKLLIKYVSQSLSGVPSRFSGSGSGTDFTLTINSLEAEDAATYYCHQSSSLPWTFGQGTKVEIK

SEQ ID NO: 203 - DNA, 18 bp, Artificial Sequence
  Synthetic
cagagcattggtactacc

SEQ ID NO: 204 - protein, 6 aa, Artificial Sequence
  Synthetic
QSIGTT

SEQ ID NO: 205 - DNA, 9 bp, Artificial Sequence
  Synthetic
tatgtttcc

SEQ ID NO: 206 - protein, 3 aa, Artificial Sequence
  Synthetic
YVS

SEQ ID NO: 207 - DNA, 27 bp, Artificial Sequence
  Synthetic
catcagagtagtagtttaccgtggacg

SEQ ID NO: 208 - protein, 9 aa, Artificial Sequence
  Synthetic
HQSSSLPWT

SEQ ID NO: 209 - DNA, 360 bp, Artificial Sequence
  Synthetic
caggtgcagctggtggagtctgggggaggcgtggtccagcctgggaggtccctgagactctcctgtgcagcctctggattcaccttcactaactatggcatgcactgggtccgccaggctccaggcaaggggctggagtgggtggcagctatatcatatgatggaactaataaatactatgcagactccgtgaagggccgattcaccatctccagagacgattccaagaacacgctgtgtctgcaaatgaacagcctgagagctgaggacacggctgtgtattactgtgcgaaagaggggactggtgaaggtttctcctttgactactggggccagggaaccctggtcaccgtctcctca

SEQ ID NO: 210 - protein, 120 aa, Artificial Sequence
  Synthetic
QVQLVESGGGVVQPGRSLRLSCAASGFTFTNYGMHWVRQAPGKGLEWVAAISYDGTNKYYADSVKGRFTISRDDSKNTLCLQMNSLRAEDTAVYYCAKEGTGEGFSFDYWGQGTLVTVSS

SEQ ID NO: 211 - DNA, 24 bp, Artificial Sequence
  Synthetic
ggattcaccttcactaactatggc

SEQ ID NO: 212 - protein, 8 aa, Artificial Sequence
  Synthetic
GFTFTNYG

SEQ ID NO: 213 - DNA, 24 bp, Artificial Sequence
  Synthetic
atatcatatgatggaactaataaa

SEQ ID NO: 214 - protein, 8 aa, Artificial Sequence
  Synthetic
ISYDGTNK

SEQ ID NO: 215 - DNA, 39 bp, Artificial Sequence
  Synthetic
gcgaaagaggggactggtgaaggtttctcctttgactac

SEQ ID NO: 216 - protein, 13 aa, Artificial Sequence
  Synthetic
AKEGTGEGFSFDY

SEQ ID NO: 217 - DNA, 336 bp, Artificial Sequence
  Synthetic
gatattgtgatgactcagtctccactctccctgcccgtcacccctggagagccggcctccatctcctgcaggtctagtcagagcctcctacattttaatggatacaactatttggattggtacctgcagaagccagggcagtctccacagctcctgatctatttgggttctaatcgggcctccggggtccctgacaggttcagtggcagtggatcaggcacagattttacactgaaagtcagcagagtggaggctgaggatgttggggtttattactgcatgcaagctctacaaactccattcactttcggccctgggaccaaagtggatatcaaa

SEQ ID NO: 218 - protein, 112 aa, Artificial Sequence
  Synthetic
DIVMTQSPLSLPVTPGEPASISCRSSQSLLHFNGYNYLDWYLQKPGQSPQLLIYLGSNRASGVPDRFSGSGSGTDFTLKVSRVEAEDVGVYYCMQALQTPFTFGPGTKVDIK

SEQ ID NO: 219 - DNA, 33 bp, Artificial Sequence
  Synthetic
cagagcctcctacattttaatggatacaactat

SEQ ID NO: 220 - protein, 11 aa, Artificial Sequence
  Synthetic
QSLLHFNGYNY

SEQ ID NO: 221 - DNA, 9 bp, Artificial Sequence
  Synthetic
ttgggttct

SEQ ID NO: 222 - protein, 3 aa, Artificial Sequence
  Synthetic
LGS

SEQ ID NO: 223 - DNA, 27 bp, Artificial Sequence
  Synthetic
atgcaagctctacaaactccattcact

SEQ ID NO: 224 - protein, 9 aa, Artificial Sequence
  Synthetic
MQALQTPFT

SEQ ID NO: 225 - DNA, 360 bp, Artificial Sequence
  Synthetic
gaggtgcagctggtggagtctgggggaggcttggtacagcctggggggtccctgagactctcctgtgcagcctctggattcaccttcagtatctacgacatgcactgggtccgccaagctacaggaaaaggtctggagtgggtctcaggtattggtaatgctggtgacacatactatgcaggctccgtgaagggccgattcaccatctccagagaaaatgccaagaactccttgtatcttcaaatgaacagcctgagagccggggacacggctgtatattactgtgcaagagagggtcccaactactactactatggtatggacgtctggggccaagggaccacggtcaccgtctcctca

SEQ ID NO: 226 - protein, 120 aa, Artificial Sequence
  Synthetic
EVQLVESGGGLVQPGGSLRLSCAASGFTFSIYDMHWVRQATGKGLEWVSGIGNAGDTYYAGSVKGRFTISRENAKNSLYLQMNSLRAGDTAVYYCAREGPNYYYYGMDVWGQGTTVTVSS

SEQ ID NO: 227 - DNA, 24 bp, Artificial Sequence
  Synthetic
ggattcaccttcagtatctacgac

SEQ ID NO: 228 - protein, 8 aa, Artificial Sequence
  Synthetic
GFTFSIYD

SEQ ID NO: 229 - DNA, 21 bp, Artificial Sequence
  Synthetic
attggtaatgctggtgacaca

SEQ ID NO: 230 - protein, 7 aa, Artificial Sequence
  Synthetic
IGNAGDT

SEQ ID NO: 231 - DNA, 42 bp, Artificial Sequence
  Synthetic
gcaagagagggtcccaactactactactatggtatggacgtc

SEQ ID NO: 232 - protein, 14 aa, Artificial Sequence
  Synthetic
AREGPNYYYYGMDV

SEQ ID NO: 233 - DNA, 321 bp, Artificial Sequence
  Synthetic
gccatccagatgacccagtctccatcctccctgtctgcatctgtaggagacagagtcaccatcacttgccgggcaagtcagggcattagagatgatttaggctggtatcagcagaaaccagggaaagcccctaagctcctgatctatgctgcatccagtttacaaagtggggtcccatcaaggttcagcggcagtggatctggcacagatttcactctcaccatcagcagcctgcagcctgaagattttgcaacttattactgtctacaagattacaattacccgtggacgttcggccaagggaccaaggtggagatcaaa

SEQ ID NO: 234 - protein, 107 aa, Artificial Sequence
  Synthetic
AIQMTQSPSSLSASVGDRVTITCRASQGIRDDLGWYQQKPGKAPKLLIYAASSLQSGVPSRFSGSGSGTDFTLTISSLQPEDFATYYCLQDYNYPWTFGQGTKVEIK

SEQ ID NO: 235 - DNA, 18 bp, Artificial Sequence
  Synthetic
cagggcattagagatgat

SEQ ID NO: 236 - protein, 6 aa, Artificial Sequence
  Synthetic
QGIRDD

SEQ ID NO: 237 - DNA, 9 bp, Artificial Sequence
  Synthetic
gctgcatcc

SEQ ID NO: 238 - protein, 3 aa, Artificial Sequence
  Synthetic
AAS

SEQ ID NO: 239 - DNA, 27 bp, Artificial Sequence
  Synthetic
ctacaagattacaattacccgtggacg

SEQ ID NO: 240 - protein, 9 aa, Artificial Sequence
  Synthetic
LQDYNYPWT

SEQ ID NO: 241 - DNA, 351 bp, Artificial Sequence
  Synthetic
caggtgcagctggtgcagtctggggctgaggtgaagaagcctggggcctcagtgaaggtctcctgcaaggcttctggattcaccttcaccagttatgatatcaactgggtgcgacaggccactggacaggggcttgagtggatgggatggatgaaccctaagagtggtaacacagactatgcacaaaagttcctgggcagagtcaccctgaccaggaacacctccaaaagcacagcctacatggagctgagcagcctgagatctgaggacacggccgtgtactactgtgcgagaggaaagcagctcgtctttgactactggggccagggaaccctggtcaccgtctccgca

SEQ ID NO: 242 - protein, 117 aa, Artificial Sequence
  Synthetic
QVQLVQSGAEVKKPGASVKVSCKASGFTFTSYDINWVRQATGQGLEWMGWMNPKSGNTDYAQKFLGRVTLTRNTSKSTAYMELSSLRSEDTAVYYCARGKQLVFDYWGQGTLVTVSA

SEQ ID NO: 243 - DNA, 24 bp, Artificial Sequence
  Synthetic
ggattcaccttcaccagttatgat

SEQ ID NO: 244 - protein, 8 aa, Artificial Sequence
  Synthetic
GFTFTSYD

SEQ ID NO: 245 - DNA, 24 bp, Artificial Sequence
  Synthetic
atgaaccctaagagtggtaacaca

SEQ ID NO: 246 - protein, 8 aa, Artificial Sequence
  Synthetic
MNPKSGNT

SEQ ID NO: 247 - DNA, 30 bp, Artificial Sequence
  Synthetic
gcgagaggaaagcagctcgtctttgactac

SEQ ID NO: 248 - protein, 10 aa, Artificial Sequence
  Synthetic
ARGKQLVFDY

SEQ ID NO: 249 - DNA, 321 bp, Artificial Sequence
  Synthetic
gacatccagatgacccagtctccatcctccctgtctgcatctgtaggagacagagtcaccatcacttgccaggcgaatcaggacattactaactatttaaattggtatcagaagaaaccagggaaagcccctaagctcctgatctacgatgcatccaatttggaaacaggggtcccatcaaggttcagtggaagtggatatgggacagattttactttcaccatcagcagcctgcagcctgaagatattgcaacatattactgtcaacagtatgataatctcccattcactttcggccctgggaccaaagtggatatcaaa

SEQ ID NO: 250 - protein, 107 aa, Artificial Sequence
  Synthetic
DIQMTQSPSSLSASVGDRVTITCQANQDITNYLNWYQKKPGKAPKLLIYDASNLETGVPSRFSGSGYGTDFTFTISSLQPEDIATYYCQQYDNLPFTFGPGTKVDIK

SEQ ID NO: 251 - DNA, 18 bp, Artificial Sequence
  Synthetic
caggacattactaactat

SEQ ID NO: 252 - protein, 6 aa, Artificial Sequence
  Synthetic
QDITNY

SEQ ID NO: 253 - DNA, 9 bp, Artificial Sequence
  Synthetic
gatgcatcc

SEQ ID NO: 254 - protein, 3 aa, Artificial Sequence
  Synthetic
DAS

SEQ ID NO: 255 - DNA, 27 bp, Artificial Sequence
  Synthetic
caacagtatgataatctcccattcact

SEQ ID NO: 256 - protein, 9 aa, Artificial Sequence
  Synthetic
QQYDNLPFT

SEQ ID NO: 257 - DNA, 348 bp, Artificial Sequence
  Synthetic
gaggtgcagctggtggagtctgggggaggcctggtcaagcctggggggtccctgagactctcctgtgcagcctctggattcaccttcagttactatagcatgatctgggtccgccaggctccagggaaggggctggagtgggtctcatccatcagtagtagtagtagttacatatactacgcagactcagtgaagggccgattcaccatctccagagacaacgccaagaaatcaatgtatctgcaaatgaacagcctgagagccgaggacacggctgtgtattactgtgcgagaggtagtggctaccctgactactggggccagggaaccctggtcaccgtctcctca

SEQ ID NO: 258 - protein, 116 aa, Artificial Sequence
  Synthetic
EVQLVESGGGLVKPGGSLRLSCAASGFTFSYYSMIWVRQAPGKGLEWVSSISSSSSYIYYADSVKGRFTISRDNAKKSMYLQMNSLRAEDTAVYYCARGSGYPDYWGQGTLVTVSS

SEQ ID NO: 259 - DNA, 24 bp, Artificial Sequence
  Synthetic
ggattcaccttcagttactatagc

SEQ ID NO: 260 - protein, 8 aa, Artificial Sequence
  Synthetic
GFTFSYYS

SEQ ID NO: 261 - DNA, 24 bp, Artificial Sequence
  Synthetic
atcagtagtagtagtagttacata

SEQ ID NO: 262 - protein, 8 aa, Artificial Sequence
  Synthetic
ISSSSSYI

SEQ ID NO: 263 - DNA, 27 bp, Artificial Sequence
  Synthetic
gcgagaggtagtggctaccctgactac

SEQ ID NO: 264 - protein, 9 aa, Artificial Sequence
  Synthetic
ARGSGYPDY

SEQ ID NO: 265 - DNA, 321 bp, Artificial Sequence
  Synthetic
gacatccagatgacccagtctccatcctccctgtctgcatctgtaggagacagagtcaccatcacttgccgggcgagtcagggcattaacaattatttagcctggtatcagcagaaaccagggaaagttcctaagctcctgatctatgctgcatccactttacgatcaggggtcccatctcggttcagtggcagtggatctgggacagatttcactctcaccatcagcagcctgcagcctgaagatgttgcaacttattactgtcaaaagtataacagtgccccattcactttcggccctgggaccaaagtggatatcaaa

SEQ ID NO: 266 - protein, 107 aa, Artificial Sequence
  Synthetic
DIQMTQSPSSLSASVGDRVTITCRASQGINNYLAWYQQKPGKVPKLLIYAASTLRSGVPSRFSGSGSGTDFTLTISSLQPEDVATYYCQKYNSAPFTFGPGTKVDIK

SEQ ID NO: 267 - DNA, 18 bp, Artificial Sequence
  Synthetic
cagggcattaacaattat

SEQ ID NO: 268 - protein, 6 aa, Artificial Sequence
  Synthetic
QGINNY

SEQ ID NO: 269 - DNA, 9 bp, Artificial Sequence
  Synthetic
gctgcatcc

SEQ ID NO: 270 - protein, 3 aa, Artificial Sequence
  Synthetic
AAS

SEQ ID NO: 271 - DNA, 27 bp, Artificial Sequence
  Synthetic
caaaagtataacagtgccccattcact

SEQ ID NO: 272 - protein, 9 aa, Artificial Sequence
  Synthetic
QKYNSAPFT

SEQ ID NO: 273 - DNA, 357 bp, Artificial Sequence
  Synthetic
gaggtacagatggtggagtctgggggaggcttggtccagcctggggggtccctgagactctcctgtgcagcctctggattcacccttagtaactattggatgggctgggtccgccaggctccagggaaggggctggagtgggtggccaacataaagcaagatgggagtgagaaatactatgtggactctgtgaggggccgattcaccatctccagagacaacgccaagaactcactgtatctgcaaatgaacagcctgagagccgaggacacggctgtttattactgtgcgagggattacgatttttggaggtcctttgactactggggccagggaaccctggtcaccgtctcctca

SEQ ID NO: 274 - protein, 119 aa, Artificial Sequence
  Synthetic
EVQMVESGGGLVQPGGSLRLSCAASGFTLSNYWMGWVRQAPGKGLEWVANIKQDGSEKYYVDSVRGRFTISRDNAKNSLYLQMNSLRAEDTAVYYCARDYDFWRSFDYWGQGTLVTVSS

SEQ ID NO: 275 - DNA, 24 bp, Artificial Sequence
  Synthetic
ggattcacccttagtaactattgg

SEQ ID NO: 276 - protein, 8 aa, Artificial Sequence
  Synthetic
GFTLSNYW

SEQ ID NO: 277 - DNA, 24 bp, Artificial Sequence
  Synthetic
ataaagcaagatgggagtgagaaa

SEQ ID NO: 278 - protein, 8 aa, Artificial Sequence
  Synthetic
IKQDGSEK

SEQ ID NO: 279 - DNA, 36 bp, Artificial Sequence; identical to SEQ ID NO: 167
  Synthetic

SEQ ID NO: 280 - protein, 12 aa, Artificial Sequence
  Synthetic
ARDYDFWRSFDY

SEQ ID NO: 281 - DNA, 321 bp, Artificial Sequence
  Synthetic
gacatccagatgacccagtctccatcttccgtgtctgcatctgtaggagacagagtcaccatcacctgtcgggcgagtcagggtgttagcagctggttagcctggtatcagcagaaaccagggaaaacccctaagctcctgatctatgttgtatccagtttgcaaagtggggtcccatcaaggttcagcggccgtggatctgggacagatttcactctcaccatcaacagcctgcagcctgaagattttgcaacttactattgtcaacagggtaacagtttcccgtacacttttggccaggggaccaagctggagatcaaa

SEQ ID NO: 282 - protein, 107 aa, Artificial Sequence
  Synthetic
DIQMTQSPSSVSASVGDRVTITCRASQGVSSWLAWYQQKPGKTPKLLIYVVSSLQSGVPSRFSGRGSGTDFTLTINSLQPEDFATYYCQQGNSFPYTFGQGTKLEIK

SEQ ID NO: 283 - DNA, 18 bp, Artificial Sequence
  Synthetic
cagggtgttagcagctgg

SEQ ID NO: 284 - protein, 6 aa, Artificial Sequence
  Synthetic
QGVSSW

SEQ ID NO: 285 - DNA, 9 bp, Artificial Sequence
  Synthetic
gttgtatcc

SEQ ID NO: 286 - protein, 3 aa, Artificial Sequence
  Synthetic
VVS

SEQ ID NO: 287 - DNA, 27 bp, Artificial Sequence
  Synthetic
caacagggtaacagtttcccgtacact

SEQ ID NO: 288 - protein, 9 aa, Artificial Sequence
  Synthetic
QQGNSFPYT

SEQ ID NO: 289 - DNA, 366 bp, Artificial Sequence
  Synthetic
gaagtgcagctggtggagtctgggggaggcttggtacagactggcaggtccctgagactctcctgtgcagcctctggattcacgtttgatgattatgccatgaactgggtccggcaagctccagggaagggcctggagtgggtctcaggtattagttggaatagtggtaacataggctatgcggactctgtgaagggccgattcaccatctccagagacaacgccaagaattccctgtatctgcaaatgaacagtctgagagctgaggacacggccttgtattactgtgtaaaatatatagggcagcagctggtacaggactactttgactactggggccagggaaccctggtcaccgtctcctca

SEQ ID NO: 290 - protein, 122 aa, Artificial Sequence
  Synthetic
EVQLVESGGGLVQTGRSLRLSCAASGFTFDDYAMNWVRQAPGKGLEWVSGISWNSGNIGYADSVKGRFTISRDNAKNSLYLQMNSLRAEDTALYYCVKYIGQQLVQDYFDYWGQGTLVTVSS

SEQ ID NO: 291 - DNA, 24 bp, Artificial Sequence
  Synthetic
ggattcacgtttgatgattatgcc

SEQ ID NO: 292 - protein, 8 aa, Artificial Sequence
  Synthetic
GFTFDDYA

SEQ ID NO: 293 - DNA, 24 bp, Artificial Sequence
  Synthetic
attagttggaatagtggtaacata

SEQ ID NO: 294 - protein, 8 aa, Artificial Sequence
  Synthetic
ISWNSGNI

SEQ ID NO: 295 - DNA, 45 bp, Artificial Sequence
  Synthetic
gtaaaatatatagggcagcagctggtacaggactactttgactac

SEQ ID NO: 296 - protein, 15 aa, Artificial Sequence
  Synthetic
VKYIGQQLVQDYFDY

SEQ ID NO: 297 - DNA, 321 bp, Artificial Sequence
  Synthetic
gacatccagatgacccagtctccatcctccctgtctgcatctgtaggagacagagtcaccatcacttgccaggcgagtcaggacattaccaattatttaaattggtatcagcagagaccagggaaagcccctaagctcctgatctacgatgcattcaatttggaaagaggggtcccatcaaggttcagtggaagtggatatgggacatattttactttcaccatcagcagcctgcagcctgaagatattgcaatatattactgtcaacagtatgataatctcccgctcactttcggcggagggaccaaggtggagatcgaa

SEQ ID NO: 298 - protein, 107 aa, Artificial Sequence
  Synthetic
DIQMTQSPSSLSASVGDRVTITCQASQDITNYLNWYQQRPGKAPKLLIYDAFNLERGVPSRFSGSGYGTYFTFTISSLQPEDIAIYYCQQYDNLPLTFGGGTKVEIE

SEQ ID NO: 299 - DNA, 18 bp, Artificial Sequence
  Synthetic
caggacattaccaattat

SEQ ID NO: 300 - protein, 6 aa, Artificial Sequence
  Synthetic
QDITNY

SEQ ID NO: 301 - DNA, 9 bp, Artificial Sequence
  Synthetic
gatgcattc

SEQ ID NO: 302 - protein, 3 aa, Artificial Sequence
  Synthetic
DAF

SEQ ID NO: 303 - DNA, 27 bp, Artificial Sequence
  Synthetic
caacagtatgataatctcccgctcact

SEQ ID NO: 304 - protein, 9 aa, Artificial Sequence
  Synthetic
QQYDNLPLT

SEQ ID NO: 305 - DNA, 357 bp, Artificial Sequence
  Synthetic
caggtgcagctggtggagtctgggggaggcgtggtccagcctgggaggtccctgagactctcctgtgcagcgtctggattcaccttcagtagtcatggcatgcattgggtccgccaggctccaggcaaggggctggagtgggtggcagtcatatggtatgatggaagtaataaataccatgcagactccgtgaagggccgattcaccatctacagagacaattccaagaacacgctggatctgcaaatgaacagcctgagagtcgaggacacggctatgtattactgtgcgagagaagacagtaataacgaagatcttgactattggggccagggaaccctggtcaccgtttcctca

SEQ ID NO: 306 - protein, 119 aa, Artificial Sequence
  Synthetic
QVQLVESGGGVVQPGRSLRLSCAASGFTFSSHGMHWVRQAPGKGLEWVAVIWYDGSNKYHADSVKGRFTIYRDNSKNTLDLQMNSLRVEDTAMYYCAREDSNNEDLDYWGQGTLVTVSS

SEQ ID NO: 307 - DNA, 24 bp, Artificial Sequence
  Synthetic
ggattcaccttcagtagtcatggc

SEQ ID NO: 308 - protein, 8 aa, Artificial Sequence
  Synthetic
GFTFSSHG

SEQ ID NO: 309 - DNA, 24 bp, Artificial Sequence
  Synthetic
atatggtatgatggaagtaataaa

SEQ ID NO: 310 - protein, 8 aa, Artificial Sequence
  Synthetic
IWYDGSNK

SEQ ID NO: 311 - DNA, 36 bp, Artificial Sequence
  Synthetic
gcgagagaagacagtaataacgaagatcttgactat

SEQ ID NO: 312 - protein, 12 aa, Artificial Sequence
  Synthetic
AREDSNNEDLDY

SEQ ID NO: 313 - DNA, 324 bp, Artificial Sequence
  Synthetic
gacatccagatgacccagtctccatcctccctgtctgcatctgtaggagacagagtcaccatcacttgccgggcaagtcagggcattagaaatgatttaggctggtttcagcagaaaccaggaaaagcccctaagcgcctgatctatgttgcatccaatttacaaagtggggtcccatcaaggttcagcggcagtggatctgggacagaattcactctcacaatcagcagcctgcagcctgaagattttgcaacttattactgtctacagcatagtaattaccctccgtggacgttcggccaagggaccaaggtggaaatcaaa

SEQ ID NO: 314 - protein, 108 aa, Artificial Sequence
  Synthetic
DIQMTQSPSSLSASVGDRVTITCRASQGIRNDLGWFQQKPGKAPKRLIYVASNLQSGVPSRFSGSGSGTEFTLTISSLQPEDFATYYCLQHSNYPPWTFGQGTKVEIK

SEQ ID NO: 315 - DNA, 18 bp, Artificial Sequence
  Synthetic
cagggcattagaaatgat

SEQ ID NO: 316 - protein, 6 aa, Artificial Sequence
  Synthetic
QGIRND

SEQ ID NO: 317 - DNA, 9 bp, Artificial Sequence
  Synthetic
gttgcatcc

SEQ ID NO: 318 - protein, 3 aa, Artificial Sequence
  Synthetic
VAS

SEQ ID NO: 319 - DNA, 30 bp, Artificial Sequence
  Synthetic
ctacagcatagtaattaccctccgtggacg

SEQ ID NO: 320 - protein, 10 aa, Artificial Sequence
  Synthetic
LQHSNYPPWT

SEQ ID NO: 321 - DNA, 354 bp, Artificial Sequence
  Synthetic
caggtgcagctggtggagtctgggggaggcgtggtccagcctgggaggtccctgagactctcctgtgcagcgtctggattcattttcagtagctatggcatgcactgggtccgccaggctccaggcaaggggctggagtgggtggcagttatatggaatgatggaagtaataaatactctgcagactccgtgaagggccgattcaccgtctccagggacaattccaagaacaccctgtatctgcaaatgaacagtctgaaagccgaggacacggctgtgtattactgtgcgagagacggggagtgggaggtttttgactactggggccagggaaccctggtcaccgtctcctca

SEQ ID NO: 322 - protein, 118 aa, Artificial Sequence
  Synthetic
QVQLVESGGGVVQPGRSLRLSCAASGFIFSSYGMHWVRQAPGKGLEWVAVIWNDGSNKYSADSVKGRFTVSRDNSKNTLYLQMNSLKAEDTAVYYCARDGEWEVFDYWGQGTLVTVSS

SEQ ID NO: 323 - DNA, 24 bp, Artificial Sequence
  Synthetic
ggattcattttcagtagctatggc

SEQ ID NO: 324 - protein, 8 aa, Artificial Sequence
  Synthetic
GFIFSSYG

SEQ ID NO: 325 - DNA, 24 bp, Artificial Sequence
  Synthetic
atatggaatgatggaagtaataaa

SEQ ID NO: 326 - protein, 8 aa, Artificial Sequence
  Synthetic
IWNDGSNK

SEQ ID NO: 327 - DNA, 33 bp, Artificial Sequence
  Synthetic
gcgagagacggggagtgggaggtttttgactac

SEQ ID NO: 328 - protein, 11 aa, Artificial Sequence
  Synthetic
ARDGEWEVFDY

SEQ ID NO: 329 - DNA, 324 bp, Artificial Sequence
  Synthetic
gacatagtgatgacgcagtctccagtcaccctgtctgcgtctccaggggaaagagccaccctctcctgcagggccagtcagagtgttcgaagcaacttagcctggtaccaggagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataatgactggcctccgtggacgttcggccaagggaccaaggtggaaatcaaa

SEQ ID NO: 330 - protein, 108 aa, Artificial Sequence
  Synthetic
DIVMTQSPVTLSASPGERATLSCRASQSVRSNLAWYQEKPGQAPRLLIYGASTRATGIPARFSGSGSGTEFTLTISSLQSEDFAVYYCQQYNDWPPWTFGQGTKVEIK

SEQ ID NO: 331 - DNA, 18 bp, Artificial Sequence
  Synthetic
cagagtgttcgaagcaac

SEQ ID NO: 332 - protein, 6 aa, Artificial Sequence
  Synthetic
QSVRSN

SEQ ID NO: 333 - DNA, 9 bp, Artificial Sequence
  Synthetic
ggtgcatcc

SEQ ID NO: 334 - protein, 3 aa, Artificial Sequence
  Synthetic
GAS

SEQ ID NO: 335 - DNA, 30 bp, Artificial Sequence
  Synthetic
cagcagtataatgactggcctccgtggacg

SEQ ID NO: 336 - protein, 10 aa, Artificial Sequence
  Synthetic
QQYNDWPPWT

SEQ ID NO: 337 - DNA, 372 bp, Artificial Sequence
  Synthetic
caggtgcagctggtgcagtctggggctgaggtgaagaagcctggggcctcagtgaaggtctcctgcaaagtttccggatacaccctcactgaattatccatgcactgggtgcgacaggctcctggaaaagggcttgagtggatgggaggttttgatcctgaagatggtgaaacaatctacgcacagaagttccagggcagactcaccatgaccgaggacacatctacagacacagcctacatggagctgagcagcctgagatctgaggacacggccgtgtattactgtgcaacagacgattacgatattttgacttcttatccttacaatatggacgtctggggccaagggaccacggtcaccgtctcctca

SEQ ID NO: 338 - protein, 124 aa, Artificial Sequence
  Synthetic
QVQLVQSGAEVKKPGASVKVSCKVSGYTLTELSMHWVRQAPGKGLEWMGGFDPEDGETIYAQKFQGRLTMTEDTSTDTAYMELSSLRSEDTAVYYCATDDYDILTSYPYNMDVWGQGTTVTVSS

SEQ ID NO: 339 - DNA, 24 bp, Artificial Sequence
  Synthetic
ggatacaccctcactgaattatcc

SEQ ID NO: 340 - protein, 8 aa, Artificial Sequence
  Synthetic
GYTLTELS

SEQ ID NO: 341 - DNA, 24 bp, Artificial Sequence
  Synthetic
tttgatcctgaagatggtgaaaca

SEQ ID NO: 342 - protein, 8 aa, Artificial Sequence
  Synthetic
FDPEDGET

SEQ ID NO: 343 - DNA, 51 bp, Artificial Sequence
  Synthetic
gcaacagacgattacgatattttgacttcttatccttacaatatggacgtc

SEQ ID NO: 344 - protein, 17 aa, Artificial Sequence
  Synthetic
ATDDYDILTSYPYNMDV

SEQ ID NO: 345 - DNA, 321 bp, Artificial Sequence
  Synthetic
gacatccagatgacccagtctccatcctccctgtctgcatctgtaggagacagagtcaccatcacttgccgggcaagtcagagcattaacaactatttaaattggtatcagcagaaaccagggaaagcccctaagctcctgatctatgctgcatccagtttgcaaagtggggtcccatcaaggttcagtggcagtggatctgggacagatttcactctcaccatcagcagccttcaacctgaagattttggaacttactactgtcaacagagtgacagtaccccattcactttcggccctgggaccaaagtggatatcaaa

SEQ ID NO: 346 - protein, 107 aa, Artificial Sequence
  Synthetic
DIQMTQSPSSLSASVGDRVTITCRASQSINNYLNWYQQKPGKAPKLLIYAASSLQSGVPSRFSGSGSGTDFTLTISSLQPEDFGTYYCQQSDSTPFTFGPGTKVDIK

SEQ ID NO: 347 - DNA, 18 bp, Artificial Sequence
  Synthetic
cagagcattaacaactat

SEQ ID NO: 348 - protein, 6 aa, Artificial Sequence
  Synthetic
QSINNY

SEQ ID NO: 349 - DNA, 9 bp, Artificial Sequence
  Synthetic
gctgcatcc

SEQ ID NO: 350 - protein, 3 aa, Artificial Sequence
  Synthetic
AAS

SEQ ID NO: 351 - DNA, 27 bp, Artificial Sequence
  Synthetic
caacagagtgacagtaccccattcact

SEQ ID NO: 352 - protein, 9 aa, Artificial Sequence
  Synthetic
QQSDSTPFT

SEQ ID NO: 353 - DNA, 363 bp, Artificial Sequence
  Synthetic
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaattattactatgcagcctccgtgaagggccgattcaccatctccagagacaattccgagaacacgctgtatctgcaaatgaacagactgagagccgaggacacggctgtgtattactgtgcgagagaggggactggaagtacggaggactactttgactactggggccagggaaccctggtcaccgtctcctca

SEQ ID NO: 354 - protein, 121 aa, Artificial Sequence
  Synthetic
QVQLVESGGGVVQPGRSLRLSCAASGFTFSSYGMHWVRQAPGKGLEWVAVIWYDGSNYYYAASVKGRFTISRDNSENTLYLQMNRLRAEDTAVYYCAREGTGSTEDYFDYWGQGTLVTVSS

SEQ ID NO: 355 - DNA, 24 bp, Artificial Sequence
  Synthetic
ggattcaccttcagtagctatggc

SEQ ID NO: 356 - protein, 8 aa, Artificial Sequence
  Synthetic
GFTFSSYG

SEQ ID NO: 357 - DNA, 24 bp, Artificial Sequence
  Synthetic
atatggtatgatggaagtaattat

SEQ ID NO: 358 - protein, 8 aa, Artificial Sequence
  Synthetic
IWYDGSNY

SEQ ID NO: 359 - DNA, 42 bp, Artificial Sequence
  Synthetic
gcgagagaggggactggaagtacggaggactactttgactac

SEQ ID NO: 360 - protein, 14 aa, Artificial Sequence
  Synthetic
AREGTGSTEDYFDY

SEQ ID NO: 361 - DNA, 324 bp, Artificial Sequence
  Synthetic
gaaattgtgttgacacagtctccagccaccctgtctttgtctccaggggaaagagccaccctctcctgcagggccagtcagagtgttagcagcttcttagcctggtatcaacagaaacctggccaggctcccaggctcctcatctatgatgcatccaacagggccactggcatcccagtcaggttcagtggcagtgggtctgggacagacttcactctcaccatcagcagcctagagcctgaagattttgcagtttattactgtcagcagcgtagcaactggcctccgtacacttttggccaggggaccaagctggagatcaaa

SEQ ID NO: 362 - protein, 108 aa, Artificial Sequence
  Synthetic
EIVLTQSPATLSLSPGERATLSCRASQSVSSFLAWYQQKPGQAPRLLIYDASNRATGIPVRFSGSGSGTDFTLTISSLEPEDFAVYYCQQRSNWPPYTFGQGTKLEIK

SEQ ID NO: 363 - DNA, 18 bp, Artificial Sequence
  Synthetic
cagagtgttagcagcttc

SEQ ID NO: 364 - protein, 6 aa, Artificial Sequence
  Synthetic
QSVSSF

SEQ ID NO: 365 - DNA, 9 bp, Artificial Sequence
  Synthetic
gatgcatcc

SEQ ID NO: 366 - protein, 3 aa, Artificial Sequence
  Synthetic
DAS

SEQ ID NO: 367 - DNA, 30 bp, Artificial Sequence; identical to SEQ ID NO: 15
  Synthetic

SEQ ID NO: 368 - protein, 10 aa, Artificial Sequence
  Synthetic
QQRSNWPPYT

SEQ ID NO: 369 - DNA, 372 bp, Artificial Sequence
  Synthetic
caggtccagctggtacagtctggggctgaggtgaagaagcctggggcctcagtgaaggtctcctgcaaggtttccggatacaccctcactgaattatccatgcactgggtgcgacaggctcctggaaaagggcttgagtggatgggaggttttgatcctgaagatggggaaacaatctacgcacagaagttccagggcagagtcaccatgaccgaggacacatctacagaaacagcctacatggagctgagcagcctgagatctgaggacacggccgtgtattactgtgcaacagacgattacgatattttgactgattatccttacaatatggacgtctggggccaagggaccacggtcaccgtctcctca

SEQ ID NO: 370 - protein, 124 aa, Artificial Sequence
  Synthetic
QVQLVQSGAEVKKPGASVKVSCKVSGYTLTELSMHWVRQAPGKGLEWMGGFDPEDGETIYAQKFQGRVTMTEDTSTETAYMELSSLRSEDTAVYYCATDDYDILTDYPYNMDVWGQGTTVTVSS

SEQ ID NO: 371 - DNA, 24 bp, Artificial Sequence
  Synthetic
ggatacaccctcactgaattatcc

SEQ ID NO: 372 - protein, 8 aa, Artificial Sequence
  Synthetic
GYTLTELS

SEQ ID NO: 373 - DNA, 24 bp, Artificial Sequence
  Synthetic
tttgatcctgaagatggggaaaca

SEQ ID NO: 374 - protein, 8 aa, Artificial Sequence
  Synthetic
FDPEDGET

SEQ ID NO: 375 - DNA, 51 bp, Artificial Sequence
  Synthetic
gcaacagacgattacgatattttgactgattatccttacaatatggacgtc

SEQ ID NO: 376 - protein, 17 aa, Artificial Sequence
  Synthetic
ATDDYDILTDYPYNMDV

SEQ ID NO: 377 - DNA, 321 bp, Artificial Sequence
  Synthetic
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagatcttgcaacttactactgtcaacagagttccagtaccccattcactttcggccctgggaccaaagtggatatcaaa

SEQ ID NO: 378 - protein, 107 aa, Artificial Sequence
  Synthetic
DIQMTQSPSSLSASVGDRVTITCRASQSISSYLNWYQQKPGKAPKLLIYAASSLQSGVPSRFSGSGSGTDFTLTISSLQPEDLATYYCQQSSSTPFTFGPGTKVDIK

SEQ ID NO: 379 - DNA, 18 bp, Artificial Sequence
  Synthetic
cagagcattagcagctat

SEQ ID NO: 380 - protein, 6 aa, Artificial Sequence
  Synthetic
QSISSY

SEQ ID NO: 381 - DNA, 9 bp, Artificial Sequence
  Synthetic
gctgcatcc

SEQ ID NO: 382 - protein, 3 aa, Artificial Sequence
  Synthetic
AAS

SEQ ID NO: 383 - DNA, 27 bp, Artificial Sequence
  Synthetic
caacagagttccagtaccccattcact

SEQ ID NO: 384 - protein, 9 aa, Artificial Sequence
  Synthetic
QQSSSTPFT

SEQ ID NO: 385 - DNA, 357 bp, Artificial Sequence
  Synthetic
caggtgcagctggtggagtcggggggaggcttggtcaaacctggagggtccctgagactctcctgtgcagcctctggattcaccttcagtgactactacatgagctggatccgccaggctccagggaaggggctggagtgggtttcatacattggtactcgtggtagttccatatactacgcagactctttgaagggccgattcaccatctccagggacaacgccaagaactcactatatctgcaaatgaacagcctgagagccgaggacacggccgtgtattactgtgcgagagacccaactggaacctcctactttgactactggggccagggtaccctggtcaccgtctcctca

SEQ ID NO: 386 - protein, 119 aa, Artificial Sequence
  Synthetic
QVQLVESGGGLVKPGGSLRLSCAASGFTFSDYYMSWIRQAPGKGLEWVSYIGTRGSSIYYADSLKGRFTISRDNAKNSLYLQMNSLRAEDTAVYYCARDPTGTSYFDYWGQGTLVTVSS

SEQ ID NO: 387 - DNA, 24 bp, Artificial Sequence
  Synthetic
ggattcaccttcagtgactactac

SEQ ID NO: 388 - protein, 8 aa, Artificial Sequence
  Synthetic
GFTFSDYY

SEQ ID NO: 389 - DNA, 24 bp, Artificial Sequence
  Synthetic
attggtactcgtggtagttccata

SEQ ID NO: 390 - protein, 8 aa, Artificial Sequence
  Synthetic
IGTRGSSI

SEQ ID NO: 391 - DNA, 36 bp, Artificial Sequence
  Synthetic
gcgagagacccaactggaacctcctactttgactac

SEQ ID NO: 392 - protein, 12 aa, Artificial Sequence
  Synthetic
ARDPTGTSYFDY

SEQ ID NO: 393 - DNA, 324 bp, Artificial Sequence
  Synthetic
gaaatagtgatgacacagtctccagccaccctgtctgtgtctccaggggaaagagccaccctctcctgcagggccagtcagagtcttagcagcaacttagcctggtaccagcagaaacctggccaggctcccaggctcctcatctatggtgcatccaccagggccactggtatcccagccaggttcagtggcagtgggtctgggacagagttcactctcaccatcaacagcctgcagtctgaagactttgcagtttattactgtcagcagtataataactggcctccgtacacttttggccaggggaccaagctggagatcaaa

SEQ ID NO: 394 - protein, 108 aa, Artificial Sequence
  Synthetic
EIVMTQSPATLSVSPGERATLSCRASQSLSSNLAWYQQKPGQAPRLLIYGASTRATGIPARFSGSGSGTEFTLTINSLQSEDFAVYYCQQYNNWPPYTFGQGTKLEIK

SEQ ID NO: 395 - DNA, 18 bp, Artificial Sequence
  Synthetic
cagagtcttagcagcaac

SEQ ID NO: 396 - protein, 6 aa, Artificial Sequence
  Synthetic
QSLSSN

SEQ ID NO: 397 - DNA, 9 bp, Artificial Sequence
  Synthetic
ggtgcatcc

SEQ ID NO: 398 - protein, 3 aa, Artificial Sequence
  Synthetic
GAS

SEQ ID NO: 399 - DNA, 30 bp, Artificial Sequence; identical to SEQ ID NO: 95
  Synthetic

SEQ ID NO: 400 - protein, 10 aa, Artificial Sequence
  Synthetic
QQYNNWPPYT

SEQ ID NO: 401 - DNA, 357 bp, Artificial Sequence
  Synthetic
caggtgcagctggtggagtctgggggaggcgtggtccagcctgggaggtccctgagactctcctgtgcagcgtctggattcaccttcagtagttatggaatgcactgggtccgccaggctccaggcaaggggctggagtgggtggcagttatatggtatgatggaagtaataaatactatgcagactccgtgaagggccgattcatcatctccagagacaattccaagaacatgatgtatctgcaaatgaacagcctgagagccgaggacacggctgtgtattactgtgcgagagaagataactggaactacgcctttgactactggggccagggaaccctggtcaccgtctcctca

SEQ ID NO: 402 - protein, 119 aa, Artificial Sequence
  Synthetic
QVQLVESGGGVVQPGRSLRLSCAASGFTFSSYGMHWVRQAPGKGLEWVAVIWYDGSNKYYADSVKGRFIISRDNSKNMMYLQMNSLRAEDTAVYYCAREDNWNYAFDYWGQGTLVTVSS

SEQ ID NO: 403 - DNA, 24 bp, Artificial Sequence
  Synthetic
ggattcaccttcagtagttatgga

SEQ ID NO: 404 - protein, 8 aa, Artificial Sequence
  Synthetic
GFTFSSYG

SEQ ID NO: 405 - DNA, 24 bp, Artificial Sequence
  Synthetic
atatggtatgatggaagtaataaa

SEQ ID NO: 406 - protein, 8 aa, Artificial Sequence
  Synthetic
IWYDGSNK

SEQ ID NO: 407 - DNA, 36 bp, Artificial Sequence
  Synthetic
gcgagagaagataactggaactacgcctttgactac

SEQ ID NO: 408 - protein, 12 aa, Artificial Sequence
  Synthetic
AREDNWNYAFDY

SEQ ID NO: 409 - DNA, 324 bp, Artificial Sequence
  Synthetic
gaaatagtgatgacgcagtctccagccaccctgtctgtgtctccaggggaaagagccaccctctcctgcagggccagtcagagtattagcagcaacttagcctggtaccagcagaaacctggccaggctcccaggctcctcatctatgattcatccaccagggccactggaatcccagccaggttcagtggcagtgggtctggaacacaattcactctcaccatcagcagcctgcagtctgaagattttgcactttattactgtcagcagtatagtaactggcctccattcactttcggccctgggaccaaagtggatatcaaa

SEQ ID NO: 410 - protein, 108 aa, Artificial Sequence
  Synthetic
EIVMTQSPATLSVSPGERATLSCRASQSISSNLAWYQQKPGQAPRLLIYDSSTRATGIPARFSGSGSGTQFTLTISSLQSEDFALYYCQQYSNWPPFTFGPGTKVDIK

SEQ ID NO: 411 - DNA, 18 bp, Artificial Sequence
  Synthetic
cagagtattagcagcaac

SEQ ID NO: 412 - protein, 6 aa, Artificial Sequence
  Synthetic
QSISSN

SEQ ID NO: 413 - DNA, 9 bp, Artificial Sequence
  Synthetic
gattcatcc

SEQ ID NO: 414 - protein, 3 aa, Artificial Sequence
  Synthetic
DSS

SEQ ID NO: 415 - DNA, 30 bp, Artificial Sequence
  Synthetic
cagcagtatagtaactggcctccattcact

SEQ ID NO: 416 - protein, 10 aa, Artificial Sequence
  Synthetic
QQYSNWPPFT

SEQ ID NO: 417 - DNA, 372 bp, Artificial Sequence
  Synthetic
caggtccagctggtacagtctggggctgaggtgaagaagcctggggcctcagtgaaggtctcctgcaaggtttccggatacaccctcactgaattatccatgcactgggtgcgacaggctcctggaaaagggcttgagtggatgggaggttttgatcctgaagatggtgagacaatctacgcacagaagttccaggacagagtcaccatgaccgaggacacatctacagacacagcctacatggagctgagcagcctgagatctgaggacacggccgtgtattactgtgtaacagacgattacgaaattttgcctgaatatccttacaacatggacgtctggggccaagggaccacggtcaccgtctcctca

SEQ ID NO: 418 - protein, 124 aa, Artificial Sequence
  Synthetic
QVQLVQSGAEVKKPGASVKVSCKVSGYTLTELSMHWVRQAPGKGLEWMGGFDPEDGETIYAQKFQDRVTMTEDTSTDTAYMELSSLRSEDTAVYYCVTDDYEILPEYPYNMDVWGQGTTVTVSS

SEQ ID NO: 419 - DNA, 24 bp, Artificial Sequence
  Synthetic
ggatacaccctcactgaattatcc

SEQ ID NO: 420 - protein, 8 aa, Artificial Sequence
  Synthetic
GYTLTELS

SEQ ID NO: 421 - DNA, 24 bp, Artificial Sequence
  Synthetic
tttgatcctgaagatggtgagaca

SEQ ID NO: 422 - protein, 8 aa, Artificial Sequence
  Synthetic
FDPEDGET

SEQ ID NO: 423 - DNA, 51 bp, Artificial Sequence
  Synthetic
gtaacagacgattacgaaattttgcctgaatatccttacaacatggacgtc

SEQ ID NO: 424 - protein, 17 aa, Artificial Sequence
  Synthetic
VTDDYEILPEYPYNMDV

SEQ ID NO: 425 - DNA, 321 bp, Artificial Sequence
  Synthetic
gacatccagatgacccagtctccatcctccctgtctgcatctgtaggagacagagtcaccatcacttgccgggcaagtcagagcattagtacctatttaaattggtatcagcagaaaccagggaaagcccctaagctcctgatctatgctgcatccagtttgcaaagtggggtcccatcaaggttcagtggcagtggatctgggacagatttcactctcaccatccgcagtctgcaacctgaagattttgcaacttactactgtcaacagagtcacattaccccattcactttcggccctgggaccaaagtggatatcaaa

SEQ ID NO: 426 - protein, 107 aa, Artificial Sequence
  Synthetic
DIQMTQSPSSLSASVGDRVTITCRASQSISTYLNWYQQKPGKAPKLLIYAASSLQSGVPSRFSGSGSGTDFTLTIRSLQPEDFATYYCQQSHITPFTFGPGTKVDIK

SEQ ID NO: 427 - DNA, 18 bp, Artificial Sequence
  Synthetic
cagagcattagtacctat

SEQ ID NO: 428 - protein, 6 aa, Artificial Sequence
  Synthetic
QSISTY

SEQ ID NO: 429 - DNA, 9 bp, Artificial Sequence
  Synthetic
gctgcatcc

SEQ ID NO: 430 - protein, 3 aa, Artificial Sequence
  Synthetic
AAS

SEQ ID NO: 431 - DNA, 27 bp, Artificial Sequence
  Synthetic
caacagagtcacattaccccattcact

SEQ ID NO: 432 - protein, 9 aa, Artificial Sequence
  Synthetic
QQSHITPFT

SEQ ID NO: 433 - DNA, 351 bp, Artificial Sequence
  Synthetic
gaggtgcagctggtggagtctgggggaggcttggtccagcctggggggtccctgagactctcctgtgcagcctctggattcacgtttaatagcttttggatgagctgggtccgccaggctccagggaaggggctggagtgggtggccaacataaagcaggatggaagtgagaaatactatgtggactctgtgaagggccgattcaccatctccagagacaacgccaagaactcagtgtatctgcaaatgaacagcctgagagccgaggacacggctgtttattactgtgcgagagatctggtaacttcttttgactattggggccagggaaccctggtcaccgtctcctca

SEQ ID NO: 434 - protein, 117 aa, Artificial Sequence
  Synthetic
EVQLVESGGGLVQPGGSLRLSCAASGFTFNSFWMSWVRQAPGKGLEWVANIKQDGSEKYYVDSVKGRFTISRDNAKNSVYLQMNSLRAEDTAVYYCARDLVTSFDYWGQGTLVTVSS

SEQ ID NO: 435 - DNA, 24 bp, Artificial Sequence
  Synthetic
ggattcacgtttaatagcttttgg

SEQ ID NO: 436 - protein, 8 aa, Artificial Sequence
  Synthetic
GFTFNSFW

SEQ ID NO: 437 - DNA, 24 bp, Artificial Sequence
  Synthetic
ataaagcaggatggaagtgagaaa

SEQ ID NO: 438 - protein, 8 aa, Artificial Sequence
  Synthetic
IKQDGSEK

SEQ ID NO: 439 - DNA, 30 bp, Artificial Sequence
  Synthetic
gcgagagatctggtaacttcttttgactat

SEQ ID NO: 440 - protein, 10 aa, Artificial Sequence
  Synthetic
ARDLVTSFDY

SEQ ID NO: 441 - DNA, 321 bp, Artificial Sequence
  Synthetic
gacatccagatgacccagtctccatcctccctgtctgcatctgtaggagacagagtcaccatcacttgccaggcgagtcaggacattaacaactatttaaattggtatcagcagaaaccagggaaagcccctaagctcctgatctacgatgcatcctatttggaaacaggggtcccatcagggttcagtggaagtggatctgggacagattttactttcaccatcagcagcctgcagcctgaggatattgcaacatattactgtcaacagtatgatagtctcccgtacacttttggccaggggaccaagctggagatcaaa

SEQ ID NO: 442 - protein, 107 aa, Artificial Sequence
  Synthetic
DIQMTQSPSSLSASVGDRVTITCQASQDINNYLNWYQQKPGKAPKLLIYDASYLETGVPSGFSGSGSGTDFTFTISSLQPEDIATYYCQQYDSLPYTFGQGTKLEIK

SEQ ID NO: 443 - DNA, 18 bp, Artificial Sequence
  Synthetic
caggacattaacaactat

SEQ ID NO: 444 - protein, 6 aa, Artificial Sequence
  Synthetic
QDINNY

SEQ ID NO: 445 - DNA, 9 bp, Artificial Sequence
  Synthetic
gatgcatcc

SEQ ID NO: 446 - protein, 3 aa, Artificial Sequence
  Synthetic
DAS

SEQ ID NO: 447 - DNA, 27 bp, Artificial Sequence
  Synthetic
caacagtatgatagtctcccgtacact

SEQ ID NO: 448 - protein, 9 aa, Artificial Sequence
  Synthetic
QQYDSLPYT

SEQ ID NO: 449 - DNA, 363 bp, Artificial Sequence
  Synthetic
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcgattatatggtatgatggaagtaatagatactatgcagactccgtgaagggccggttcaccatctccagagacaattccaggaacacgctgtatctgcaaatgaacagcctgagagccgaggacacggctgtgtattactgtgcgcgagacgagtacggtgactacgactacccttttgactactggggccagggaaccctggtcaccgtctcctca

SEQ ID NO: 450 - protein, 121 aa, Artificial Sequence
  Synthetic
QVQLVESGGGVVQPGRSLRLSCAASGFTFSSYGMHWVRQAPGKGLEWVAIIWYDGSNRYYADSVKGRFTISRDNSRNTLYLQMNSLRAEDTAVYYCARDEYGDYDYPFDYWGQGTLVTVSS

SEQ ID NO: 451 - DNA, 24 bp, Artificial Sequence
  Synthetic
ggattcaccttcagtagctatggc

SEQ ID NO: 452 - protein, 8 aa, Artificial Sequence
  Synthetic
GFTFSSYG

SEQ ID NO: 453 - DNA, 24 bp, Artificial Sequence
  Synthetic
atatggtatgatggaagtaataga

SEQ ID NO: 454 - protein, 8 aa, Artificial Sequence
  Synthetic
IWYDGSNR

SEQ ID NO: 455 - DNA, 42 bp, Artificial Sequence
  Synthetic
gcgcgagacgagtacggtgactacgactacccttttgactac

SEQ ID NO: 456 - protein, 14 aa, Artificial Sequence
  Synthetic
ARDEYGDYDYPFDY

SEQ ID NO: 457 - DNA, 324 bp, Artificial Sequence
  Synthetic
gacatccagatgacccagtctccatcctccctgtctgcatctgtaggagacagagtcaccatcacttgccgggcaagtcagggcattagaaatgatttaggctggtttcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatacttaccctccattcactttcggccctgggaccaaagtggatatcaaa

SEQ ID NO: 458 - protein, 108 aa, Artificial Sequence
  Synthetic
DIQMTQSPSSLSASVGDRVTITCRASQGIRNDLGWFQQKPGKAPKRLIYAASSLQSGVPSRFSGSGSGTEFTLTISSLQPEDFATYYCLQHNTYPPFTFGPGTKVDIK

SEQ ID NO: 459 - DNA, 18 bp, Artificial Sequence
  Synthetic
cagggcattagaaatgat

SEQ ID NO: 460 - protein, 6 aa, Artificial Sequence
  Synthetic
QGIRND

SEQ ID NO: 461 - DNA, 9 bp, Artificial Sequence
  Synthetic
gctgcatcc

SEQ ID NO: 462 - protein, 3 aa, Artificial Sequence
  Synthetic
AAS

SEQ ID NO: 463 - DNA, 30 bp, Artificial Sequence
  Synthetic
ctacagcataatacttaccctccattcact

SEQ ID NO: 464 - protein, 10 aa, Artificial Sequence
  Synthetic
LQHNTYPPFT

SEQ ID NO: 465 - DNA, 360 bp, Artificial Sequence
  Synthetic
caggtgcagctggtggagtctgggggaggcgtggtccagcctgggaggtccctgagactctcctgtgcagcgtctggattcaccttcagtaactatggcatgcactgggtccgccaggttccaggcaaggggctggagtgggtggcagttatatggtatgatggaagtaataaatactatgcagactccgtgaagggccgattcaccatctccagagacaattccaagaacacgctgtatctgcaaatgaacagtgtgagagccgaggacacggctgtatactactgtgcgagagatgactacggtgactccccggggtttgactactggggccagggaaccctggtcaccgtctcctca

SEQ ID NO: 466 - protein, 120 aa, Artificial Sequence
  Synthetic
QVQLVESGGGVVQPGRSLRLSCAASGFTFSNYGMHWVRQVPGKGLEWVAVIWYDGSNKYYADSVKGRFTISRDNSKNTLYLQMNSVRAEDTAVYYCARDDYGDSPGFDYWGQGTLVTVSS

SEQ ID NO: 467 - DNA, 24 bp, Artificial Sequence
  Synthetic
ggattcaccttcagtaactatggc

SEQ ID NO: 468 - protein, 8 aa, Artificial Sequence
  Synthetic
GFTFSNYG

SEQ ID NO: 469 - DNA, 24 bp, Artificial Sequence
  Synthetic
atatggtatgatggaagtaataaa

SEQ ID NO: 470 - protein, 8 aa, Artificial Sequence
  Synthetic
IWYDGSNK

SEQ ID NO: 471 - DNA, 39 bp, Artificial Sequence
  Synthetic
gcgagagatgactacggtgactccccggggtttgactac

SEQ ID NO: 472 - protein, 13 aa, Artificial Sequence
  Synthetic
ARDDYGDSPGFDY

SEQ ID NO: 473 - DNA, 324 bp, Artificial Sequence
  Synthetic
gaaatagtgatgacgcagtctccagccaccctgtctgtgtctccaggggaaagagccaccctctcctgcagggccagtcagagtgttagcagcagcttagcctggtaccagcagaaacctggccaggctcccaggctcctcatctatgatgcatccaccagggccactggtatcccagccaggttcagtggcagtgggtctgggacagagttcactctcaccatcagcggcctgcagtctgaagattttgcagtttattactgtcagcagtataataactggcctccgtggacgttcggccaagggaccaaggtggaaatcaaa

SEQ ID NO: 474 - protein, 108 aa, Artificial Sequence
  Synthetic
EIVMTQSPATLSVSPGERATLSCRASQSVSSSLAWYQQKPGQAPRLLIYDASTRATGIPARFSGSGSGTEFTLTISGLQSEDFAVYYCQQYNNWPPWTFGQGTKVEIK

SEQ ID NO: 475 - DNA, 18 bp, Artificial Sequence
  Synthetic
cagagtgttagcagcagc

SEQ ID NO: 476 - protein, 6 aa, Artificial Sequence
  Synthetic
QSVSSS

SEQ ID NO: 477 - DNA, 9 bp, Artificial Sequence
  Synthetic
gatgcatcc

SEQ ID NO: 478 - protein, 3 aa, Artificial Sequence
  Synthetic
DAS

SEQ ID NO: 479 - DNA, 30 bp, Artificial Sequence
  Synthetic
cagcagtataataactggcctccgtggacg

SEQ ID NO: 480 - protein, 10 aa, Artificial Sequence [sequence omitted]

SEQ ID NO: 481 - DNA, 363 bp, Artificial Sequence
  Synthetic
caggtgcagctggtggagtctgggggaggcgtggtccagcctgggaggtccctgagactctcctgtgcagcgtctggattcaccttcagtaactatggcatgcattgggtccgccaggctccaggcaaggggctggagtgggtggcagttatatggtatgatggaagtaataaatactatgcagactctgtgaagggccgattcaccatctccagagacaattccaggaaaacgctgtatctgcaaatgaacagcctgagagccgaggacacggctatttattactgtacgagagacgactacggtgactacgactacccttttgactactggggccagggaaccctggtcaccgtctcctca

SEQ ID NO: 482 - protein, 121 aa, Artificial Sequence
  Synthetic
QVQLVESGGGVVQPGRSLRLSCAASGFTFSNYGMHWVRQAPGKGLEWVAVIWYDGSNKYYADSVKGRFTISRDNSRKTLYLQMNSLRAEDTAIYYCTRDDYGDYDYPFDYWGQGTLVTVSS

SEQ ID NO: 483 - DNA, 24 bp, Artificial Sequence
  Synthetic
ggattcaccttcagtaactatggc

SEQ ID NO: 484 - protein, 8 aa, Artificial Sequence
  Synthetic
GFTFSNYG

SEQ ID NO: 485 - DNA, 24 bp, Artificial Sequence
  Synthetic
atatggtatgatggaagtaataaa

SEQ ID NO: 486 - protein, 8 aa, Artificial Sequence
  Synthetic
IWYDGSNK

SEQ ID NO: 487 - DNA, 42 bp, Artificial Sequence
  Synthetic
acgagagacgactacggtgactacgactacccttttgactac

SEQ ID NO: 488 - protein, 14 aa, Artificial Sequence
  Synthetic
TRDDYGDYDYPFDY

SEQ ID NO: 489 - DNA, 324 bp, Artificial Sequence
  Synthetic
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcactggatctgggacagaattcactctcacaatcagcagcctgcagcctgaagattttgcaacttattactgtctacaacataatagttaccctccattcactttcggccctgggaccaaagtggatatcaaa

SEQ ID NO: 490 - protein, 108 aa, Artificial Sequence
  Synthetic
DIQMTQSPSSLSASVGDRVTITCRASQGIRNDLGWYQQKPGKAPKRLIYAASSLQSGVPSRFSGTGSGTEFTLTISSLQPEDFATYYCLQHNSYPPFTFGPGTKVDIK

SEQ ID NO: 491 - DNA, 18 bp, Artificial Sequence
  Synthetic
cagggcattagaaatgat

SEQ ID NO: 492 - protein, 6 aa, Artificial Sequence
  Synthetic
QGIRND

SEQ ID NO: 493 - DNA, 9 bp, Artificial Sequence
  Synthetic
gctgcatcc

SEQ ID NO: 494 - protein, 3 aa, Artificial Sequence
  Synthetic
AAS

SEQ ID NO: 495 - DNA, 30 bp, Artificial Sequence
  Synthetic
ctacaacataatagttaccctccattcact

SEQ ID NO: 496 - protein, 10 aa, Artificial Sequence
  Synthetic
LQHNSYPPFT

SEQ ID NO: 497 - DNA, 360 bp, Artificial Sequence
  Synthetic
caggtgcagctggtggagtctgggggaggcgtggtccagcctgggaagtccctgagactctcctgtgcagcgtctggattcaccttcagtcgttatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgacgacacggctatgtattactgtgcgagagatgactacggtgactccccggggtttgacttctggggccagggaaccctggtcaccgtctcctca

SEQ ID NO: 498 - protein, 120 aa, Artificial Sequence
  Synthetic
QVQLVESGGGVVQPGKSLRLSCAASGFTFSRYGMHWVRQAPGKGLEWVAVIWYDGSNKYYADSVKGRFTISRDNSKNTLYLQMNSLRADDTAMYYCARDDYGDSPGFDFWGQGTLVTVSS

SEQ ID NO: 499 - DNA, 24 bp, Artificial Sequence
  Synthetic
ggattcaccttcagtcgttatggc

SEQ ID NO: 500 - protein, 8 aa, Artificial Sequence
  Synthetic
GFTFSRYG

SEQ ID NO: 501 - DNA, 24 bp, Artificial Sequence
  Synthetic
atatggtatgatggaagtaataaa

SEQ ID NO: 502 - protein, 8 aa, Artificial Sequence
  Synthetic
IWYDGSNK

SEQ ID NO: 503 - DNA, 39 bp, Artificial Sequence
  Synthetic
gcgagagatgactacggtgactccccggggtttgacttc

SEQ ID NO: 504 - protein, 13 aa, Artificial Sequence
  Synthetic
ARDDYGDSPGFDF

SEQ ID NO: 505 - DNA, 324 bp, Artificial Sequence
  Synthetic
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatgatgcatccaccagggccactggtatcccagccaggttcagtggcactgggtctgggacagagttcactctcaccatcagcagcctgcagtctgaagactttgcactttattactgtcagcagtatgatgactggcctccgtggacgttcggccaagggaccaaggtggaaatcaaa

SEQ ID NO: 506 - protein, 108 aa, Artificial Sequence
  Synthetic
EIVMTQSPATLSVSPGERATLSCRASQSVSSNLAWYQQKPGQAPRLLIYDASTRATGIPARFSGTGSGTEFTLTISSLQSEDFALYYCQQYDDWPPWTFGQGTKVEIK

SEQ ID NO: 507 - DNA, 18 bp, Artificial Sequence
  Synthetic
cagagtgttagcagcaac

SEQ ID NO: 508 - protein, 6 aa, Artificial Sequence
  Synthetic
QSVSSN

SEQ ID NO: 509 - DNA, 9 bp, Artificial Sequence
  Synthetic
gatgcatcc

SEQ ID NO: 510 - protein, 3 aa, Artificial Sequence
  Synthetic
DAS

SEQ ID NO: 511 - DNA, 30 bp, Artificial Sequence
  Synthetic
cagcagtatgatgactggcctccgtggacg

SEQ ID NO: 512 - protein, 10 aa, Artificial Sequence
  Synthetic
QQYDDWPPWT

SEQ ID NO: 513 - DNA, 363 bp, Artificial Sequence
  Synthetic
caggtgcagctggtggagtctgggggaggcgtggtccagcctgggaggtccctgagactctcctgtgcagcgtctggattcaccttcagtaactatggcatgcactgggtccgccaggctccaggcagggggctggagtgggtggcagttatatggtttgatggaagtaacaaatactatgcagactccgtgaagggccgattcaccatctccagagacaattccaagaacacgctgtatctgcaaatgaacagtctgagatccgaggacacggctgtgtattactgtacgagagacgactacggtgactacgactacccttttgactactggggccagggaaccctggtcaccgtctcctca

SEQ ID NO: 514 - protein, 121 aa, Artificial Sequence
  Synthetic
QVQLVESGGGVVQPGRSLRLSCAASGFTFSNYGMHWVRQAPGRGLEWVAVIWFDGSNKYYADSVKGRFTISRDNSKNTLYLQMNSLRSEDTAVYYCTRDDYGDYDYPFDYWGQGTLVTVSS

SEQ ID NO: 515 - DNA, 24 bp, Artificial Sequence
  Synthetic
ggattcaccttcagtaactatggc

SEQ ID NO: 516 - protein, 8 aa, Artificial Sequence
  Synthetic
GFTFSNYG

SEQ ID NO: 517 - DNA, 24 bp, Artificial Sequence
  Synthetic
atatggtttgatggaagtaacaaa

SEQ ID NO: 518 - protein, 8 aa, Artificial Sequence
  Synthetic
IWFDGSNK

SEQ ID NO: 519 - DNA, 42 bp, Artificial Sequence; identical to SEQ ID NO: 487
  Synthetic

SEQ ID NO: 520 - protein, 14 aa, Artificial Sequence
  Synthetic
TRDDYGDYDYPFDY

SEQ ID NO: 521 - DNA, 324 bp, Artificial Sequence
  Synthetic
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatccgggacagaattcactctcacaatcagcagcctgcagcctgaagattttgcaacttattactgtctacagtataatacttaccctccattcactttcggccctgggaccaaagtggatatcaaa

SEQ ID NO: 522 - protein, 108 aa, Artificial Sequence
  Synthetic
DIQMTQSPSSLSASVGDRVTITCRASQGIRNDLGWYQQKPGKAPKRLIYAASSLQSGVPSRFSGSGSGTEFTLTISSLQPEDFATYYCLQYNTYPPFTFGPGTKVDIK

SEQ ID NO: 523 - DNA, 18 bp, Artificial Sequence
  Synthetic
cagggcattagaaatgat

SEQ ID NO: 524 - protein, 6 aa, Artificial Sequence
  Synthetic
QGIRND

SEQ ID NO: 525 - DNA, 9 bp, Artificial Sequence
  Synthetic
gctgcatcc

SEQ ID NO: 526 - protein, 3 aa, Artificial Sequence
  Synthetic
AAS

SEQ ID NO: 527 - DNA, 30 bp, Artificial Sequence
  Synthetic
ctacagtataatacttaccctccattcact

SEQ ID NO: 528 - protein, 10 aa, Artificial Sequence
  Synthetic
LQYNTYPPFT

SEQ ID NO: 529 - DNA, 360 bp, Artificial Sequence
  Synthetic
caggtgcagctggtggagtctgggggaggcgtggtccagcctgggaggtccctgagactctcctgtgcagcgtctggattcaccttcagtgcctatggcatgcactgggtccgccagggtccaggcaaggggctggagtgggtggcagttatatggtatgatggaagtaataaatactatgcagactccgtgaagggccgattcaccatctccagagacaattccaagaacacgctgtatctgcaaatgaatagactgagagccgaggacacggctgtgtattactgtgcgagagatgactacggtgactccccggggtttgaccactggggccagggaaccctggtcactgtctcctca

SEQ ID NO: 530 - protein, 120 aa, Artificial Sequence
  Synthetic
QVQLVESGGGVVQPGRSLRLSCAASGFTFSAYGMHWVRQGPGKGLEWVAVIWYDGSNKYYADSVKGRFTISRDNSKNTLYLQMNRLRAEDTAVYYCARDDYGDSPGFDHWGQGTLVTVSS

SEQ ID NO: 531 - DNA, 24 bp, Artificial Sequence
  Synthetic
ggattcaccttcagtgcctatggc

SEQ ID NO: 532 - protein, 8 aa, Artificial Sequence
  Synthetic
GFTFSAYG

SEQ ID NO: 533 - DNA, 24 bp, Artificial Sequence
  Synthetic
atatggtatgatggaagtaataaa

SEQ ID NO: 534 - protein, 8 aa, Artificial Sequence
  Synthetic
IWYDGSNK

SEQ ID NO: 535 - DNA, 39 bp, Artificial Sequence
  Synthetic
gcgagagatgactacggtgactccccggggtttgaccac

SEQ ID NO: 536 - protein, 13 aa, Artificial Sequence
  Synthetic
ARDDYGDSPGFDH

SEQ ID NO: 537 - DNA, 324 bp, Artificial Sequence
  Synthetic
gaaatagtgatgacgcagtctccagccaccctgtctgtgtctccaggggaaagagccaccctctcctgcagggccagtcagagtgttagcagcgagttagcctggtatcagcagaaacctggccaggctcccaggctcctcttgtatggtgcatccaccagggccactggtatcccagccaggttcagtggcagtgggtctgggacagagttcactctcaccatcagcagcctgcagtctgaagattttgcagtttattactgtcagcagtatagtaactggcctccgtggacgttcggccaagggaccaaggtggaaatcaaa

SEQ ID NO: 538 - protein, 108 aa, Artificial Sequence
  Synthetic
EIVMTQSPATLSVSPGERATLSCRASQSVSSELAWYQQKPGQAPRLLLYGASTRATGIPARFSGSGSGTEFTLTISSLQSEDFAVYYCQQYSNWPPWTFGQGTKVEIK

SEQ ID NO: 539 - DNA, 18 bp, Artificial Sequence
  Synthetic
cagagtgttagcagcgag

SEQ ID NO: 540 - protein, 6 aa, Artificial Sequence
  Synthetic
QSVSSE

SEQ ID NO: 541 - DNA, 9 bp, Artificial Sequence
  Synthetic
ggtgcatcc

SEQ ID NO: 542 - protein, 3 aa, Artificial Sequence
  Synthetic
GAS

SEQ ID NO: 543 - DNA, 30 bp, Artificial Sequence
  Synthetic
cagcagtatagtaactggcctccgtggacg

SEQ ID NO: 544 - protein, 10 aa, Artificial Sequence
  Synthetic
QQYSNWPPWT

SEQ ID NO: 545 - DNA, 354 bp, Artificial Sequence
  Synthetic
caggtgcagctggtggagtctgggggaggcgtggtccagcctgggaggtccctgagactctcctgtgcagcgtctggattcaccttcagtacctatggcatgcactgggtccgccaggctccaggcaaggggctggagtgggtggcaattatatggtatgatggaagtaattactactatgcagactccgtgaagggccgattcaccatctccagagacaattccaagaacacgctgtatctgcaaatgaacagcctgagagccgaggacacggctgtgtattactgtgcgagagaggacaggaatgatgtttttgatatctggggccaagggacaatggtcaccgtctcttca

SEQ ID NO: 546 - protein, 118 aa, Artificial Sequence
  Synthetic
QVQLVESGGGVVQPGRSLRLSCAASGFTFSTYGMHWVRQAPGKGLEWVAIIWYDGSNYYYADSVKGRFTISRDNSKNTLYLQMNSLRAEDTAVYYCAREDRNDVFDIWGQGTMVTVSS

SEQ ID NO: 547 - DNA, 24 bp, Artificial Sequence
  Synthetic
ggattcaccttcagtacctatggc

SEQ ID NO: 548 - protein, 8 aa, Artificial Sequence
  Synthetic
GFTFSTYG

SEQ ID NO: 549 - DNA, 24 bp, Artificial Sequence
  Synthetic
atatggtatgatggaagtaattac

SEQ ID NO: 550 - protein, 8 aa, Artificial Sequence
  Synthetic
IWYDGSNY

SEQ ID NO: 551 - DNA, 33 bp, Artificial Sequence
  Synthetic
gcgagagaggacaggaatgatgtttttgatatc

SEQ ID NO: 552 - protein, 11 aa, Artificial Sequence
  Synthetic
AREDRNDVFDI

SEQ ID NO: 553 - DNA, 324 bp, Artificial Sequence
  Synthetic
gaaattgtgttgacacagtctccagccaccctgtctttgtctccaggggaaagagccaccctctcctgcagggccagtcagagtgttagcagcttcttagcctggtaccaacagaaacctggccaggctcccaggctcctcatctatgattcatccaacagggccactggcatcccagccaggttcagtggcagtgggtctgggacagacttcactctcaccatcagcagcctagagcctgaagattttgcagtttattactgtcagcagcttagcaactggcctccgatcaccttcggccaagggacacgactggagattaaa

SEQ ID NO: 554 - protein, 108 aa, Artificial Sequence
  Synthetic
EIVLTQSPATLSLSPGERATLSCRASQSVSSFLAWYQQKPGQAPRLLIYDSSNRATGIPARFSGSGSGTDFTLTISSLEPEDFAVYYCQQLSNWPPITFGQGTRLEIK

SEQ ID NO: 555 - DNA, 18 bp, Artificial Sequence
  Synthetic
cagagtgttagcagcttc

SEQ ID NO: 556 - protein, 6 aa, Artificial Sequence
  Synthetic
QSVSSF

SEQ ID NO: 557 - DNA, 9 bp, Artificial Sequence
  Synthetic
gattcatcc

SEQ ID NO: 558 - protein, 3 aa, Artificial Sequence
  Synthetic
DSS

SEQ ID NO: 559 - DNA, 30 bp, Artificial Sequence
  Synthetic
cagcagcttagcaactggcctccgatcacc

SEQ ID NO: 560 - protein, 10 aa, Artificial Sequence
  Synthetic
QQLSNWPPIT

SEQ ID NO: 561 - DNA, 348 bp, Artificial Sequence
  Synthetic
caggtgcagctgcaggagtcgggcccaggactggtgaagccttcacagaccctgtccctcacctgcactgtctctggtggctccatcagcagtggtggttactactggacctggatccgccagcacccagggaagggcctggagtggattgggtacatctattacagtgggagcacctactacaacccgtccctcaagagtcgagttaccatgtcagtagacacgtctaagaaccagttctccctgaagctgagctctgtgactgccgcggacacggccgtgtattactgtgcgagactactggattttgactactggggccagggaaccctggtcactgtctcctca

SEQ ID NO: 562 - protein, 116 aa, Artificial Sequence
  Synthetic
QVQLQESGPGLVKPSQTLSLTCTVSGGSISSGGYYWTWIRQHPGKGLEWIGYIYYSGSTYYNPSLKSRVTMSVDTSKNQFSLKLSSVTAADTAVYYCARLLDFDYWGQGTLVTVSS

SEQ ID NO: 563 - DNA, 30 bp, Artificial Sequence
  Synthetic
ggtggctccatcagcagtggtggttactac

SEQ ID NO: 564 - protein, 10 aa, Artificial Sequence
  Synthetic
GGSISSGGYY

SEQ ID NO: 565 - DNA, 21 bp, Artificial Sequence
  Synthetic
atctattacagtgggagcacc

SEQ ID NO: 566 - protein, 7 aa, Artificial Sequence
  Synthetic
IYYSGST

SEQ ID NO: 567 - DNA, 24 bp, Artificial Sequence
  Synthetic
gcgagactactggattttgactac

SEQ ID NO: 568 - protein, 8 aa, Artificial Sequence
  Synthetic
ARLLDFDY

SEQ ID NO: 569 - DNA, 321 bp, Artificial Sequence
  Synthetic
gccatccagatgacccagtctccatcctccctgtctgcatctgtaggagacagagtcactatcacttgccgggcaagtcagggcattagaaatgatttaggctggtatcagcagaaaccagggaaagcccctaagctcctgatctatgctgcatccagtttacaaagtggggtcccatcaaggttcagcggcagtggatctggcagagatttcactctcaccatcagcagcctgcagcctgaagattttgcctcttattactgtctacaagatcacaattacccgctcactttcggcggagggaccaaggtggagatcaaa

SEQ ID NO: 570 - protein, 107 aa, Artificial Sequence
  Synthetic
AIQMTQSPSSLSASVGDRVTITCRASQGIRNDLGWYQQKPGKAPKLLIYAASSLQSGVPSRFSGSGSGRDFTLTISSLQPEDFASYYCLQDHNYPLTFGGGTKVEIK

SEQ ID NO: 571 - DNA, 18 bp, Artificial Sequence
  Synthetic
cagggcattagaaatgat

SEQ ID NO: 572 - protein, 6 aa, Artificial Sequence
  Synthetic
QGIRND

SEQ ID NO: 573 - DNA, 9 bp, Artificial Sequence
  Synthetic
gctgcatcc

SEQ ID NO: 574 - protein, 3 aa, Artificial Sequence
  Synthetic
AAS

SEQ ID NO: 575 - DNA, 27 bp, Artificial Sequence
  Synthetic
ctacaagatcacaattacccgctcact

SEQ ID NO: 576 - protein, 9 aa, Artificial Sequence
  Synthetic
LQDHNYPLT

SEQ ID NO: 577 - DNA, 348 bp, Artificial Sequence
  Synthetic
caggtgcagctggtggagtctgggggagacgtggtccagcctgggaggtccctgagactctcctgtgcagcgtctggattcaccttcagtagctatggcatgcactgggtccgccaggctccaggcaaggggctggaatgggtggcaattatatggaatgatggaagtaataaatactatacagactccgtgaagggccgattcaccatctccagagacaattccaagaacactctatatctgcaaatgaatggcctgagagccgaggacacggctatatattactgtgcgcgagatcaggaccagtttgactactggggccagggaaccctggtcaccgtctcctca

SEQ ID NO: 578 - protein, 116 aa, Artificial Sequence
  Synthetic
QVQLVESGGDVVQPGRSLRLSCAASGFTFSSYGMHWVRQAPGKGLEWVAIIWNDGSNKYYTDSVKGRFTISRDNSKNTLYLQMNGLRAEDTAIYYCARDQDQFDYWGQGTLVTVSS

SEQ ID NO: 579 - DNA, 24 bp, Artificial Sequence
  Synthetic
ggattcaccttcagtagctatggc

SEQ ID NO: 580 - protein, 8 aa, Artificial Sequence
  Synthetic
GFTFSSYG

SEQ ID NO: 581 - DNA, 24 bp, Artificial Sequence
  Synthetic
atatggaatgatggaagtaataaa

SEQ ID NO: 582 - protein, 8 aa, Artificial Sequence
  Synthetic
IWNDGSNK

SEQ ID NO: 583 - DNA, 27 bp, Artificial Sequence
  Synthetic
gcgcgagatcaggaccagtttgactac

SEQ ID NO: 584 - protein, 9 aa, Artificial Sequence
  Synthetic
ARDQDQFDY

SEQ ID NO: 585 - DNA, 324 bp, Artificial Sequence
  Synthetic
gaaatagtgatgacgcagtctccagccaccctgtctgtgtctccaggggaaagagccaccctctcctgcagggccagtcagagtattagcacaaacttagcctggtaccggcagaaacctggccaggctccccggctcctcatctatgatgcatccaccagggccactggtatcccagccaggttcagtggcagtgggtctgggacagacttcactctcaccatcagcagcctgcagtctgaagattttgcagtttattactgtcagcagtatagtaactggcctccgtacacttttggccaggggaccaagctggagatcaaa

SEQ ID NO: 586 - protein, 108 aa, Artificial Sequence
  Synthetic
EIVMTQSPATLSVSPGERATLSCRASQSISTNLAWYRQKPGQAPRLLIYDASTRATGIPARFSGSGSGTDFTLTISSLQSEDFAVYYCQQYSNWPPYTFGQGTKLEIK

SEQ ID NO: 587 - DNA, 18 bp, Artificial Sequence
  Synthetic
cagagtattagcacaaac

SEQ ID NO: 588 - protein, 6 aa, Artificial Sequence
  Synthetic
QSISTN

SEQ ID NO: 589 - DNA, 9 bp, Artificial Sequence
  Synthetic
gatgcatcc

SEQ ID NO: 590 - protein, 3 aa, Artificial Sequence
  Synthetic
DAS

SEQ ID NO: 591 - DNA, 30 bp, Artificial Sequence
  Synthetic
cagcagtatagtaactggcctccgtacact

SEQ ID NO: 592 - protein, 10 aa, Artificial Sequence
  Synthetic
QQYSNWPPYT

SEQ ID NO: 593 - DNA, 555 bp, Homo sapiens
atgagccgcacagcctacacggtgggagccctgcttctcctcttggggaccctgctgccggctgctgaagggaaaaagaaagggtcccaaggtgccatccccccgccagacaaggcccagcacaatgactcagagcagactcagtcgccccagcagcctggctccaggaaccgggggcggggccaagggcggggcactgccatgcccggggaggaggtgctggagtccagccaagaggccctgcatgtgacggagcgcaaatacctgaagcgagactggtgcaaaacccagccgcttaagcagaccatccacgaggaaggctgcaacagtcgcaccatcatcaaccgcttctgttacggccagtgcaactctttctacatccccaggcacatccggaaggaggaaggttcctttcagtcctgctccttctgcaagcccaagaaattcactaccatgatggtcacactcaactgccctgaactacagccacctaccaagaagaagagagtcacacgtgtgaagcagtgtcgttgcatatccatcgatttggattaa

SEQ ID NO: 594 - protein, 184 aa, Homo sapiens
MSRTAYTVGALLLLLGTLLPAAEGKKKGSQGAIPPPDKAQHNDSEQTQSPQQPGSRNRGRGQGRGTAMPGEEVLESSQEALHVTERKYLKRDWCKTQPLKQTIHEEGCNSRTIINRFCYGQCNSFYIPRHIRKEEGSFQSCSFCKPKKFTTMMVTLNCPELQPPTKKKRVTRVKQCRCISIDLD

SEQ ID NO: 595 - protein, 184 aa, Artificial Sequence; identical to SEQ ID NO: 594
  Synthetic